(19) United States
(12) Patent Application Publication
(10) Pub. No.: US 2016/0333071 A1
(43) Pub. Date: Nov. 17, 2016

(54) Biological Pacemakers Incorporating HCN2 and SkM1 Genes

(71) Applicant: THE TRUSTEES OF COLUMBIA UNIVERSITY IN THE CITY OF NEW YORK, New York, NY (US)

(72) Inventors: Michael R. Rosen, New York, NY (US); Gerard J. Boink, Amsterdam (NL); Hanno L Tan, Amstelveen (NL); Richard B. Robinson, Cresskill, NJ (US); Ira S. Cohen, Stony Brook, NY (US); Peter R. Brink, Setauket, NY (US); Peter Danilo, Jr., Hopewell, NJ (US)

(73) Assignee: The Trustees of Columbia University in the City of New York, New York, NY (US)

(21) Appl. No.: 15/111,680

(22) PCT Filed: Jan. 14, 2015
(86) PCT No.: PCT/US15/11486

§ 371 Date: Jul. 14, 2016

Related U.S. Application Data
(60) Provisional application No. 61/927,018, filed on Jan. 14, 2014.

(51) Int. Cl. C07K 14/705; A61K 48/00; C12N 5/0775; A61K 38/17

(52) U.S. Cl. CPC C07K 14/705; A61K 38/177; A61K 48/0058; C12N 5/0662; A61K 48/00; C12N 2510/00

(57) ABSTRACT

It is demonstrated that hyperpolarization-activated cyclic nucleotide-gated (HCN)-based biological pacing, especially that achieved by transduction of the HCN2 gene into cardiac cells in vivo, was significantly improved by co-transduction of the skeletal muscle sodium channel 1 (SkMI) gene. Expression of both genes hyperpolarized the action potential (AP) threshold. When viral biological pacemaker constructs carrying genes for HCN2 and SkMI were injected into the heart of dogs in vivo, the pacemaker function was facilitated by the slow depolarizing HCN2 current and the hyperpolarized AP threshold generated by SkMI. This dual gene therapy provided both highly efficient pacing and a brisk autonomic response that is superior to those of previously developed gene- or cell-based approaches.

### CROSS-REFERENCE TO RELATED APPLICATIONS

The present application claims priority to U.S. Provisional Application No. 61/927,018, entitled “Biological Pacemakers Incorporating HCN2 and SkM1 Genes,” filed Jan. 14, 2014, the entire contents of which are hereby incorporated by reference as if fully set forth herein.

### STATEMENT OF GOVERNMENT INTEREST

This invention was made with government support under NHLBI HL-28958 and HL-094410. The government has certain rights in the invention.

### FIELD OF THE INVENTION

This invention is in the field of biological pacemakers.

### BACKGROUND OF THE INVENTION

In the United States, approximately 200,000 pacemakers are implanted annually. Five percent of these implantations result in serious complications requiring either a surgical revision or other invasive procedures. The cost to society that results from implanting electronic pacemakers is already estimated to be more than 2 billion USD annually, and expected to increase. Electronic pacemakers currently used clinically must be periodically monitored, maintained, and replaced. Leads fracture. Infection and interference can occur. Sites available for implantation often cannot optimize cardiac contraction. Units and leads available create anatomical problems when implanted in growing children. While there is rate responsiveness in some units, they do not adequately reflect the need for sympathetic and parasympathetic nerve input seen in patients during periods of altered exercise state or emotion.

The rhythm of the heart is initiated by the sinoatrial node (SAN), which is located in the right atrium of the heart. A particular combination of ion channels allows SAN cells to fire spontaneously and be under constant influence of the autonomic nervous system. Of particular importance in the generation of spontaneous activity within SAN cells is the pacemaker current I_{f}, which is encoded by hyperpolarization activated cyclic nucleotide gated (HCN) ion channels. From the sinus node, the electrical activation travels through the atrium and towards the atrioventricular node (AVN). This is a small structure (a few mm³) of specialized myocardial cells that passes electrical activity towards the ventricles with a delay. This delay is necessary to optimize hemodynamic performance of the heart; after atrial contraction the ventricles need time to fill before contracting. Both SAN and AVN are small and therefore prone to disabilities. Dysfunction of the SAN and/or AVN is currently treated with the implantation of an electronic pacemaker. Although these devices are sophisticated, they have disadvantages. They lack an adequate autonomic modulation of the heart rate, they have a limited battery life, their electrode position may be unstable or leads may fracture, and the device or the electrode may become infected. These limitations can be circumvented with biological pacemakers.

Biological pacemakers, inter alia, are expected to be better suitable for a child's heart, be more autonomic responsive, have optimal cardiac output, and provide a lifelong cure. The majority of the pacemaker patients (˜75%) now receive so called “dual camber” pacing—implanted in both the atria as well as the ventricle. Some patients receive single ventricular pacemakers. Biological pacemakers require no leads, no electrodes, and no battery pack; function should persist throughout life. Biological pacemakers can be implanted at individualized sites in patients to provide optimal cardiac output. There is no risk of lead fracture or of interference from extrinsic devices and implantation can be done at any age since growth of the subject's heart is not a problem.

### BRIEF DESCRIPTION OF THE DRAWINGS

Certain embodiments are directed to various methods for treating sinoatrial node dysfunction and/or atrioventricular conduction block in a subject, by administering to an area at, near or remote from the site of the sinoatrial node dysfunction and/or atrioventricular conduction block a composition comprising viral vector constructs or non-viral vector constructs comprising a gene encoding a biologically active hyperpolarization-activated cyclic nucleotide-gated channel (HCN1, HCN2 or HCN4 channel) or a biologically active fragment or variant thereof, and a gene encoding a biologically active voltage gated SkM1 sodium channel or a biologically active fragment or variant thereof, in a therapeutically effect amount and under conditions whereby the vector constructs transduce a plurality of cells located at or near the area and the encoded HCN2 and SkM1 channel genes are translated and expressed thereby treating the sinoatrial node dysfunction and/or atrioventricular conduction block. Other embodiments include administering an HCN channel gene that encodes a protein that has activation kinetics similar to HCN2 and cAMP responsiveness similar to HCN2, or a biologically active fragment or variant thereof, and a gene encoding a biologically active voltage gated sodium channel that has a depolarized inactivation relation similar to SkM1 or a biologically active fragment or variant thereof. In some embodiments the sodium gene encodes a neuronal isoform Nav 1.1, 1.2, 1.3, 1.6, 1.7 and 1.9 of the sodium channel gene. In some embodiments the composition comprises a viral vector is selected from the group consisting of an adenoviral vector, an adeno-associated viral (AAV) vector, or a retroviral vector. In other embodiments the lentiviral vector comprises a member selected from the group consisting of LV-CMV-X; LV-EF1α-X; LV-PGK; LV-cTnT-X; LV-cTnI; LV-CAG-X; LV-short troponin I-X; LV-CMV-immediate-early enhancer-ANF-X; LV-CMV-immediate-early enhancer-MLC2v; LV-αMHC-X; LV-MLC2v-X. The viral vector can be an adenoviral vector or a recombinant adeno-associated virus (rAAV) vector wherein each respective gene is operably linked to a promoter. Such vectors include AAV1, AAV2, AAV3, AAV4, AAV5, AAV6, AAV7, AAV8, AAV9, AAV10, AAV11, or AAV12 and a hybrid serotype thereof. In some embodiments the composition is administered to the subject intramyocardially or by infusion into the coronary vasculature. Still other embodiments include a population of cells selected from the group consisting of stem cells, cardiomyocytes, fibroblasts and skeletal muscle cells engineered to express a hyperpolarization-activated cyclic nucleotide-gated (HCN) channel or a biologically active fragment or variant thereof and a voltage gated sodium SkM1 channel or a biologically active fragment or variant thereof. In some embodiments the encoded HCN, HCN2, SkM1 or sodium channel gene is divided between two viral vectors.

FIG. 1A-I. HCN2/SkM1-Based Biological Pacemakers Exhibit Improved Function over HCN2 and SkM1. (A to C) Summary data for left bundle block (LBB)-injected animals receiving hyperpolarization-activated cyclic nucleotide-gated channel 2 (HCN2; n=12), skeletal muscle sodium channel 1 (SkM1; n=5 in A and B, n=6 in C), or HCN2/SkM1 (n=6). (A) Baseline beating rates on days 3 to 7 were faster in HCN2/SkM1-injected animals than in animals injected with only HCN2 or SkM1 (*). (B) Mean escape times on days 4 to 7 were shorter in HCN2/SkM1-injected animals than HCN2-injected animals (*). (C) Median percentage of electronically stimulated beats calculated over 24-h periods were significantly lower in HCN2/SkM1-injected animals than HCN2-injected animals. On days 4 to 7, electronic backup pacing was eliminated in HCN2/SkM1-injected animals. (D to F) Summary data for subepicardially injected animals receiving HCN2 (n=10), SkM1 (n=7), or HCN2/SkM1 (n=6). (D) Mean baseline beating rates. (E) Mean escape times. (F) Median percentage of electronically stimulated beats. (G to I) Summary data pooled for days 5 to 7. (G) Baseline beating rates in LBB-injected animals receiving HCN2/SkM1 were faster than in LBB-in-jected animals receiving either HCN2 or SkM1 (†). (H) Escape times in LBB-injected animals receiving SkM1 or HCN2/SkM1 were significantly shorter compared with those of the respective subepicardial injections (‡). Escape times of HCN2/SkM1-injected animals were significantly shorter than those of the respective HCN2 injections (*). (I) Median percentage of electronically stimulated beats was reduced to 0% in LBB-injected animals receiving HCN2/SkM1 and was significantly lower than in LBB-injected animals receiving HCN2 (*) or in subepicardially injected animals receiving HCN2/SkM1 (‡). Note that in panels C, F and I, error bars are presented as interquartile range. *†‡p<0.05.

FIG. 2A-E are graphs that illustrate HCN2/SkM1-based biological pacemakers injected into the LBB have faster maximal beating rates than those based on HCN2 or SkM1. (A) Maximal pace-mapped beating rates in LBB-injected animals. Maximal beating rates were faster in HCN2/SkM1 than HCN2 and SkM1 groups (+). (B) Summary data pooled for days 5 to 7. Maximal pace-mapped beating rates in LBB-injected animals were significantly faster in HCN2/SkM1 versus HCN2 or SkM1 (+). HCN2/SkM1. LBB-injected animals also had significantly faster maximal beating rates than respective subepicardially injected animals (‡; subepicardially injected animals: HCN2 n=10, SkM1 n=7, HCN2/SkM1 n=6; LBB-injected animals: HCN2 n=12, SkM1 n=6, HCN2/SkM1 n=6). (C to E) Left panels show beating rates for every beat during 8 min surrounding an episode of maximal pace-mapped beating rate recorded in LBB-injected animals. Right panels provide tracings of baseline and maximal beating rates of the recordings shown on the left. (C) Gradual warm up and cool down in an HCN2-injected animal. (D) Baseline bigeminy and stable maximal beating rate in an SkM1-injected animal. (E) Stable baseline beating and robust rate acceleration followed by cool down in an animal injected with HCN2/SkM1. Abbreviations as in FIG. 1. +‡p<0.05.

FIG. 3A-D. Detailed Analysis of Rhythms and Their Circadian Modulation. (A) Summary data on percentage of pace-mapped beats (percentage matching rhythm), nonmatching beats, bigeminal beats, and electronically paced beats in all ani-mals. Animals that showed <5% of matching beats or persistent bigeminy were excluded from subsequent analysis. (B to D; subepicardially injected animals: HCN2 n=9, SkM1 n=6, HCN2/SkM1 n=6; LBB-injected animals: HCN2 n=11, SkM1 n=4, HCN2/SkM1 n=6). (B) 24-h average rate of matching beats. (C) Summary data on morning/night modulation of pace-mapped beating rates. (D) Dependence on electronic backup pacing during morning/night. Note that percentage nonmatching, percentage bigeminy, and percentage paced are presented as median and interquartile range. Abbreviations as in FIG. 1. *p<0.05 versus respective HCN2. †p<0.05 versus respective HCN2 and SkM1. ‡p<0.05 versus respective myocardium. §p<0.05 versus respective HCN2 and HCN2/SkM1. !p<0.05 for morning versus night. (A) Summary data on percentage of pace-mapped beats (percentage matching rhythm), nonmatching beats, bigeminal beats, and electronically paced beats in all animals. Animals that showed <5% of matching beats or persistent bigeminy were excluded from subsequent analysis (B to D; subepicardially injected animals: HCN2 n=9, SkM1 n=6, HCN2/SkM1 n=6; LBB-injected animals: HCN2 n=11, SkM1 n=4, HCN2/SkM1 n=6). (B) 24-h average rate of matching beats. (C) Summary data on morning/night modulation of pace-mapped beating rates. (D) Dependence on electronic backup pacing during morning/night. Note that percentage nonmatching, percentage bigeminy, and percentage paced are presented as median and interquartile range. Abbreviations as in FIG. 1. *p<0.05 versus respective HCN2. †p<0.05 versus respective HCN2 and SkM1. ‡p<0.05 versus respective myocardium. §p<0.05 versus respective HCN2 and HCN2/SkM1. !p<0.05 for morning versus night.

FIG. 4A-C. Detailed Analysis of Heart Rate Variability and Response to Epinephrine Infusion. (A) Representative Poincaré plots of pace-mapped beats in 24-h Holter recordings of HCN2, SkM1, and HCN2/SkM1 LBB-injected animals. The middle panel (SkM1-injected animal) also defines SD of instantaneous RR-interval variability (SD1) and SD of long-term continuous RR-interval variability (SD2). (B) Summary data of SD1, SD2, and SD1:SD2. Animals that showed <5% of matching beats or persistent bigeminy were excluded from this analysis. Subepicardially injected animals: HCN2 n=9, SkM1 n=6, HCN2/SkM1 n=6; LBB-injected animals: HCN2 n=11, SkM1 n=4, HCN2/SkM1 n=6. (C) Summary data on the beating rates at baseline and during epinephrine infusion. Subepicardially injected animals: HCN2 n=10, SkM1 n=6, HCN2/SkM1 n=6; LBB-injected animals: HCN2 n=12, SkM1 n=4, HCN2/SkM1 n=6. Abbreviations as in FIG. 1. *p<0.05 versus respective HCN2. !p<0.05 for baseline versus epinephrine. †p<0.05 versus respective HCN2 and SkM1. ‡p<0.05 versus respective subepicardium.

FIG. 5A-B. Determining Beating Rates of HCN2/SkM1-Injected Preparations. (A) Representative microelectrode traces of LBB preparations from HCN2 (n=5), SkM1 (n=6), and HCN2/SkM1 (n=6) injected animals and noninjected zones (n=6) under control conditions, after graded application of isoproterenol and after isoproterenol (Iso) 0.1 J.M plus tetrodotoxin (TTX) 0.1 J.M. (B) Summary data for beating rates (left panel) and maximum diastolic potential (right panel). Abbreviations as in FIG. 1. *p<0.05 versus noninjected. †p<0.05 versus HCN2, SkM1, and noninjected. ‡p<0.05 for the combined groups with HCN2 (HCN2 and HCN2/SkM1) versus the combined groups without HCN2 (SkM1 and noninjected).

FIG. 6A-B. SkM1 Overexpression Shifts TP Negatively. Action potential (AP) parameters and threshold potential (TP) were registered from left ventricular subepicardial preparations isolated from HCN2-, SkM1-, or HCN2/SkM1-injected and noninjected regions. Preparations were paced at a cycle length of 1,000 ms with 2-ms current pulses at double threshold amplitude (51). A 30-ms test current pulse (S2) of variable amplitude was substituted for every 10th regular pulse. (A) Typical train of 9 APs initiated with 2-ms 2× threshold S1 current pulses followed by a 10th AP initiated by a 30-ms suprathreshold current pulse (S2). (B) Fast-sweep recordings of typical tracings of 30-ms subthreshold and suprathreshold current pulses in noninjected, HCN2-, SkM1-, and HCN2/SkM1-injected preparations.

In the present specification, the invention has been described with reference to specific embodiments thereof. It will, however, be evident that various modifications and changes may be made thereto without departing from the broader spirit and scope of the invention. The specification and drawings are, accordingly, to be regarded in an illustrative rather than a restrictive sense. The contents of all references, pending patent applications and published patents, cited throughout this application are hereby expressly incorporated by reference as if set forth herein in their entirety, except where terminology is not consistent with the definitions herein. Although specific terms are employed, they are used as in the art unless otherwise indicated.

### DEFINITIONS

All references cited herein, including the references cited therein, are incorporated by reference in their entirety as though fully set forth. Unless defined otherwise, technical and scientific terms used herein have the same meaning as commonly understood by one of ordinary skill in the art to which this invention belongs. The practice of the present invention will employ, unless indicated specifically to the contrary, conventional methods of molecular biology and recombinant DNA techniques within the skill of the art, many of which are described below for the purpose of illustration. Such techniques are fully explained in the literature. See, e.g., Singleton et al., Dictionary of Microbiology and Molecular Biology 3rd.sup.ed., J. Wiley & Sons (2001); March, Advanced Organic Chemistry Reactions, Mechanisms and Structure 5th.sup.ed., J. Wiley & Sons (2001); Sambrook & Russell, eds., Molecular Cloning: A Laboratory Manual 3rd ed., Cold Spring Harbor Laboratory Press (2001); Glover, ed., DNA Cloning: A Practical Approach, vol. I & II (2002); Gait, ed., Oligonucleotide Synthesis: A practical approach, Oxford University Press (1984); Herdewijn, ed., Oligonucleotide Synthesis: Methods and Applications, Humana Press (2005); Hames & Higgins, eds., Nucleic Acid Hybridisation: A Practical Approach, IRL Press (1985); Buzdin & Lukyanov, eds., Nucleic Acid Hybridization: Modern Applications, Springer (2007); Hames & Higgins, eds., Transcription and Translation: A Practical Approach, IRL Press (1984); Freshney, ed., Animal Cell Culture, Oxford UP (1986); Freshney, Culture of Animal Cells: A Manual of Basic Technique and Specialized Applications, 6th ed., John Wiley & Sons (2010); Perbal, A Practical Guide to Molecular Cloning, 3rd ed., Wiley-Liss (2014); Farrell, RNA Methodologies: A Laboratory Guide for Isolation and Characterization, 3rd ed., Elsevier/Focal Press (2005); Lilley & Dahlberg, eds., Methods in Enzymology: DNA Structures, Part A: Synthesis and Physical Analysis of DNA, Academic Press (1992); Harlow & Lane, Using Antibodies: A Laboratory Manual: Portable Protocol no. 1, Cold Spring Harbor Laboratory Press (1999); Harlow & Lane, eds., Antibodies: A Laboratory Manual, Cold Spring Harbor Laboratory Press (1988); Seethala & Fernandes, eds., Handbook of Drug Screening, Marcel Dekker (2001); and Roskams & Rodgers, eds., Lab Ref: A Handbook of Recipes, Reagents, and Other Reference Tools for Use at the Bench, Cold Spring Harbor Laboratory (2002) provide one skilled in the art with a general guide to many of the terms used in the present application.

One skilled in the art will recognize many methods and materials similar or equivalent to those described herein, which could be used in the practice of the present invention. Other features and advantages of the invention will become apparent from the following detailed description, taken in conjunction with the accompanying drawings, which illustrate, by way of example, various features of embodiments of the invention. Indeed, the present invention is in no way limited to the methods and materials described. For convenience, certain terms employed herein in the specification, examples and appended claims are collected here.

Unless stated otherwise, or implicit from context, the following terms and phrases include the meanings provided below. Unless explicitly stated otherwise, or apparent from context, the terms and phrases below do not exclude the meaning that the term or phrase has acquired in the art to which it pertains. The definitions are provided to aid in describing particular embodiments, and are not intended to limit the claimed invention, because the scope of the invention is limited only by the claims. Unless otherwise defined, all technical and scientific terms used herein have the same meaning as commonly understood by one of ordinary skill in the art to which this invention belongs.

| SEQUENCE IDENTIFIERS | GENE | ACCESSION NUMBER |
|---|---|---|
| 1 | Human HCN2 | GenBank AF065164 |
| 2 | Human KCNE2 NCBI Reference Sequence: NM_172201.1 | NCBI Reference |
| | | Sequence: |
| | | NM_172201.1 |
| 3 | Rat SkM1 | NCBI Reference |
| | | Sequence: |
| | | NM_013178.1 |
| 4 | Human SkM1 | GenBank: M81758.1 |
| 5 | Mus musculus amino acid sequence for hyperpolarization- | GenBank: AJ225122.1 |
| | activated cation channel HAC1 | |
| 6 | Mus musculus nucleic acid sequence encoding mRNA sequence | GenBank: AJ225122.1 |
| | for hyperpolarization-activated cation channel HAC1 | |
| 7 | amino acid sequence for the protein Homo sapiens | GenBank: AF065164.2 |
| | hyperpolarization-activated, cyclic nucleotide-gated channel 2 | |
| | (HCN2) | |
| 8 | nucleic acid sequence encoding GenBank: AF065164.2 | GenBank: AF065164.2 |
| 9 | amino acid sequence for Rattus norvegicus minK-related peptide | GenBank: AF071003.1 |
| | 1 mRNA, complete cds | |
| 10 | nucleic acid sequence encoding the mRNA | GenBank: AF071003.1 |
| 11 | amino acid sequence for Homo sapiens potassium voltage- | |
| | gated channel, Isk-related family, member 2 (KCNE2) | |
| 12 | nucleic acid sequence encoding NCBI Reference Sequence: | NCBI Reference |
| | NM_172201.1 | Sequence: |
| | | NM_172201.1 |
| 13 | amino acid sequence for Rattus norvegicus sodium channel, | |
| | voltage-gated, type IV, alpha subunit (Scn4a) | |
| 14 | nucleic acid sequence encoding NCBI Reference Sequence: | Reference Sequence: |
| | NM_013178.1 | NM_013178. |
| 15 | amino acid sequence for Homo sapiens skeletal muscle | |
| | voltage-dependent sodium channel alpha subunit (SkM1) | |
| 16 | nucleic acid sequence for the corresponding GenBank: | GenBank: M81758.1 |
| | M81758.1 | |
| 17 | Homo sapiens sodium channel, voltage-gated, type I, alpha | NCBI Reference |
| | subunit (SCN1A), transcript variant 1/NAV 1.1 | Sequence: |
| | | NM_001165963.1 |
| 18 | Homo sapiens sodium channel, voltage-gated, type II, alpha | NCBI Reference |
| | subunit (SCN2A), transcript variant 2/NAV1.2 | Sequence: |
| | | NM_001040142.1 |
| 19 | Homo sapiens sodium channel, voltage-gated, type III, alpha | NCBI Reference |
| | subunit (SCN3A), transcript variant 2/NAV1.3 | Sequence: |
| | | NM_001081676.1 |
| 20 | Homo sapiens sodium channel, voltage-gated, type IX, alpha | NCBI Reference |
| | subunit (SCN9A)/NAV1.7 | Sequence: |
| | | NM_002977.3 |
| 21 | Homo sapiens sodium channel, voltage-gated, type XI, alpha | NCBI Reference |
| | subunit (SCN11A), transcript variant 1/NAV1.9 | Sequence: |
| | | NM_014139.2 |
| 22 | Homo sapiens sodium channel, voltage gated, type VIII, alpha | NCBI Reference |
| | subunit (SCN8A), transcript variant 2/NAV1.6 | Sequence: |
| | | NM_001177984.2 |
| 23 | Homo sapiens hyperpolarization activated cyclic nucleotide- | NCBI Reference |
| | gated potassium channel 2 (HCN2) | Sequence: |
| | | NM_001194.3 |
| 24 | Mus muculus hyperpolarization-activated, cyclic nucleotide- | NCBI Reference |
| | gated K+ 2 (Hcn2) | Sequence: |
| | | NM_008226.2 |
| 25 | Homo sapiens hyperpolarization activated cyclic nucleotide- | NCBI Reference |
| | gated potassium channel 1 (HCN1) | Sequence: |
| | | NM_021072.3 |
| 26 | Mus musculus hyperpolarization-activated, cyclic nucleotide- | NCBI Reference |
| | gated K+ 1 (Hcn1) | Sequence: |
| | | NM_010408.3 |
| 27 | Homo sapiens hyperpolarization activated cyclic nucleotide- | NCBI Reference |
| | gated potassium channel 4 (HCN4) | Sequence: |
| | | NM_005477.2 |
| 28 | Mus musculus hyperpolarization-activated, cyclic nucleotide- | NCBI Reference |
| | gated K+ 4 (Hcn4) | Sequence: |
| | | NM_001081192.1 |

All of the amino acid sequences and nucleic acid sequences associated with the SEQ IDS listed in the table of sequence identifiers above are set forth in the accompanying sequence listing, which was filed together with the application and is part of this application.

“SkM1” (also referred to as SCN4A and Nav 1.4) means skeletal muscle sodium channel or a functional equivalent thereof, including biologically active fragments or variants thereof. Human SkM1 gene has the nucleic acid sequence set forth in GenBank: M81758.1; and the protein SkM1 has the amino acid sequence set forth in GenBank: AAA60554.1. In certain embodiments the sodium channel for use in embodiments of the invention includes any sodium channel that has a depolarized inactivation relation similar to SkM1 channels. More specifically in an embodiment these SkM1-type channels are identified as having a Vh between about 50 mV and 80 mV. Vh is shorthand for the voltage corresponding to the midpoint of the h-infinity relation of the sodium channel; Vh is the parameter used in describing inactivation of Na channels).

As used herein, a “HCN channel” means a hyperpolarization-activated, cyclic nucleotide-gated ion channel responsible for the hyperpolarization-activated cation currents that are directly regulated by cAMP and contribute to pacemaker activity in heart and brain. HCN channels for use in embodiments of the present invention include HCN1, HCN2 and HCN4 and biologically active fragments or variants thereof. Other HCN channels that can be used in embodiments of the invention include any HCN channels that have activation kinetics similar to HCN2 and cAMP responsiveness similar to HCN2, or a biologically active fragment or variant thereof. Such HCN channels can be variants, mutants or chimeras of HCN1, HCN2 or HCN4 or of the other HCN channels.

“HCN1” means the isoform 1 of the hyperpolarization-activated cyclic nucleotide-gated (HCN) channel or a biologically active fragment or variant thereof. HCN1 as used herein includes the naturally occurring isoform.

“HCN2” means the isoform 2 of the hyperpolarization-activated cyclic nucleotide-gated (HCN) channel or a biologically active fragment or variant thereof. HCN2 as used herein includes the naturally occurring isoform.

“HCM4” means the isoform 4 of the hyperpolarization-activated cyclic nucleotide-gated (HCN) channel or a biologically active fragment or variant thereof. HCN4 as used herein includes the naturally occurring isoform.

The term “nucleic acid” as used herein refers to any natural and synthetic linear and sequential arrays of nucleotides and nucleosides, cDNA, genomic DNA, mRNA, oligonucleotides and derivatives thereof. For ease of discussion, such nucleic acids may be collectively referred to herein as “constructs,” “plasmids,” or “vectors.” Representative examples of the nucleic acids of the present invention include viral and non-viral vectors. The term “nucleic acid” further includes modified or derivatized nucleotides.

The term “gene” or “genes” or “coding sequence” or a sequence which “encodes” a particular protein as used herein refer to is a nucleic acid molecule that is transcribed (in the case of DNA) and translated (in the case of mRNA) into a polypeptide in vitro or in vivo when placed under the control of appropriate regulatory sequences. Genes that are not naturally part of a particular organism's genome are referred to as “foreign genes,” “heterologous genes” or “exogenous genes,” and genes that are naturally a part of a particular organism's genome are referred to as “endogenous genes.” The boundaries of the coding sequence of the polypeptide are typically determined by a start codon at the 5′ (i.e., amino) terminus and a translation stop codon at the 3′ (i.e., carboxy) terminus. For the embodiments of the invention a gene can include, but is not limited to, cDNA from mRNA, genomic DNA sequences, even synthetic DNA sequences, a fragment of a coding sequence, or other nucleic acid sequence that is transcribed and translated into a therapeutic HCN or sodium channel.

A transcription termination sequence will usually be located 3′ to the gene sequence. Moreover, a “gene” typically (i) starts with a promoter region containing multiple regulatory elements, possibly including enhancers, for directing transcription of the coding region sequences; (ii) includes coding sequences, which start at the transcriptional start site that is located upstream of the translational start site and ends at the transcriptional stop site, which may be quite a bit downstream of the stop codon (a polyadenylation signal is usually associated with the transcriptional stop site and is located upstream of the transcriptional stop); and (iii) may contain introns and other regulatory sequences to modulate expression and improve stability of the RNA transcript. Still in accordance with the present invention, a “gene” may refer to a sequence encoding a protein, including HCN and sodium channels.

The term “transcription regulatory sequences” as used herein refers to nucleotide sequences that are associated with a gene nucleic acid sequence and which regulate the transcriptional expression of the gene. The transcription regulatory sequences may be isolated and incorporated into a vector nucleic acid to enable regulated transcription in appropriate cells of portions of the vector DNA. In some cases they precede the region of a nucleic acid sequence that is in the region 5′ of the end of a protein coding sequence that may be transcribed into mRNA or within a protein coding region. transcription regulatory sequences refer collectively to promoter regions, polyadenylation signals, transcription termination sequences, upstream regulatory domains, origins of replication, internal ribosome entry sites (“IRES”), enhancers, and the like, which collectively provide for the replication, transcription and translation of a coding sequence in a recipient cell. Not all of these control elements need always be present, so long as the selected coding sequence is capable of being replicated, transcribed and translated in an appropriate host cell.

The term “promoter region” is used herein in its ordinary sense to refer to a nucleotide region including a DNA regulatory sequence, wherein the regulatory sequence is derived from a gene which is capable of binding RNA polymerase and initiating transcription of a downstream (3′-direction) coding sequence.

“Operably linked” refers to an arrangement of transcription regulatory elements wherein the components so described are configured so as to perform their usual function. Thus, control elements operably linked to a coding sequence/gene are capable of effecting the expression of the coding sequence/gene. The control elements need not be contiguous with the coding sequence/gene, so long as they function to direct the expression thereof. Thus, for example, intervening untranslated yet transcribed sequences can be present between a promoter sequence and the coding sequence and the promoter sequence can still be considered “operably linked” to the coding sequence.

The term “expressed” or “expression” as used herein refers to the transcription from a gene to give an RNA nucleic acid molecule at least complementary in part to a region of one of the two nucleic acid strands of the gene. The term “expressed” or “expression” as used herein also refers to the translation from the RNA nucleic acid molecule to give a protein or polypeptide or a portion thereof.

The term “nucleic acid vector” as used herein refers to viral nucleic acid molecules that can be transduced or transfected into target cardiac or stem cells, which vectors replicate independently of, or within, the host cell genome. A nucleic acid such as a gene and regulatory elements can be inserted into a vector by cutting the vector with restriction enzymes and ligating the pieces together.

The term “non-viral vectors” includes vectors derived from bacteriophage nucleic acid, naked DNA and synthetic oligonucleotides such as chemically synthesized DNA or RNA and natural or synthetic single or double stranded plasmids. “Non-viral vectors,” as used herein, refers to synthetic and non-viral gene delivery vectors that typically consist of DNA (usually plasmid DNA produced in bacteria) that may be delivered to the target cell, usually with the aid of a delivery vehicle. Delivery vehicles may be based around lipids including cationic lipids (such as liposomes) that fuse with the cell membrane, releasing the nucleic acid vectors into the cytoplasm of the cell. Alternatively peptides or polymers, dendrimers and peptides may be used to form complexes with the nucleic acid which may condense as well as protect the therapeutic material as it attempts to reach its target destination. These two approaches are combined in the use of LPD complexes which use a polymer to condense the nucleic acid and a lipid coat to aid entry to the cell.

The term “viral vector” as used herein refers to any genetic element, such as a virus or virion, etc., which is capable of replication when associated with the proper control elements and which can transfer gene sequences between cells. Thus, the term includes viral vectors. On or more one regulatory sequence may be operably linked to a nucleotide coding sequence for a protein of interest, or a biologically active fragment or variant thereof, including HCN2 and SkM1 sodium channels. Viral vectors for use in the present invention include adenoviruses, adeno-associated viruses (AAV), retroviruses including lentivirus, Herpes simplex, vaccine viruses and Gemlike Forest virus.

“Regulatory sequences” includes promoters, enhancers, and other elements that may control expression of a gene. Standard molecular biology textbooks, such as Sambrook et al. eds., “Molecular Cloning: A Laboratory Manual” 2nd ed., Cold Spring Harbor Press (1989), may be consulted to design suitable expression vectors, promoters, and other expression control elements. It should be recognized, however, that the choice of a suitable expression vector depends upon multiple factors including the choice of the host cell to be transformed and/or the type of protein to be expressed.

“rAAV vector” and “AAV” are used interchangeably to refer to any vector derived from any adeno-associated virus serotype, including, without limitation, AAV-1, AAV-2, AAV-3, AAV-4, AAV-5, AAV-7 and AAV-8, and the like. rAAV vectors can have one or more of the AAV wild-type genes deleted in whole or in part, preferably the Rep and/or Cap genes, but retain functional flanking ITR sequences. Functional ITR sequences are generally necessary for the rescue, replication, packaging and potential chromosomal integration of the AAV genome. Thus, a rAAV vector is defined herein to include at least those sequences required in cis for replication and packaging (e.g., functional ITRs) of the virus. The ITRs need not be the wild-type nucleotide sequences, and may be altered (e.g., by the insertion, deletion or substitution of nucleotides) so long as the sequences provide for functional rescue, replication and packaging. AAV vectors rather than adenovirus vectors AV are the most likely therapeutic candidate for humans.

“AAV biological pacemaker construct” and “AAV-BP construct” as used herein refer to a AAV/rAAV vector that carries a gene for an enumerated HCN channel or an enumerated SkM1 channel or both for use in embodiments of the present invention.

“Biological pacemaker constructs” and “BP-constructs” as used herein refers generally to any viral construct or non-viral construct that comprises a gene for an enumerated HCN channel or an enumerated sodium channel or both for use in embodiments of the present invention. These constructs typically include a cardiac specific or constitutive promoter as described herein. BP constructs include adenoviral and AAV BP constructs.

“Recombinant virus” refers to a virus that has been genetically altered (e.g., by the addition or insertion of a heterologous nucleic acid construct, such as a gene for an HCN of SkM1 channel, into the particle).

“AAV virion” refers to a complete virus particle, such as a wild-type (“wt”) AAV virus particle (i.e., including a linear, single-stranded AAV nucleic acid genome associated with an AAV capsid protein coat). In this regard, single-stranded AAV nucleic acid molecules of either complementary sense (i.e., “sense” or “antisense” strands) can be packaged into any one AAV virion; both strands are equally infectious. In addition, the AAV capsid protein coat can be from any of the various AAV serotypes depending on the target of the AAV virion.

A “recombinant AAV virion” or “rAAV virion” is defined herein as an infectious, replication-defective virus composed of an AAV protein shell, encapsidating a heterologous DNA molecule of interest (e.g., genes encoding caspase-1) which is flanked on both sides by AAV ITRs. A rAAV virion may be produced in a suitable host cell which has had an rAAV vector, AAV Rep and Cap functions and helper virus functions introduced therein. In this manner, the host cell is rendered capable of producing AAV replication and capsid proteins that are required for replicating and packaging the rAAV vector (i.e., containing a recombinant nucleotide sequence of interest) into recombinant virion particles for subsequent gene delivery. The complete gene added to a vector may consist of a promoter, the coding sequences, usually a cDNA and a polyadenylation signal. A gene may also include regulatory sequences and intron regions. Promoters that would regulate transgene expression may include constitutive, inducible and tissue-specific promoters.

“Transduction” is generally used to designate viral gene delivery to a recipient cell either in vivo or in vitro, via any method of gene delivery, including replication-defective viral vectors, such as via a rAAV. For example, transduction could mean uptake of a virus, part of which is DNA. “Transfection” generally designates non-viral gene delivery. However, the terms “transfection” and “transduction” are used interchangeably herein to refer to the process of inserting a nucleic acid viral vector into a host (i.e. in vivo administration for example as described herein to the heart) or to a cell in vitro.

By “administering” the vector constructs is meant injecting or otherwise delivering the viral or non-viral vector constructs of embodiments of the invention to a subject by a method or route which results in at least partial localization of the vectors in the targeted area of the heart. Administration can be endovascular via infusing biological pacemaker constructs into the coronary vasculature, but is preferably via direct injection into the heart, intramyocardially (intramuscularly) or by infusion into the coronary vasculature. Vectors or cells are administered in saline or any other solution which may be used to enhance viral transduction, cell homing or cell survival.

As used herein, the terms “treat,” “treatment,” “treating,” or “amelioration” when used in reference to a disease, disorder or medical condition, such as sinoatrial node dysfunction and/or atrioventricular conduction block refer to therapeutic treatments for a condition, wherein the object is to reverse, alleviate, ameliorate, inhibit, slow down or stop the progression or severity of a symptom or condition. The term “treating” includes reducing or alleviating at least one adverse effect or symptom of a condition. Treatment is generally “effective” if one or more symptoms or clinical markers are reduced. Alternatively, treatment is “effective” if the progression of a disease-state is reduced or halted. That is, “treatment” includes not just the improvement of symptoms or markers, but also a cessation or at least slowing of progress or worsening of symptoms that would be expected in the absence of treatment. In the context of the present invention, the treatment will provide cardiac pacing in a subject that has sinoatrial node dysfunction and/or atrioventricular conduction block.

The term “biologically active,” when used in conjunction with “derivative” or “variant” or “fragment” or “mutant” form of a channel, refers to a polypeptide which possess a biological activity that is substantially similar to or has a greater biological activity than the entity or molecule of which it is a derivative or variant or fragment or mutant thereof.

A “subject” can be one who has been previously diagnosed with or identified as suffering from sinoatrial node dysfunction and/or atrioventricular conduction block. As used herein, a “subject” means an animal, preferably a mammal, most preferably a human.

“Mammal” as used herein refers to any member of the class Mammalia, including, without limitation, humans and nonhuman primates such as chimpanzees and other apes and monkey species; farm animals such as cattle, sheep, pigs, goats and horses; domestic mammals such as dogs and cats; laboratory animals including rodents such as mice, rats and guinea pigs, and the like. The term does not denote a particular age or sex. In addition, the methods described herein can be used to treat domesticated animals and/or pets.

The term “statistically significant” or “significantly” refers to statistical evidence that there is a difference. It is defined as the probability of making a decision to reject the null hypothesis when the null hypothesis is actually true. The decision is often made using the p-value.

As used herein, “variants” can include, but are not limited to, those that include conservative amino acid mutations, SNP variants, splicing variants, degenerate variants, and biologically active portions of a gene. A “degenerate variant” as used herein refers to a variant that has a mutated nucleotide sequence, but still encodes the same polypeptide due to the redundancy of the genetic code. In accordance with the present invention, the enumerated HCN2 and SkM1 genes may be modified, for example, to facilitate identification and/or improve expression, so long as such modifications do not reduce function to an unacceptable level. In various embodiments, a variant of the gene encodes a protein that has at least 10%, 20%, 30%, 40%, 50%, 60%, 70%, 80%, 90%, or 95% of the function of a wild-type protein.

For the purpose of describing the relative position of nucleotide sequences in a particular nucleic acid molecule throughout the instant application, such as when a particular nucleotide sequence is described as being situated “upstream,” “downstream,” “5′,” or “3′” relative to another sequence, it is to be understood that it is the position of the sequences in the non-transcribed strand of a DNA molecule that is being referred to as is conventional in the art.

“Homology” and “homologous” as used herein refer to the percent of identity between two polynucleotide and two polypeptide moieties. The correspondence between the sequence from one moiety to another can be determined by techniques known in the art. For example, homology can be determined by a direct comparison of the sequence information between two polypeptide molecules by aligning the sequence information and using readily available computer programs. Alternatively, homology can be determined by hybridization of polynucleotides under conditions which form stable duplexes between homologous regions, followed by digestion with single-stranded-specific nuclease(s), and size determination of the digested fragments. Two DNA or two polypeptide sequences are “substantially homologous” to each other when at least about 80%, preferably at least about 90%, and most preferably at least about 95% of the nucleotides or amino acids, respectively, match over a defined length of the molecules, as determined using the methods above.

“Isolated” as used herein when referring to a nucleotide sequence, vector, etc., refers to the fact that the indicated molecule is present in the substantial absence of other biological macromolecules of the same type. Thus, an “isolated nucleic acid molecule which encodes a particular polypeptide” refers to a nucleic acid molecule that is substantially free of other nucleic acid molecules that do not encode the subject polypeptide. Likewise, an “isolated vector” refers to a vector that is substantially free of other vectors that differ from the subject vector. However, the subject molecule or vector may include some additional bases or moieties that do not deleteriously affect the basic characteristics of the composition.

“Purified” as used herein when referring to a vector, refers to a quantity of the indicated vector that is present in the substantial absence of other biological macromolecules. Thus, a “purified vector” refers to a composition that includes at least 80% subject vector, preferably at least 90% subject vector, most preferably at least 95% subject vector with respect to other components of the composition.

### DETAILED DESCRIPTION

It has been discovered that hyperpolarization-activated cyclic nucleotide-gated (HCN)-based biological pacing, especially that achieved with transduction of the HCN2 gene into cardiac cells in vivo, was significantly improved by hyperpolarizing the action potential (AP) threshold via coexpression via co-transduction of the skeletal muscle sodium channel 1 (SkM1) gene. When adenoviral-biological pacemaker constructs (AV-BP constructs) carrying genes for HCN2 and SkM1 were administered to the into left bundle branches (LBB) of dogs in vivo, pacemaker function was facilitated by the slow depolarizing HCN2 current and the hyperpolarized AP threshold generated by SkM1. This dual gene therapy provided both highly efficient pacing and a brisk autonomic response that is superior to those of previously developed gene- or cell-based approaches.

The combination of the pacemaker gene HCN2 and the skeletal muscle sodium channel, SkM1, has provided the most potentially clinically applicable of any of the biological pacemaker strategies studied so far. For the first time, biological pacemaker rhythms are generated in more than 95% of the time, in a large animal model comparable to patients requiring a ventricular demand pacemaker. Baseline beating rates are within the target range of 70-80 bpm and demonstrate a brisk autonomic response.

Based on the results described in the examples, certain embodiments of the present invention provide methods for treating sinoatrial node dysfunction and/or atrioventricular conduction block, which is sometimes referred to as treating cardiac pacing or conduction dysfunction that has an electrical malfunction, in a subject, by administering to an area of the heart at, near or remote from the site of the sinoatrial node dysfunction and/or atrioventricular conduction block, a composition comprising one or more viral vector constructs for delivering a gene encoding a biologically active hyperpolarization-activated cyclic nucleotide-gated (HCN) channel, or a biologically active fragment or variant thereof, such as HCN2, and a gene encoding a biologically active voltage gated sodium channel or a biologically active fragment or variant thereof, such as SkM1. The vectors are administered in a therapeutically effective amount so that the two genes are coexpressed in a transduced plurality of cells at or near the area thereby treating the sinoatrial node dysfunction and/or atrioventricular conduction block. The genes are typically in different vectors but can be in the same vector.

In some embodiments the HCN gene encodes an HCN protein that confers on the cell a biological activity similar to the HCN 2 with activation kinetics and cAMP responsiveness that are similar to HCN2 channels. In some embodiments the HCN gene is HCN1 or HCN4 or biologically active fragments or variants thereof. In some embodiments the sodium channel is selected to include a sodium channel that has a depolarized inactivation relation similar to SkM1 channels (which is also called Nav 1.4). In some embodiments specific examples of Na channels are extended to include the “neuronal” isoforms including (Nav 1.1, 1.2, 1.3, 1.6, 1.7 and 1.9) and any other non-cardiac isoforms that have midpoints of their inactivation relation (Vh values) similar to SkM1.

In some embodiments the HCN gene encodes an HCN protein that confers on the cell a biological activity similar to the HCN 2 with activation kinetics and cAMP responsiveness that are similar to HCN2 channels. In some embodiments the HCN gene is HCN2 or HCN4. In certain embodiments the sodium channel is selected to include a sodium channel that has a depolarized inactivation relation (Vh is shorthand for the voltage corresponding to the midpoint of the h-infinity relation of the sodium channel; h is the parameter used in describing inactivation of Na channels) similar to SkM1 channels (which is also called Nav 1.4). In some embodiments sodium channels include the “neuronal” isoforms (NAV 1.1, 1.2, 1.3, 1.6, 1.7 and 1.9) and any other non-cardiac isoforms that have midpoints of their inactivation relation (Vh values) similar to SkM1 (Nav 1.4) or in a range of from about 80 mV to about 50 mV, which is the Vh for SkM1.

The viral constructs for use in embodiments of the invention can be made from any vector, including an adenoviral AV vectors as was used in the examples (preferably “gutless” adenoviral vectors), an adeno-associated viral (AAV), and a retroviral vector such as a lentiviral vector. A lentivirus vector is able to carry large genes and can survive for years, possibly indefinitely. The gene for full length SkM1 is about 5 kb in size and therefore requires a viral vector that can accommodate this large a protein. Alternatively the gene for SkM1 could be administered in a dual vector as described herein (47). Once in the target cell, the viral vector can be extragenomic or intragenomic. An extragenomic nucleic acid vector does not insert into the cell's genome. The term intragenomic defines a nucleic acid construct incorporated within the transduced cell's genome. When expressing HCN or sodium channels in stem cells either viral delivery or standard transfection techniques such as electroporation are used.

Other embodiments of the present invention provide cardiac cells or other cells such as hMSC or other stem cell that have been genetically engineered to express an enumerated HCN channel and sodium channel.

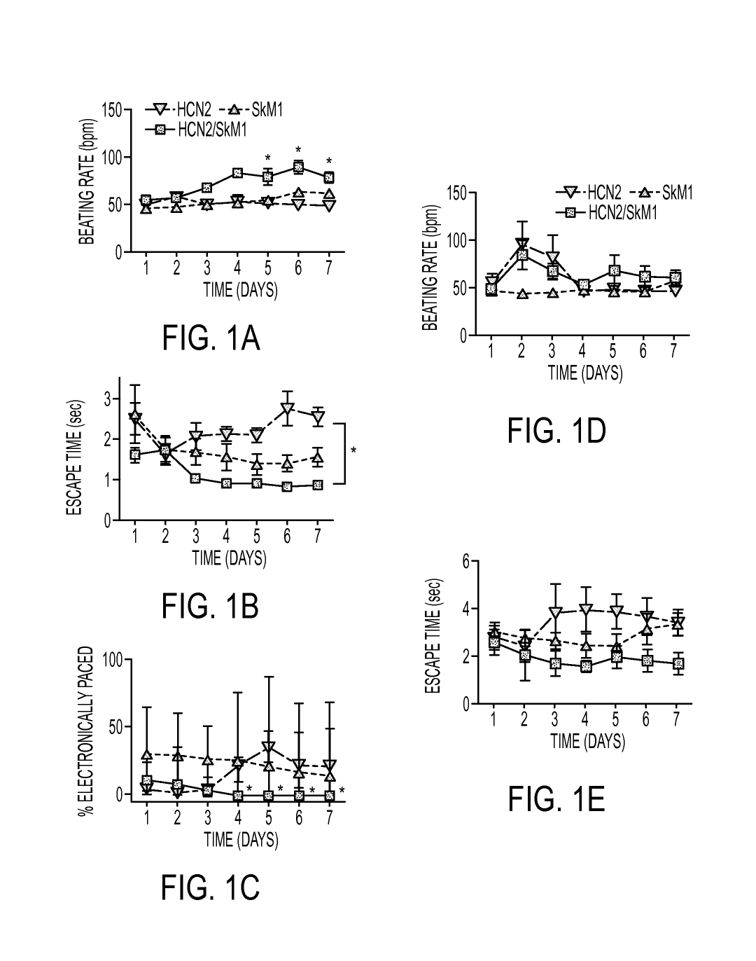

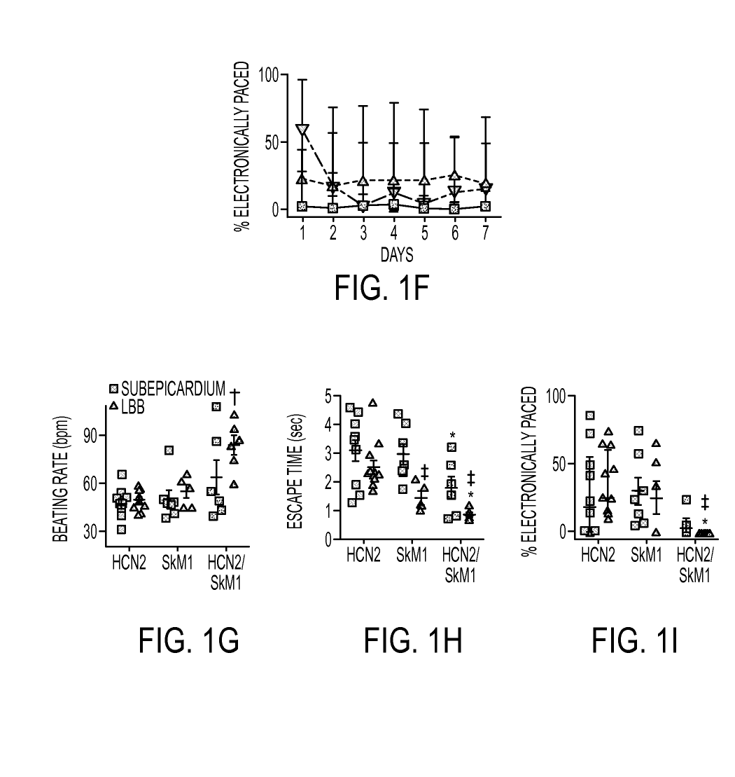

The targeted area(s) of the subject's heart can be the area that is malfunctioning in a subject with sinoatrial node dysfunction and/or atrioventricular conduction block, an area nearby or an area that is remote and that functions normally; e.g., if the AV-node is dysfunctional, non-diseased or diseased purkinje fibers (that are nearby or remote to the AV-node) may be used to generate biological pacemaker function after HCN2/SkM1 gene transfer, as was done in the experiment summarized in FIG. 1. In case of SAN dysfunction, HCN2/SkM1 gene transfer may be applied to non-diseased or diseased nodal tissue, non-diseased or diseased nearby right atrial tissue, or non-diseased or diseased remote left atrial tissue. Depending on the type of biological pacemaker construct, gene and the heart condition, it may take from days to weeks after administration before transduced cells (in vitro) express their pacing functions.

In some embodiments, the BP constructs of the invention carry genes (HCN and sodium channels) that are selected to achieve different pacing rates, depending on the subject's condition and the injection site. For example, by controlling the gene expression one can regulate the type or amount of encoded polynucleotide expressed in different heart regions. In some embodiments different BP constructs may be administered to the subject in different locations with each producing a unique pacing rate to decrease the likelihood of simultaneous activity originating from any other distinct idioventricular pacemaker sites native to the individual. Thus, AV or AAV constructs may be introduced into any desired heart region to treat a particular arrhythmia, while mitigating the risk of the overall system being arrhythmogenic.

In some experiments hMSCs loaded with HCN2 and SkM1 AV constructs were injected into the left ventricular anterior free wall epicardium, a procedure previously shown to provide good pacemaker function when hMSCs loaded with HCN2 alone are injected. The results showed only poor biological pacemaker function (data not shown), an outcome entirely different from the robust function that reported herein when the adenoviral vectors carrying the HCN2 and SkM1 genes were injected into the heart. However, while not yet perfected, injection of HCN2- and SkM1-transfected hMSCs may be useful in the future for pacemaker therapy of sinoatrial node dysfunction and/or atrioventricular conduction block.

### Overview

Previous work (Rosen application Pub. No. 20040137621) was aimed at speeding conduction to reduce arrhythmia vulnerability or to repair a non-conducting AV-node, where it was an advantage to increase conduction, and therefore connexin expression was needed. By contrast, when treating the sinoatrial node dysfunction and/or atrioventricular conduction block using the injected BP constructs of the present invention, it is desirable to reduce the electrical load to enhance pacemaker robustness—therefore in some embodiments the expression or function of fast-conducting connexins (in atria Cx40&43, in ventricle Cx43) is reduced as described in Boink et al., WO/2009/12008, incorporated herein by reference. In some embodiments the fast-conducting connexions are reduced using antisense or small interfering RNAs. In other embodiments a compound capable of reducing the amount and/or activity of gap junction proteins connecting the cell and surrounding cells, a gap junction protein with a diminished conductor capacity as compared to connexin 43, a gap junction protein with a diminished conductor capacity as compared to connexin 40, a compound capable of reducing the amount and/or activity of connexin 43 of the cell, a compound capable of reducing the amount and/or activity of connexin 40 of the cell, a connexin with a lower conductor capacity than the conductor capacity of connexin 43, a connexin with a lower conductor capacity than the conductor capacity of connexin 40, connexin 30.2 or a functional equivalent thereof, connexin 45 or a functional equivalent thereof, connexin 43Δ or a functional equivalent thereof, a transcription factor capable of reducing connexin 43 expression, a transcription factor capable of reducing connexin 40 expression and TBX3 or a functional equivalent thereof.

Electronic cardiac pacing provides effective treatment for heart block and/or sinus node dysfunction but has shortcomings, including inadequate autonomic modulation, limited battery life, lead fracture, and an association with potentially deleterious cardiac remodeling (1). In seeking better alternatives, diverse strategies to create biological pacemakers have been explored (1). These strategies have used spontaneously active cells (e.g., derivatives of embryonic stem cells or induced pluripotent stem cells) or pacemaker function-related genes delivered via cell platforms or viral vectors.

Recent efforts have focused on improving gene-based biological pacemakers. However, engineered hyperpolarization-activated cyclic nucleotide-gated (HCN) variants have provided improvements that are subtle (e.g., HCN2, E324A) (2) or excessive (e.g., HCN212) (3). Alternative strategies have included overexpressing calcium-stimulated adenylyl cyclase (AC1) (4) or the dominant negative inward rectifier channel Kir2.1AAA (5), and the combination strategies of HCN2/AC1 (4) or HCN2/Kir2.1AAA (5).

Outcomes for baseline and maximal rates of in vivo LBB-implanted HCN2/SkM1-based biological pacemakers in dogs as described in the Examples below compared favorably with results reported for AC1 and various HCN isoforms, mutants, and gene combinations. Rates with AC1 (4), wild-type HCN2, and genetically engineered HCN2-E324A, were consistently slower than with HCN2/SkM1 (2). Although the HCN2/Kir2.1AAA strategy generated robust pacemaker activity at relatively rapid baseline beating rates (90 to 95 beats/min), dependence on electronic backup pacing was not eliminated (5). Moreover, the response to autonomic modulation in HCN2/Kir2.1AAA appeared inferior compared to HCN2/SkM1. Regardless, some embodiments include replacing the sodium channel gene in the present methods with the Kir2.1AAA gene. Although the AC1 strategy shows promise with respect to the high efficiency of pacemaker function (95% of the beats originated from the injection site), physiological beating rates (approximately 60 beats/min), and high sensitivity to parasympathetic modulation, it also manifested relatively slow maximal beating rates and did not eliminate electronic backup pacing (4). Moreover, the AC1 strategy elevated cAMP levels and impacted calcium handling in cells (10-12), presenting the potential for unwanted side effects such as triggered activity and calcium overload.

In contrast, the present HCN2/SkM1 gene combination induced baseline and maximal beating rates with optimal target ranges for a biological pacemaker. Furthermore, calcium overload, which could conceivably occur with a sodium channel construct, was not an issue here (6). Finally, favorable pacemaker function as manifested by short escape times and low-to-absent dependence on electronic backup pacing was also characteristic of HCN2/SkM1 LBB injected animals.

### Results

Pre-defined optimal outcomes for a biological pacemaker include:

1) basal beating rates of 60 to 90 beats/min;

2) autonomic responsiveness resulting in rate increases to 130 to 160 beats/min; and

3) low to absent dependence on electronic backup pacing (4,5).

To achieve these optimal parameters, cardiac cells were transduced with adenoviral biological pacemaker constructs (AV-BP constructs) that achieved coexpression of biologically active skeletal muscle sodium channel 1 (SkM1) and HCN channel 2 (HCN2). The rationale was as follows: HCN channels generate inward current that drives the membrane toward threshold for the rapid inward sodium current (INa). To reach the threshold for INa, channel opening could be maximized. Previous work showed that the SkM1 sodium channel has a more depolarized inactivation versus voltage curve and more rapid recovery kinetics from inactivation than the cardiac sodium channel SCN5A (6,7). Thus, SkM1 was expected to provide greater availability of sodium channels during diastole, leading to a more negative threshold potential, improved pacemaker stability, and increased beating rates. The results herein show that this was the result of transducing cardiac cells with BP constructs to facilitate expression of HCN2 and SkM2 channels.

An example of a dual HCN/Sodium channel transduction using two separate AV-BP constructs is described in detail in Example 1. The biological pacemaker robustness of adenoviral HCN2-SkM1 constructs delivered into the left bundle branch (LBB) of the canine ventricular conducting system in vivo was investigated and compared to transduction with HCN2 or SkM1 alone. The results showed that the adenoviral dual HCN2-SkM1-transducted cells developed biological pacemaker activity that facilitated normal beating rates into the 70 bpm range and complete abolition of pacemaker instabilities (generating function more than 95% of the time), together with brisk autonomic modulation within 48 hours of injection. The data therefore confirm that the physiological characteristics of pacemaker function were influenced by the both the HCN2 and SkM1 channels, indicating that both genes were successfully transduced and expressed in the ventricular conducting system when injected in vivo to the ventricular conducting system.

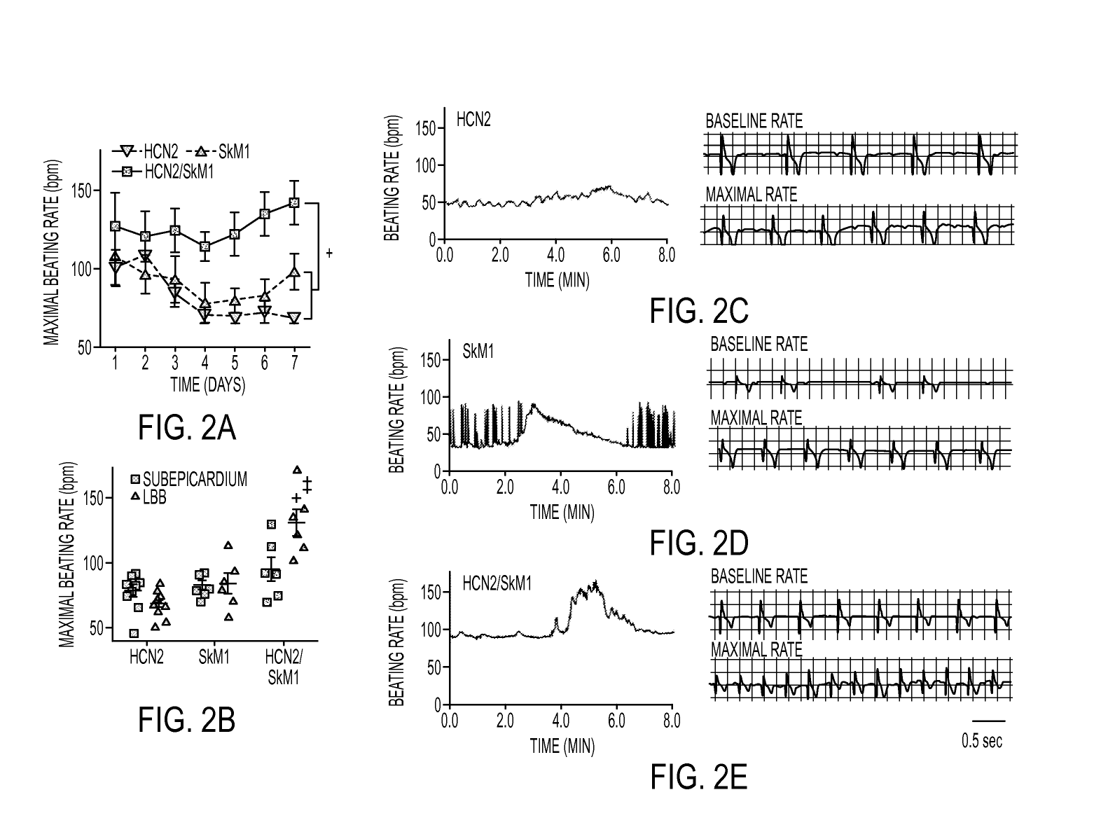

FIG. 1 provides summary data for left bundle block (LBB)-injected animals receiving erpolarization-activated cyclic nucleotide-gated channel 2 (HCN2; n=12), skeletal muscle sodium channel 1 (SkM1; n 5 in A and B, n 6 in C), or both HCN2/SkM1 (n=6). That baseline beating rates on days 3 to 7 were faster in HCN2/SkM1-injected animals than in animals injected with only HCN2 or SkM1, and the mean escape times on days 4 to 7 were shorter in HCN2/SkM1-injected animals than HCN2-injected animals. Further, the median percentage of electronically stimulated beats calculated over 24-h periods was significantly lower in HCN2/SkM1-injected animals than animals injected with HCN2 alone. Importantly, on days 4 to 7, electronic backup pacing was eliminated in HCN2/SkM1-injected animals. Faster beating rates were reached in HCN2/SkM1 LBB-injected animals than those injected with HCN2 or SkM1 (FIG. 2).

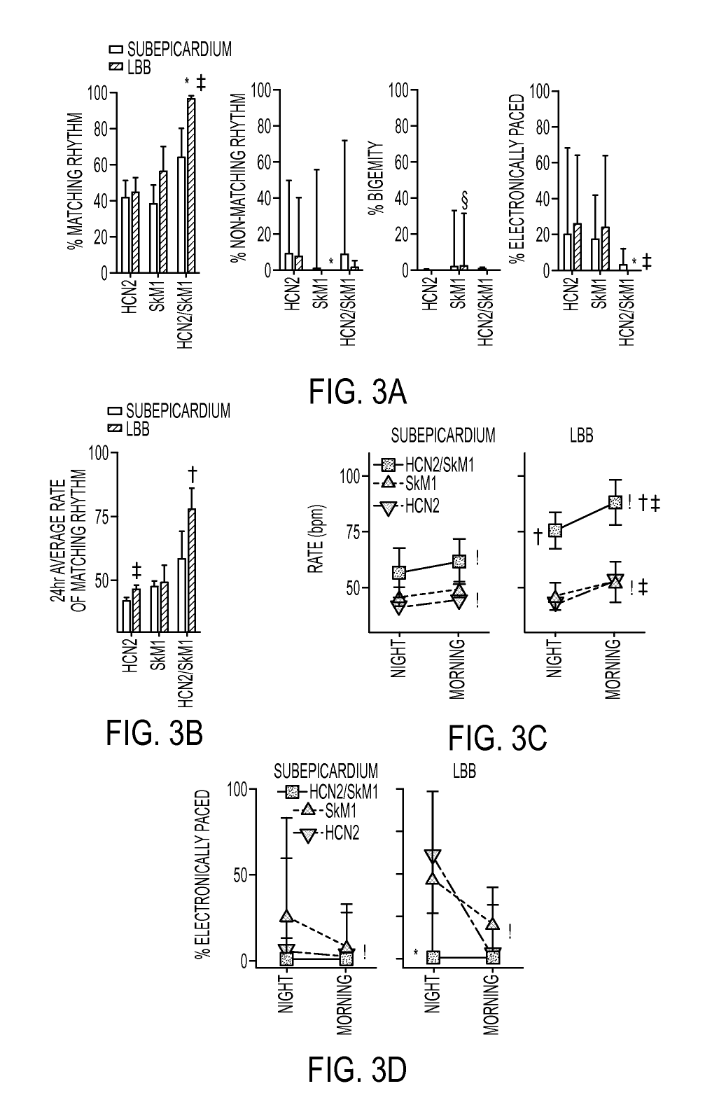

The percentage of matching pace-mapped beats was significantly higher in HCN2/SkM1 LBB-injected animals (95% of all beats), requiring less pacemaker backup than the respective HCN2- and SkM1-injected groups (p less than 0.05). The percentage of matching beats in animals that received HCN2/SkM1 into subepicardium was lower (approximately 60%) and did not differ from that of HCN2 and SkM1 control groups (FIG. 3). Thus, the area for administering the viral or non-viral constructs to the heart of a subject that will achieve optimal results should be determined on a case by case basis. An electrophysiological study is performed for each subject, in which multiple left and/or right ventricular sites are stimulated sequentially. The site that gives the optimal cardiac output, which may or may not be the LBB, is the area where the compositions comprising the described viral are administered; administration to additional sites is also an embodiment.

The results discussed in the examples further showed that there was no dependence on electronic backup pacing, and enhanced modulation of pacemaker function during circadian rhythm or epinephrine infusion in the HCN2/SkM1-LBB group. Studies on the average beating rates comparing a period of rest (2:00 to 4:00 AM) with one of physical activity and feeding (8:00 to 10:00 AM) showed that beating rates in accordance with those expected with a normal response to circadian modulation. Further, the circadian response in the HCA2/SkM1-LBB group was superior to that in animals with LBB gene transfer of HCN2 or SkM1 and myocardial gene transfer of HCN2/SkM1 (FIG. 3).

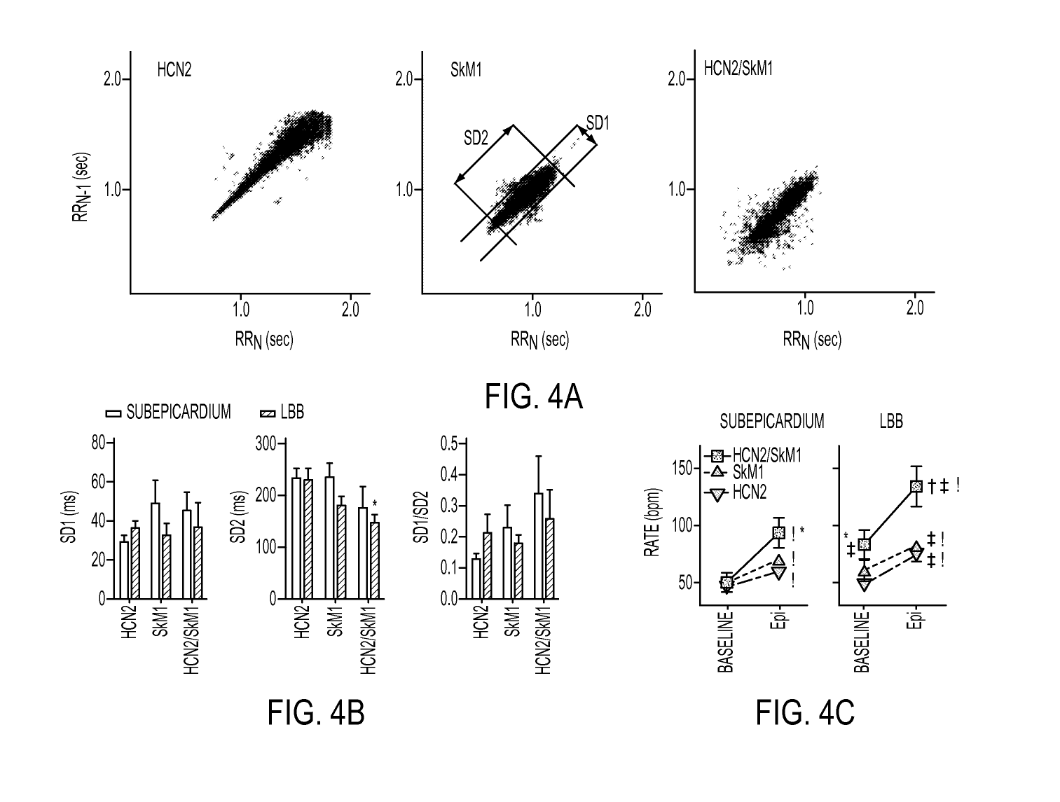

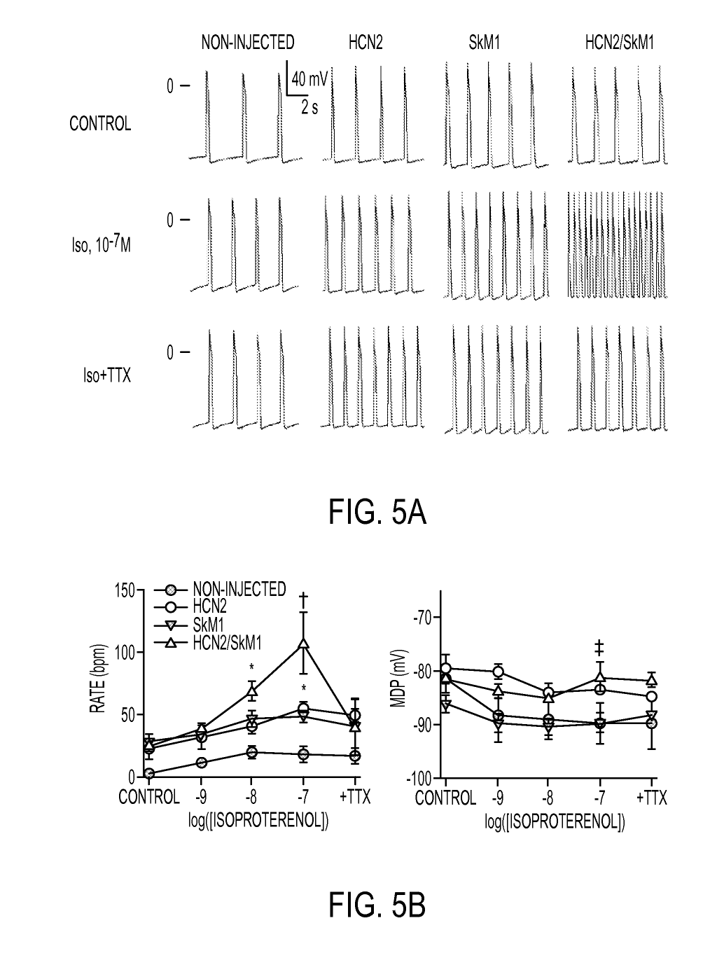

Sensitivity to parasympathetic and sympathetic modulation was studied via analysis of and infusion of epinephrine which showed that the lower average values found for SD2 in the HCN2/SkM1-LBB group were due to reduced activity of the sympathetic system during rest rather than reduced sensitivity to sympathetic modulation (FIGS. 4 and 5).

Finally, in isolated tissue studies, data acquired from the first 9 action potentials (APs) per cycle that were stimulated normally confirmed the functional presence of SkM1 in the SkM1 and HCN2/SkM1 groups (data not shown). Specifically, SkM1 overexpression induced an increase in maximal upstroke velocity in the SkM1 and HCN2/SkM1 groups compared with those in the respective noninjected controls and the HCN2-injected group (p<0.05) (22).

### HCN Channels

All four isoforms are expressed in brain; HCN1, HCN2 and HCN4 are also prominently expressed in heart, with HCN4 and HCN1/2 (depending on species) predominating in sinoatrial node and HCN2 in the ventricular specialized conducting system. “mHCN” designates murine or mouse HCN; “hHCN” designates human HCN.

HCN channels, similar to voltage-gated K+(Kv) channels, have four subunits, each of which has six transmembrane segments, S1-S6: the positively charged S4 domain forms the major voltage sensor, whereas S5 and S6, together with the S5-S6 linker connecting the two, form the pore domain containing the ion permeation pathway and the gates that control the flow of ions. Mutational studies on HCN channels indicate that mutations in the S4 voltage sensor, the S4-S5 linker implicated in the coupling of voltage sensing to pore opening and closing, the S5, S6 and S5-S6 linker which form the pore, the C-linker, and the C-terminal cyclic nucleotide binding domain (CNBD), may be particularly important in affecting HCN channel activity. In some embodiments a mutant HCN channel may be used to provide an improved characteristic, as compared to a wild-type HCN channel, such as faster kinetics, more positive activation, increased expression, increased stability, preserved or enhanced cAMP responsiveness, and preserved or enhanced neurohumoral response. Mutant HCN channels for inducing pacemaker activity in cells is also described in U.S. Provisional Application No. 60/781,723 (filed Mar. 14, 2006) and is discussed in U.S. application Ser. No. 10/342,506. In certain embodiments of the present invention, the mutant HCN channel carries at least one mutation in S4 voltage sensor, the S4-S5 linker, S5, S6, the S5-S6 linker, and/or the C-linker, and the CNBD which mutations result in one or more of the above discussed improved characteristics. In other embodiments, the HCN mutant is identified in the art as E324A-HCN2, Y331A-HCN2, R339A-HCN2, or Y331A, E324A-HCN2. In preferred embodiments, the mutant HCN channel is E324A-HCN2. Other mutations of the HCN channel are set forth in U.S. Ser. No. 11/490,760. Certain embodiments include the HCN1 mutant to the claims described in Tse et al. (50).

Some HCN chimeras have been described that provide an improved characteristic, as compared to a wild-type HCN channel, such as faster kinetics, more positive activation, increased expression, increased stability, preserved or enhanced cAMP responsiveness, and preserved or enhanced neurohumoral response. These as well as mutant HCN channels with beneficial characteristics with respect to cardiac pacemaking and conduction can also be used in embodiments of the present invention. HCN chimeras for inducing pacemaker activity in cells are described in detail in U.S. Provisional Application No. 60/715,934 (filed Sep. 9, 2005) and in U.S. patent application 20070099268, Ser. No. 11/519,399. In an embodiment the HCN channel is HCN2-DM.

As used herein, a “HCN chimera” or “chimeric HCN channel” shall mean a HCN channel comprising portions of more than one HCN channel isoform. Thus, a chimera may comprise portions of HCN1 and HCN2 or HCN3 or HCN4, and so forth. In preferred embodiments, the portions are an amino terminal portion, an intramembranous portion, and a carboxy terminal portion. In preferred embodiments, the portions are derived from human HCN isoforms.

In some embodiments the pacemaker activity of a HCN channel may be enhanced by co-expressing it with its beta subunit, MiRP1, which increases the magnitude of the current expressed and/or speeds its kinetics of activation. See U.S. Pat. No. 6,783,979 and Qu et al. (2004), the entire contents of which are incorporated herein by reference. The pacemaker current is conducted by electrotonic conduction. In other embodiments, the pacemaker current is actively propagated by an action potential. In various embodiments, the pacemaker ion channel HCN further includes both the alpha and the MiRP1 beta subunit.

In certain embodiments the biological pacemakers may improved by modifying basal and β-adrenergic stimulated cAMP levels, for example with additional overexpression of adenylyl cyclase (AC) genes. Additional gene modifications are easily introduced into the BP using routine methods known in the art. In certain embodiments the vectors may additionally overexpress AC1; this is a Ca²⁺-stimulated AC, expressed at high levels in the SAN. Expression of AC1 has been linked to elevated cAMP levels in SAN cells, and is expected to contribute to enhanced Ca²⁺-cycling, an important mechanism in SAN pacing. It has already been demonstrated that adenoviral AC1 delivery together with HCN2 into the left bundle branch of the dog provides an efficient strategy to increase basal and maximal pace-mapped beating rates.

### Biological Pacemaker Vectors

Adenoviral constructs of green fluorescent protein (GFP), mouse HCN2 and rat SkM1, all driven by the CMV promoter were used in the examples herein and were prepared as described previously (1, 2). An empty adenoviral vector was prepared as an additional control vector. For consistency with earlier studies (3), 3×10¹⁰ fluorescence focus forming units of one vector were prepared and mixed with an equal amount of the other vector in a total volume of 700 μL. Because the SkM1 gene (5.4 kb) was too large to combine in a single adenoviral vector known at the time the experiments were done, a separate AV construct was made for the HCN and SkM1 genes. AAV vectors that can accommodate up to 6 kb gene sequence are now available. Further description of making the AV vectors is found in references 25 and 26.

Gene therapy uses designated vehicles to deliver genetic information to specific target cells. In the heart, such targeting may be achieved by injecting vectors into specific areas or infusing them into the coronary vasculature. Multiple viral vectors have been considered for gene delivery to the heart, and the key characteristics of each are summarized in Table 1 below Adenoviral vectors are useful candidates because they are easily produced, transduce myocardium efficiently, and have a large insert capacity allowing investigation of a broad range of candidate genes. However, because the gene is not integrated into the host genome gene expression generally only persists for weeks (27, 28). Therefore, these vectors are suitable for proof-of-concept studies but their clinical value is limited.

TABLE 1

| | Adenovirus | AAV | Lentivirus |
|---|---|---|---|
| Viral Genome | dsDNA | SS or ds DNA | RNA |
| Cloning capacity | 7.9 kb | 6 kb | 8.0 kb |
| Vector genome | episomal | ~90% episomal | Integrated |
| | | ~10 integrated | |
| | | Long term gene | |
| | | expression of | |
| | | small genes | |
| Major area of | Short term gene | | Long term gene |
| application | expression & | | expression of |
| | proof-of- | | small to large |
| | principle | | genes & |
| | studies | | ex vivo |
| | | | modification of |
| | | | stem cells |

Long-term myocardial gene transfer is typically generated by adeno-associated viral vectors (AAVs). Because all the viral genes have been removed from this vector, they only induce a minimal inflammatory response. Moreover, myocardial gene transfer has been shown to persist for at least 12 months (29), and in a recent study as long as 31 months (30); studies outside the heart support the expectation for long duration of transgene expression (31). The AAV-based gene transfer has also been found safe in clinical testing of patients with heart failure (32). At this stage, AAV therefore provides a useful platform to develop novel gene therapies for the heart. Because AAV-based gene transfer remains largely episomal, it is uncertain whether expression will be maintained over the longer term (5-10 years), however, the procedure can be repeated as needed using the methods of the embodiments described herein.

Alternatively, other systems such as lentiviral vectors can be used in embodiments of the invention. Lentiviral vectors are derived from the human immunodeficiency virus and, similar to this virus, they integrate into the host genome (34). They efficiently transduce cardiac myocytes (35, 36) and probably have the best potential for very long-term gene expression. Lentiviral vectors are now primarily used clinically for the ex vivo modification of stem cells (37). The first clinical trials using in vivo lentiviral gene transfer targeting the immune system, the eye and the brain (38-41) have been initiated, and it appears likely that, over time, these studies will extend to other organs including the heart.

The type of viral vector selected is dependent on the target tissue and the length of the sequence to be delivered. For a discussion of viral vectors see Gene Transfer and Expression Protocols, Murray ed., pp. 109-206 (1991). The above-mentioned delivery systems and protocols therefore are described in Kmeic, Gene Targeting Protocols, 2nd ed., pp. 1-35 (2002), and Murray ed., Gene Transfer and Expression Protocols, Vol. 7, pp 81-89 (1991).

Lentiviral vectors have the advantage of being able to harbor large genes such as full-length SkM1, and their integration into the host genome induces long-term function therefore they lentiviral constructs can be designed that carry the HCN/S odium channel genes such as HCN2/SkM1, plus additional elements to improve production (e.g. insulators or doxycycline sensitive promoters), enhance safety (e.g. cardiac-specific promoters or micro RNA target sites), and if required enhance function (using additional genes involved in pacemaker function, described in detail in WO/2009/120082) when administered to a subject in vivo. Lentiviral vectors potentially induce life-long gene expression as a result of their integration into the host genome, and they elicit a minimal immune response and can therefore be repeatedly administered allowing for patient specific titration of the therapy.

Lentiviral constructs can be optimized using methods known in the art to allow for stable cardiac-specific or stem cell gene expression. Such constructs include LV-CMV-X; LV-EF1α-X; LV-PGK; LV-cTnT-X; LV-cTnI; LV-CAG-X; LV-short troponin I-X; LV-CMV-immediate-early enhancer-ANF-X; LV-CMV-immediate-early enhancer-MLC2v; LV-αMHC-X; LV-MLC2v-X.

Other viral vectors that can be used in embodiments of the invention include the single-stranded (or double-stranded) adeno-associated virus (AAV) that has been adapted as one of the most effective gene delivery vehicles. It shows persistent and high-level transduction in a variety of tissues in vivo and an outstanding safety profile in human patients. The rapid development in AAV technology is finally reaching the point of materializing gene therapy benefit in patients. In general, AAV vectors contain replication and control sequences compatible with the host cell are used. A suitable vector, such as a single AAV vector will typically carry viral inverted terminal repeats (ITR) at the ends, the promoters, and microgene and polyA.

AAV have been shown to have a broad host range (for pulmonary expression) and persists in muscle, therefore recombinant AAV (rAAV) and AAV-Biological pacemaker constructs that incorporate rAAV described herein may be employed to express one or more genes in any animal, and particularly in mammals, preferably humans and domestic animals and primates. Both human and veterinary uses are particularly preferred.

The gene being expressed is a DNA segment encoding the enumerated HCN or SkM1 channels, with whatever control elements (e.g., promoters, operators) are desired by the user.

Adeno-associated virus vectors have certain advantages over the adenovirus vectors. First, like adenovirus, AAV can efficiently infect non-dividing cells, such as the targeted cardiac cells. Second, all the AAV viral genes are eliminated in the vector. Since the viral-gene-expression-induced immune reaction is no longer a concern, AAV vectors are safer than adenovirus vectors. Third, wild type AAV is an integration virus by nature, and integration into the host chromosome will stably maintain its transgene in the cells. Fourth, recombinant AAV vectors used for the BP constructs described herein can persist as episomal molecules for years in mammalian tissues such as muscle, in particular in rodent, dogs and human. The episomal form is considered the predominant form for AAV vector mediated transduction in tissues in vivo. Fifth, AAV is an extremely stable virus, which is resistant to many detergents, pH changes and heat (stable at 56° C. for more than an hour). It can be lyophilized and redissolved without losing its activity. Therefore, it is a very useful delivery vehicle for gene therapy.

Adeno-associated viral (AAV) vectors are well established as safe in clinical trials. Adeno-associated virus of many serotypes, especially AAV2, have been extensively studied and characterized as gene therapy vectors. AAV serotypes for use in embodiments of the present invention include AAV1, AAV2, AAV3, AAV4, AAV5, AAV6, AAV7, AAV8, AAV9, AAV10, AAV11, or AAV12, or a hybrid serotype thereof. Certain preferred AAV are those that can accommodate the SkM1 gene, including but not limited to AAV2 and AAV8.

In the methods of the invention, AAV of any serotype can be used so long as the vector is capable of transducing cardiac cells. (See, e.g., Gao et al. [2002] PNAS 99:11854-11859; and Machida ed., Viral Vectors for Gene Therapy: Methods and Protocols, Humana Press, 2003). Other serotypes besides those listed herein can be used. Furthermore, pseudotyped AAV vectors may also be utilized in the methods described herein. Pseudotyped AAV vectors are those which contain the genome of one AAV serotype in the capsid of a second AAV serotype; for example, an AAV vector that contains the AAV2 capsid and the AAV1 genome or an AAV vector that contains the AAV5 capsid and the AAV 2 genome (Auricchio et al. [2001] Hum. Mol. Genet. 10(26):3075-81).

Those skilled in the art will be familiar with the preparation of functional AAV-based gene therapy vectors. Numerous references to various methods of AAV production, purification and preparation for administration to human subjects can be found in the extensive body of published literature (see, e.g., Viral Vectors for Gene Therapy: Methods and Protocols, ed. Machida, Humana Press, 2003). Additionally, AAV-based gene therapy targeted to cells of the CNS has been described in U.S. Pat. Nos. 6,180,613 and 6,503,888. In AV and AAV vectors, all viral genes can be removed and replaced with a therapeutic expression cassette for the HCN and sodium channel genes. Until recently because of inherent limitations of the viral capsid, the maximal vector genome that could be assembled in a single AAV virion was limited to typically less than about 4.5 kb, however, adeno-associated virus (AAV) can be adapted as a delivery vector/construct to carry a “cargo” capacity of 6 kb and it the most effective gene delivery vehicle to muscle cells. (43). Viruses carrying large genomes (up to 5.6 kb) have been reported to replicate and produce infectious virions, containing a completely recombinant genome encoding the chloramphenicol acetyltransferase (CAT) gene, ranging in size from 1,918 to 6,019 by (44, 45) An in vivo study by Sarkar et al. utilized AAV8 to package a 5.6-kb canine FVIII cDNA construct that was later found to give 100% correction of plasma FVIII activity in a haemophilia. (46). Thus AA2 and AA8 vectors can carry large genes the size of SkM1.

Other methods for transducing a cell with a viral vector have shown that synthetic introns trans-splicing methods wherein a large therapeutic gene is split into two arbitrarily designated “exons,” one carrying the 5′ expression cassette and the splicing donor and the other carrying the splicing acceptor and the 3′ expression cassette (47, and PCT/US2008/000717). Expression is achieved by ITR-mediated recombination and subsequent splicing of the recombinant genome in co-infected cells. The gene-splitting site is defined by the splicing signals in both intron and exons. Lai et al. (47) describes a method for intron optimization that augments gene expression from trans-splicing vectors in which the endogenous intron in the gene is replaced with an optimized synthetic intron to overcome the mRNA accumulation barrier at a poor junction. can effectively replace endogenous introns in trans-splicing vectors which helps overcome the mRNA accumulation barrier at a poor junction, thereby achieving a therapeutic level of protein expression in vivo. Certain embodiments include delivering the therapeutic HCN and sodium channel genes in the dual vector system. Other multiple AAV constructs used to express a single gene are described by Lostal et al. (42). Tri-AAV vectors were used to expand the total carrying capacity of the AAV vectors to 15 kb, which not permits delivery a large therapeutic gene like SkM1, but also provides more space for engineering various regulatory elements into the viral vector construct (expression cassette).

In certain methods of the invention, an AAV vector for use in embodiments of the invention comprises the gene for HCN2, HCN4, or SkM1 or a biologically active fragment or variant thereof. In various embodiments, the genes can be a human, mouse, or rat genes, but is not limited to these examples.

In various embodiments, the AAV vector is a single-stranded or double-stranded AAV. In various embodiments, the AAV vector is a self-complementary AAV (scAAV).

In various embodiments, the AV or AAV vector is a virus particle. In accordance with the present invention, the serotype of the virus particle can be one of AAV1, AAV2, AAV3, AAV4, AAV5, AAV6, AAV7, AAV8, AAV9, AAV10, AAV11, or AAV12, or a hybrid serotype thereof. Certain preferred AAV are those that can accommodate the SkM1 gene, including but not limited to AAV2 and AAV8.

The level of transgene expression in eukaryotic cells is largely determined by the transcriptional promoter within the transgene expression cassette. Promoters that show long-term activity and are tissue- and even cell-specific are used in some embodiments.

Typically, the vector is constructed so as to provide operatively linked components of control elements. For example, a typical vector includes a transcriptional initiation region, a nucleotide sequence of the protein to be expressed, and a transcriptional termination region. When a rAAV vector is used, such an operatively linked construct is typically flanked at its 5′ and 3′ regions with AAV ITR sequences, which are required viral cis elements. The control sequences can often be provided by promoters derived from viruses such as polyoma, Adenovirus 2, cytomegalovirus (CMV), and Simian Virus 40; or from the promoter regions of human genes such as promoter regions of the phosphoglycerate kinase (PGK) gene, the cardiac tropinin T (cTnT) gene, the myosin light chain 2v (MLC-2v) gene, and the brain natriuretic brain (BNP) gene, or combinations thereof such as the MLC-2v promoter fused to a minimal CMV enhancer. Non-limiting examples of promoters are described in the literature: the cytomegalovirus (CMV) promoter (Kaplitt et al. [1994] Nat. Genet., 8:148-154), CMV/human .beta.3-globin promoter (Mandel et al. [1998] J. Neurosci., 18:4271-4284), GFAP promoter (Xu et al. [2001] Gene Ther., 8:1323-1332), the 1.8-kb neuron-specific enolase (NSE) promoter (Klein et al. [1998] Exp. Neurol., 150:183-194), chicken beta actin (CBA) promoter (Miyazaki Gene, 79:269-277) and the .beta.-glucuronidase (GUSB) promoter (Shipley et al. [1991] Genetics, 10:1009-1018). Cardiac-specific expression is achieved with the use of cardiac-specific promoters. Examples of such promoters which have demonstrated specific and efficient expression in cardiac myocytes include the brain natriuretic brain (BNP) promoter, the cardiac tropinin T (cTnT) promoter, and the myosin light chain 2v (MLC-2v) promoter fused to a minimal cytomegalovirus (CMV) enhancer. Certain embodiments also include the CAG promoter (51).

Vectors may also contain cardiac enhancers to increase the expression of the transgene in the targeted regions of the cardiac conduction system. Such enhancer elements may include the cardiac specific enhancer elements derived from Csx/Nkx2.5 regulatory regions disclosed in the published U.S. Patent Application 20020022259, the teachings of which are herein incorporated by reference. The embodiment of the methods the pacemaker activity of the HCN channel is enhanced by co-expressing its beta subunit, MiRP1 either by including the gene for MiRP1 in the same viral vector construct or by further administering a different viral vector construct comprising a gene encoding a biologically active MiRP1 beta subunit.

For some gene therapy applications, it may be desirable to control transcriptional activity. To this end, pharmacological regulation of gene expression with BP constructs can been obtained by including various regulatory elements and drug-responsive promoters as described, for example, in Haberma et al. (1998) Gene Ther., 5:1604-16011; and Ye et al. (1995) Science, 283:88-91. One such regulator is doxorubicin.

In other embodiments different strategies can be used to increase HCN and Na channel availability at (slightly) depolarized potentials, such as introducing subunits of the channel, regulatory proteins that increase channel expression or introduction of mutations or microRNAs. Beta subunits that can be overexpressed to enhance sodium channel function include SCN1B, SCN2B, SCN3B and SCN4B and any of their mutant variants. Other genes that may be modified to increase sodium channel function include genes involved in their glycosylation and phosphorylation (e.g. Ca²⁺/calmodulin dependent kinase II), adaptor proteins that connect these channels to the cytoskeleton (e.g. ankyrins), and proteins that mediate their trafficking form the endoplasmatic reticulum to the sarcolemmal membrane (e.g. glycerol-3-phosphate dehydrogenase 1-like protein), and other proteins that have been shown to affect the biophysical properties of sodium channels such as fibroblast-like growth factors (e.g. FGF13UP/Q; (52). Alternatively, sodium channel function may be enhanced by the introduction of mutations that alter the biophysical properties of native Nav1.5 channels. Examples of such mutations include mutations that increase the peak sodium current (e.g. Y1795C) and mutations that shift the voltage dependence of inactivation towards more depolarized potentials. Mutations can be introduced into the native SCN5A gene using homologous recombination or comparable techniques. Additionally, sodium channel function may be enhanced using overexpression of small interference RNAs or removal of negatively regulating micro RNAs.

### Methods of Treatment

In various embodiments, the present invention provides a method of treating cardiac pacing or conduction dysfunction that is associated with sinoatrial node dysfunction and/or atrioventricular conduction block in a subject. Embodiments include providing a composition comprising viral or non-viral vector(s) as described herein that include HCN and sodium channel genes, such as HCN2 and SkM1, or a biologically active fragment or variant thereof; and administering a therapeutically effective amount of the composition to the heart of subject, preferably by intramyocardial/intramuscular injection, or by or infusion into the coronary vasculature hereby treating the disease-state in the subject. The therapeutic genes HCN2 and SkM1 can be in the same vector if the vector can accommodate the size, but the genes are typically in separate vectors that can be administered at the same time or in sequence. Treatment may consist of a single injection into the targeted area, or injections may be repeated in the targeted area.

In various embodiments, the therapeutically effective amount of the composition comprises about 1×10⁹-1×10¹⁴, 1×10⁹-1×10¹², 1×10¹⁰-1×10¹², 1×10¹²-1×10¹⁴, 1×10¹²-1×10¹⁵ or 1×10¹⁵-1×10¹⁸ genome copies (gc) of the rAAV vector per kg of body weight of the subject. In various embodiments, the composition is administrated to the subject more than once depending on the initial response and it can be repeated during the life time of the subject as deemed necessary and would be apparent to a person of skill in the art. See Brenner et al., 2014/0309288. Because injection into the heart is relatively non-invasive, repeated treatment is reasonable (1).

The terms “genome particles (gp),” or “genome equivalents,” as used in reference to a viral titre, refer to the number of virions containing the recombinant AAV DNA genome, regardless of infectivity or functionality. The number of genome particles in a particular vector preparation can be measured by procedures such as described in the Examples herein, or for example, in Clark et al. (1999) Hum. Gene Ther., 10:1031-1039; Veldwijk et al. (2002) Mol. Ther., 6:272-278.

The terms “infection unit (iu),” “infectious particle,” or “replication unit,” as used in reference to a viral titre, refer to the number of infectious and replication-competent recombinant AAV vector particles as measured by the infectious center assay, also known as replication center assay, as described, for example, in McLaughlin et al. (1988) J. Virol., 62:1963-1973

The term “transducing unit (tu)” as used in reference to a viral titre, refers to the number of infectious recombinant AAV vector particles that result in the production of a functional transgene product as measured in functional assays such as described in Examples herein, or for example, in Xiao et al. (1997) Exp. Neurobiol., 144:113-124; or in Fisher et al. (1996) J. Virol., 70:520-532 (LFU assay).

In some embodiments, the method of treating a disease comprises administration of a high titre vector such as an AAV vector carrying a therapeutic gene so that the gene product is expressed at a therapeutic level in transduced cardiac cells in the area of injection. In some embodiments, the viral titre of the composition is at least: (a) 5, 6, 7, 8, 8.4, 9, 9.3, 10, 15, 20, 25, or 50×10¹² gp/ml); (b) 5, 6, 7, 8, 8.4, 9, 9.3, 10, 15, 20, 25, or 50×10⁹ tu/ml); or (c) 5, 6, 7, 8, 8.4, 9, 9.3, 10, 15, 20, 25, or 50 (×10¹⁰ iu/ml). See Pasini et al., 2014/0356327.

In some embodiments, the viral titre of the composition chosen to achieve a therapeutic level at a distance of about 0-2, 3, 5, 8, 10, 15, 20, 25, 30, 35, 40 mm from the administration site. The total volume of material to be administered, and the total number of vector particles to be administered, will be determined by those skilled in the art based upon known aspects of gene therapy. Therapeutic effectiveness and safety can be tested in an appropriate animal model.

High titre AAV BP constructs can be produced using techniques known in the art, e.g., as described in U.S. Pat. No. 5,658,776 and Machida, ed., Viral Vectors for Gene Therapy: Methods and Protocols, Humana Press, 2003.

Genes and vectors of choice can be made by traditional PCR-based amplification and known cloning techniques. Alternatively, they can be made by automated procedures that are well known in the art. A gene encoding an HCN or sodium channel gene for use in embodiments of the invention should include a start codon to initiate transcription and a stop codon to terminate translation. Suitable HCN and Sodium channel genes for use with the invention can be obtained from a variety of public sources including, without limitation, GenBank (National Center for Biotechnology Information [NCBI]), EMBL data library, SWISS-PROT (University of Geneva, Switzerland), the PR-International database; and the American Type Culture Collection (ATCC)(10801 University Boulevard, Manassas, Va. 20110-2209). See generally, Benson, D. A. et al, Nucl. Acids. Res., 25:1 (1997) for a description of GenBank. The particular polynucleotides useful with the present invention are readily obtained by accessing public information from GenBank.

### Pharmaceutical Compositions

Pharmaceutical compositions that include a biological pacemaker AAV construct comprising an HCN2 or SkM1gene can be made simply dissolving an AAV vector in phosphate buffered saline (PBS) or in N-2-Hydroxyethylpiperazine-N′-2-ethanesulfonic acid (HEPES) buffered saline has been demonstrated to be sufficient to provide a vehicle useful for muscle tissue expression, and there are no known restrictions on the carriers or other components that can be coadministered with the vector (although compositions that degrade DNA should be avoided in the normal manner with vectors). In various embodiments, pharmaceutical compositions comprising the biological pacemaker vectors according to the invention may be formulated for delivery via injection to the heart.

For purposes of intramyocardial/intramuscular injection, solutions in an adjuvant such as sesame or peanut oil or in aqueous propylene glycol can be employed, as well as sterile aqueous solutions. Such aqueous solutions can be buffered, if desired, and the liquid diluent first rendered isotonic with saline or glucose. Solutions of the AAV vector as a free acid (DNA contains acidic phosphate groups) or a pharmacologically acceptable salt can be prepared in water suitably mixed with a surfactant such as hydroxypropylcellulose. A dispersion of AAV viral particles can also be prepared in glycerol, liquid polyethylene glycols and mixtures thereof and in oils. Under ordinary conditions of storage and use, these preparations contain a preservative to prevent the growth of microorganisms. In this connection, the sterile aqueous media employed are all readily obtainable by standard techniques well-known to those skilled in the art (PCT/US2008/000717).

The pharmaceutical forms suitable for injectable use include sterile aqueous solutions or dispersions and sterile powders for the extemporaneous preparation of sterile injectable solutions or dispersions. In all cases the form must be sterile and must be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, liquid polyethylene glycol and the like), suitable mixtures thereof, and vegetable oils. The proper fluidity can be maintained, for example, by the use of a coating such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal and the like. In many cases it will be preferable to include isotonic agents, for example, sugars or sodium chloride. Prolonged absorption of the injectable compositions can be brought about by use of agents delaying absorption, for example, aluminum monostearate and gelatin.

Sterile injectable solutions are prepared by incorporating the AAV vector in the required amount in the appropriate solvent with various of the other ingredients enumerated above, as required, followed by filtered sterilization. Generally, dispersions are prepared by incorporating the sterilized active ingredient into a sterile vehicle which contains the basic dispersion medium and the required other ingredients from those enumerated above. In the case of sterile powders for the preparation of sterile injectable solutions, the preferred methods of preparation are vacuum drying and the freeze drying technique, which yield a powder of the active ingredient plus any additional desired ingredient from the previously sterile-filtered solution thereof.

The therapeutic compounds described herein may be administered to a mammal alone or in combination with pharmaceutically acceptable carriers. The relative proportions of active ingredient and carrier are determined by the solubility and chemical nature of the compound, chosen route of administration and standard pharmaceutical practice. The dosage of the present therapeutic agents which will be most suitable for prophylaxis or treatment will vary with the form of administration, the particular compound chosen and the physiological characteristics of the particular patient under treatment. Generally, small dosages will be used initially and, if necessary, will be increased by small increments until the optimum effect under the circumstances is reached.

The BP constructs according to the invention may be delivered in a therapeutically effective amount. The precise therapeutically effective amount is that amount of a composition comprising the BP construct that will yield the most effective results in terms of efficacy of treatment in a given subject. This amount will vary depending upon a variety of factors, including but not limited to the characteristics of the therapeutic compound (including activity, pharmacokinetics, pharmacodynamics, and bioavailability), the physiological condition of the subject (including age, sex, disease type and stage, general physical condition, responsiveness to a given dosage, and type of medication), the nature of the pharmaceutically acceptable carrier or carriers in the formulation, and the route of administration. One skilled in the clinical and pharmacological arts will be able to determine a therapeutically effective amount through routine experimentation, for instance, by monitoring a subject's response to administration of a compound and adjusting the dosage accordingly. For additional guidance, see Remington: The Science and Practice of Pharmacy (Gennaro ed. 20th edition, Williams & Wilkins PA, USA) (2000).

It is also preferred to use a buffer in the composition to minimize pH changes in the solution before lyophilization or after reconstitution. Most any physiological buffer may be used including but not limited to citrate, phosphate, succinate, and glutamate buffers or mixtures thereof. In some embodiments, the concentration is from 0.01 to 0.3 molar. Surfactants that can be added to the formulation are shown in EP Nos. 270,799 and 268,110.

### Kits

Embodiments are directed to a kit comprising: a composition comprising one or more of the herein described biological pacemaker constructs comprising the therapeutic genes and a promoter, and instructions for using the composition for treating, inhibiting, preventing, reducing the severity of and/or reducing the progression of a disease-state in a subject. The constructs include viral and non-viral constructs that have the gene for HCN or for SkM1, or both genes in a single construct.

The materials or components assembled in the kit can be provided to the practitioner stored in any convenient and suitable ways that preserve their operability and utility. For example the components can be in dissolved, dehydrated, or lyophilized form; they can be provided at room, refrigerated or frozen temperatures. The components are typically contained in suitable packaging material(s). As employed herein, the phrase “packaging material” refers to one or more physical structures used to house the contents of the kit, such as inventive compositions and the like. The packaging material is constructed by well-known methods, preferably to provide a sterile, contaminant-free environment. As used herein, the term “package” refers to a suitable solid matrix or material such as glass, plastic, paper, foil, and the like, capable of holding the individual kit components. Thus, for example, a package can be a glass vial used to contain suitable quantities of a composition containing a volume of the AAV1-P0-ICE vector. The packaging material generally has an external label which indicates the contents and/or purpose of the kit and/or its components.

### Protein Variants

The HCN and sodium channels of the present invention include biologically-active protein variants with substantial homology to the endogenous protein, or an identified mutant or chimera thereof or biologically active fragment thereof. For the purpose of this invention, genes encoding variants of a protein include substantially homologous proteins that are naturally occurring and retain the desired biological activity. As used herein, two proteins (or a region of the proteins) are substantially homologous when the amino acid sequences are at least 70-75%, typically at least 80-85%, and most typically at least 90-95%, 97%, 98% or 99% or more homologous. A substantially homologous amino acid sequences will be encoded by a nucleic acid sequence hybridizing to the corresponding nucleic acid sequence, or portion thereof, under stringent conditions. Variants of the gene include making conservative amino acid substitutions such as Aromatic Phenylalanine Tryptophan Tyrosine Hydrophobic Leucine Isoleucine Valine Polar Glutamine Asparagine Basic Arginine Lysine Histidine Acidic Aspartic Acid Glutamic Acid Small Alanine Serine Threonine Methionine Glycine.

A variant polypeptide can differ in amino acid sequence by one or more substitutions, deletions, insertions, inversions, fusions, and truncations or a combination of any of these. Fully functional biologically active variants/typically contain only conservative variations or variations in non-critical residues or in non-critical regions. Biologically active variants can also contain substitution of similar amino acids, which results in no change or an insignificant change in function. Substantial homology can be to the entire mRNA encoding the endogenous protein of interest or to mRNA encoding a biologically-active fragment or variant thereof. Accordingly, a fragment can comprise any length that retains one or more of the biological activities of the protein. Fragments can be discrete (not fused to other amino acids or polypeptides) or can be within a larger polypeptide.

### EXAMPLES

### Example 1

### Overexpression of SkM1 Enhances HCN2-Based Biological Pacemaker Function

Experiments were performed with the use of protocols approved by the Columbia University Institutional Animal Care and Use Committee and conform to the Guide for the Care and Use of Laboratory Animals (National Institutes of Health publication No. 85-23, revised 1996).

### Materials & Methods

### Adenoviral Constructs:

Adenoviral constructs of green fluorescent protein (GFP), mouse HCN2 and rat SkM1, all driven by the CMV promoter, were prepared as described previously. Qu J, Plotnikov A N, Danilo P, et al. Expression and function of a biological pacemaker in canine heart. Circulation 2003; 107:1106-1109; Lau D H, Clausen C, Sosunov E A, et al. Epicardial border zone overexpression of skeletal muscle sodium channel SkM1 normalizes activation, preserves conduction, and suppresses ventricular arrhythmia: an in silico, in vivo, in vitro study. Circulation 2009; 119:19-27. We prepared an empty adenoviral vector as an additional control vector. For consistency with earlier studies (3) we prepared 3×10¹⁰ fluorescence focus forming units of one vector and mixed this with an equal amount of the other vector in a total volume of 700 μL. We did not use a single vector for delivery because the size of SkM1 (5.5 kb) was too large to combine in a single adenoviral vector at the time the experiments were done. There are now standard procedures for making AV vectors and AAV vectors that can accommodate up to 6 kb.

AAV vectors can be purified, for example, by iodixanol gradient centrifugation followed by column chromatography with HiTrap Q anion-exchange columns (GE Healthcare, Piscataway, N.J.). The virus-containing fractions can be concentrated with Centricon 100-kDa molecular weight cutoff (MWCO) centrifugal devices (Millipore, Billerica, Mass.) and the titer (genome copies [GC]/ml) can be determined by real-time PCR amplification with primers and probe specific for the bovine growth hormone polyadenylation signal. Brenner et al, 2014/0309288.

### Animal Monitoring Procedures:

ECG, 24-hour Holter monitoring, pacemaker log record check, and overdrive pacing were performed daily for 7-8 days. For each dog, the percent of electronically induced beats was calculated daily. Endogenous pacemaker activity was suppressed by 30 seconds of electronic pacing at 80 bpm or ˜10% above intrinsic rhythm.

Twenty-four hour monitoring was performed via Holter ECG (Rozinn, Scottcare, Glendale, N.Y.). We calculated maximal beating rates daily from 30-sec strips of a stable rhythm at maximal rate. We performed detailed analysis of the percentages of matching and non-matching beats (using pace-mapped beats at time of implant as a reference), bigeminal, and electronically paced beats, 24 hr average beating rate of matching rhythms, and HRV on Holter ECG recordings registered during steady-state gene expression (days 5-7; one day per animal). To analyze circadian variation, we compared the rate of pace-mapped beats and dependence on electronic back-up pacing during sleep (2-4 AM) versus during feeding and physical activity (8-10 AM). In the analysis of HRV we calculated the standard deviation (SD) of all pace-mapped beats to assess their instantaneous RR-interval variability (SD1) and the SD of long-term continuous RR-interval variability (SD2).

### Intact Canine Studies:

Adult mongrel dogs were prepared, anesthetized, and fitted with electronic pacemakers (VVI 35 beats/min) and underwent radiofrequency ablation of the atrioventricular node as described previously (2). One series of animals was injected in the left bundle branch (LBB) with the appropriate adenovirus construct to obtain the following groups: HCN2 (n_12), including 7 previously reported HCN2/green fluorescent protein (GFP)-treated animals (8), 3 current HCN2/GFP-treated animals, and 2 animals injected with HCN2 plus empty vector; SkM1/GFP (designated SkM1; n_6); and HCN2/SkM1 (n_6). Outcomes in the HCN2/empty group were comparable to those with HCN2/GFP and were therefore combined into one group designated HCN2.

Left thoracotomies were performed on a second series of animals using previously described methods (6), and adenovirus constructs were injected into 3 left ventricular (LV) anterobasal epicardial sites to obtain the following groups: HCN2/GFP (n_10; designated HCN2), SkM1/GFP (n_7; designated SkM1), and HCN2/SkM1 (n_6). Injections were in close proximity (approximately 4 mm) to one another. The injection site was marked with 2 sutures.

### Immunohistochemistry:

HCN2 and SkM1 overexpression were validated by immunohistochemistry. Tissue blocks were snap-frozen in liquid nitrogen, and 5 um serial sections were cut with a cryostat (Microm HM505E) and air dried. Sections were washed in PBS, blocked for 20 minutes with 10% goat serum, and incubated overnight at 4° C. with anti-SkM1 antibody (1:200, Sigma-Aldrich, St Louis, Mo.) and anti-HCN2 antibody (1:200, Alomone, Jerusalem, Israel). Antibody bound to target antigen was detected by incubating sections for 2 hours with goat anti-mouse IgG labelled with Cy3 (red fluorescence for SkM1) and goat anti-rabbit IgG labelled with Alexa 488 (green fluorescence for HCN2), images were collected with a Nikon E800 fluorescence microscope.

### Statistical Analysis:

Data are presented as mean±SEM in cases where data follow a normal Gaussian distribution. Statistical significance was examined by t-test or by two-way ANOVA followed by Bonferroni's post-hoc test. In the following datasets we did not detect a normal Gaussian distribution: % paced (daily pacemaker logs), % non-matching rhythm (Holter), % bigeminy (Holter), % paced (Holter), % paced (morning; pacemaker log). In these cases, data are presented as median and interquartile range. Statistical significance was examined by Kruskal-Wallis one-way ANOVA followed by Dunn's multiple comparison test and Mann Whitney test or Wilcoxon matched-pairs signed rank test. P<0.05 was considered significant.

### Beta-Adrenergic Responsiveness:

To evaluate β-adrenergic responsiveness at termination of the study, epinephrine (1.0 μg/kg/min) was infused for 10 minutes as previously reported (Bucchi A, Plotnikov, AN, Shlapakova I, et al. Wild-type and mutant HCN channels in a tandem biological-electronic cardiac pacemaker. Circulation 2006; 114:992-999.) and maximum rate response of the pace-mapped rhythm was recorded.

### Tissue Bath Studies on LBB Preparations:

Preparations were placed in a 4-mL chamber perfused with Tyrode's solution (37° C., pH 7.3 to 7.4) at a rate of 12 mL/min and were permitted to beat spontaneously. Tyrode's solution containing isoproterenol (0.001-0.1 μM) followed by isoproterenol plus TTX (0.1 μM) were applied respectively. Transmembrane AP signals were acquired as described previously. (4,5)

### Tissue Bath Studies on Subepicardial Myocardial Bundles:

Seven days after subepicardial adenovirus injection, subepicardial myocardial fascicles (˜0.5 mm in diameter, 6-10 mm long) were isolated from the injection sites and from remote sites, 3-4 cm from the injected region. Preparations were mounted in a 2-compartment tissue bath, (25, 26) whose compartments were separated by a plastic partition having an opening 0.3-0.6 mm in diameter, permitting each preparation to slide through and fit snugly. Each compartment was independently perfused with Tyrode's solution.

Preparations were driven at a cycle length of 1 sec by current pulses delivered through Ag—AgCl electrodes placed in each compartment. Every 10th regular stimulus was replaced by a 30-ms depolarizing current pulse whose amplitude was gradually increased from subthreshold to threshold levels.

Conventional microelectrodes were used to record transmembrane potentials at locations within 0.1-0.2 mm of the partition. Phase 0 upstroke velocity was measured by analog differentiation of the transmembrane potential.

### Example 2

### Results of Intact Animal Studies

### Baseline Function

Biological pacing effectiveness was evaluated in light of baseline heart rates, escape times after overdrive pacing, and percentage time during which the backup electronic pacemaker drove the heart (FIG. 1). These parameters were compared in animals injected with biological pacemakers into the LBB or LV subepicardium. Electrocardiograms (ECGs) were recorded while animals rested quietly on a table (baseline beating rates). Over 7 days, biological pacemaker function in HCN2/SkM1 LBB-injected animals was superior (i.e., faster basal rates, shorter escape times, and lower percentage of electronically stimulated beats) to that of animals with HCN2 or SkM1 alone, and was superior to that of animals with LV subepicardial injection of HCN2/SkM1.

### Autonomic Modulation

Sensitivity to autonomic modulation of pace-mapped rhythms was studied via 24-h ECG recordings. Faster beating rates were reached in HCN2/SkM1 LBB-injected animals than those injected with HCN2 or SkM1 (FIG. 2A). At 5 to 7 days, beating rates were significantly faster in animals that received HCN2/SkM1 into the LBB as compared with subepicardial injection (FIG. 2B). Typical recordings of maximal beating rates in LBB-injected animals are in FIGS. 2C, 2D, and 2E. A detailed analysis of percentage pace-mapped rhythms and their autonomic modulation was performed on the ECG Holter recordings at 5 to 7 days. The percentage of matching pace-mapped beats was significantly higher in HCN2/SkM1 LBB-injected animals (95% of all beats), requiring less pacemaker backup than the respective HCN2- and SkM1-injected groups (p less than 0.05) (FIG. 3A). The percentage of matching beats in animals that received HCN2/SkM1 into subepicardium was lower (approximately 60%) and did not differ from that of HCN2 and SkM1 control groups. Animals injected with SkM1 alone either into the subepicardium or LBB showed persistent bigeminy or trigeminy in more than 10% of beats, whereas no such arrhythmias were detected in animals injected with HCN2 or HCN2/SkM1 (p less than 0.05) (FIG. 3A). The percentage of electronically paced beats was reduced in the HCN2/SkM1-LBB group to 0% of all beats (p less than 0.05 vs. respective HCN2 and SkM1 groups) (FIG. 3A). The 24-h average rate of pace-mapped rhythms is summarized in FIG. 3B, showing a faster rate in HCN2/SkM1-LBB versus the HCN2-LBB and SkM1-LBB groups (p less than 0.05). These results are consistent with the 5- to 7-day averages of baseline and maximal beating rates reported in FIG. 2. Finally, animals that received HCN2 into the LBB exhibited faster 24-h average beating rates than animals that received HCN2 into the subepicardium (p less than 0.05) (FIG. 3B). To test whether the changes in beating rate and dependence on backup electronic pacing were consistent with what would be expected based on a normal circadian rhythm, we compared these parameters during 2 h of sleep (2:00 to 4:00 AM) with 2 h of feeding and activity (8:00 to 10:00 AM). Regardless of injection site, HCN2 and HCN2/SkM1 groups exhibited a significant rate acceleration of pace-mapped rhythms from morning to night (p less than 0.05) (FIG. 3C). During sleep as well as during feeding and activity, pace-mapped rhythms were significantly faster in HCN2/SkM1 LBB-injected animals as compared with those in HCN2-LBB and SkM1-LBB groups (p less than 0.05) (FIG. 3C).

Both HCN2-LBB and HCN2/SkM1-LBB groups exhibited faster beating rates in the morning than the respective subepicardially injected groups (p less than 0.05) (FIG. 3C). The percentage of electronically paced beats during night and morning is summarized in FIG. 3D. Subepicardially or LBB-injected animals that received HCN2 exhibited a lower percentage of electronic pacing in the morning than at night (p less than 0.05) (FIG. 3D).

Poincaré plots of pace-mapped rhythms also demonstrated differences in autonomic modulation as analyzed by heart rate variability (HRV) among animals with the 3 gene constructs injected into the LBB (FIG. 4A). Quantitative analysis of SD parameters revealed that the level of parasympathetic modulation expressed by short-term variation of heart rates (SD1) was comparable among the 3 groups tested (FIG. 5B, left panel). Sympathetic modulation, expressed as long-term variation of heart rates (SD2), was significantly reduced (i.e., normalized) in the HCN2/SkM1-LBB group as compared with that of animals LBB-injected with HCN2 (p less than 0.05) (FIG. 4B, middle panel). The parasympathetic-sympathetic balance (SD1:SD2 ratio) did not differ among the 3 groups (p less than 0.05) (FIG. 5B, right panel). Among the subepicardially injected groups, no significant changes in SD1, SD2, and SD1/SD2 were found.

On the final study day, all animals showed a significant rate acceleration upon intravenous epinephrine administration(1.0 g/kg/min; p less than 0.05) (FIG. 4C). Furthermore, during epinephrine infusion, animals subepicardially injected with HCN2/SkM1 exhibited faster beating rates than the respective HCN2 group (p less than 0.05). Similarly, during baseline and during epinephrine infusion, HCN2/SkM1 LBB injected animals showed significantly faster beating rates than their respective HCN2 or SkM1 groups (p less than 0.05). Finally, in HCN2/SkM1 LBB-injected animals, beating rates in baseline and epinephrine groups were significantly faster than in subepicardially injected animals (p less than 0.05).

### Isolated Tissue Studies

Representative examples and summary data from isolated tissue experiments conducted on LBB from HCN2-, SkM1- and HCN2/SkM1-injected animals are shown in FIGS. 5A and 5B. In normal Tyrode solution, beating rates did not differ among groups. However, when isoproterenol was added, HCN2/SkM1-treated preparations beat faster than the others (p less than 0.05). With isoproterenol 0.1 μM superfusion maintained, we added tetrodotoxin 0.1 μM, which selectively blocks SkM1 current (7). Tetrodotoxin significantly slowed the HCN2/SkM1-injected preparations, bringing their beating rates into the same range as the HCN2-injected preparations (FIG. 5B). This is consistent with a major contribution of SkM1 to the beating rates in the presence of isoproterenol. During superfusion with isoproterenol 0.1 μM, maximum diastolic potential was significantly more depolarized in HCN2-overexpressing tissue than in tissue that did not overexpress HCN2 (p less than 0.05) (FIG. 5B).

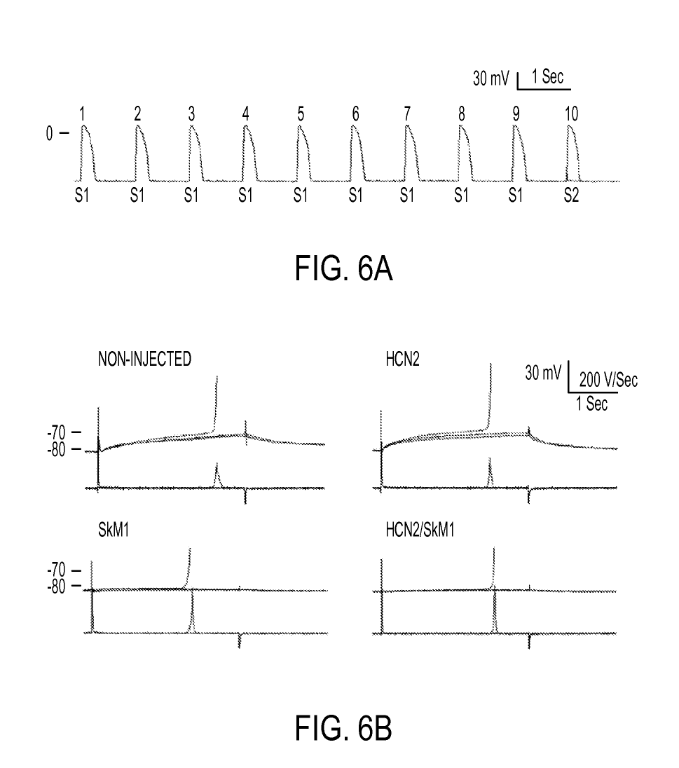

To test whether threshold potential shifts negatively in the presence of SkM1, we conducted experiments on dogs in which viral constructs were injected into myocardium. FIGS. 6A and 6B provide typical tracings and summary data. Data acquired from the first 9 action potentials (APs) per cycle that were stimulated normally confirmed the functional presence of SkM1 in the SkM1 and HCN2/SkM1 groups (data not shown). Specifically, as in previous reports (6, 8), SkM1 overexpression induced an increase in maximal upstroke velocity in the SkM1 and HCN2/SkM1 groups compared with those in the respective noninjected controls and the HCN2-injected group (p less than 0.05). The 10th AP was generated with a current pulse that was varied to identify the threshold potential for AP initiation. Threshold was reached at more negative voltages in SkM1- and HCN2/SkM1-injected preparations than in noninjected and HCN2-injected controls (p less than 0.05).

### Tissue Bath and Immunohistochemistry Studies:

To confirm the contribution of both HCN2 and SkM1 currents to the automaticity seen in HCN2/SkM1 injected animals we performed tissue bath experiments. FIG. 2 demonstrates a typical experiment in which we superfused endocardial tissue slabs (incorporating the construct injection site) with isoproterenol, 0.1 uM plus TTX, and after the TTX washout, with ivabradine. The reductions in rate seen after the application of TTX and ivabradine indicate contribution of respectively SkM1 and HCN2 to the basal, isoproterenol-enhanced rhythms. The presence of HCN2 and SkM1 proteins was subsequently confirmed immunohistochemically (FIG. 3).

### Autonomic Modulation of Biological Pacemaker Function:

Autonomic modulation of pacing rates is a potential key advantage of biological over electronic pacing (8). The extent of autonomic modulation that may be obtained using a biological approach likely depends on the gene construct. The results show that the average baseline beating rate in the HCN2/SkM1-LBB group was relatively rapid (approximately 80 beats/min) (FIG. 1), and the animals maintained robust rate acceleration, reaching average maximal rates of approximately 130 beats/min (FIG. 2). Furthermore, maximal beating rates remained within the physiological range, not exceeding 180 beats/min. This outcome is (1) superior to the slower maximal beating rates reported here for HCN2 or SkM1 alone (FIG. 2) and elsewhere for AC1 (4) in LBB-injected animals, (2) superior to results with injection of HCN2/SkM1 into subepicardium (FIG. 2), and (3) superior to the very rapid maximal rates reported for animals in which the chimera HCN212 and the combination of HCN2/AC1 were both injected into LBB (3,4).

The average beating rates was investigated by comparing a period of rest (2:00 to 4:00 AM) with one of physical activity and feeding (8:00 to 10:00 AM). Beating rates were in accordance with those expected with a normal response to circadian modulation (FIGS. 3C and 3D). The circadian response in the HCN2/SkM1-LBB group was also superior to that in animals with LBB gene transfer of HCN2 or SkM1 only and myocardial gene transfer of HCN2/SkM1.

Finally, sensitivity to parasympathetic and sympathetic modulation was investigated using analysis of HRV and infusion of epinephrine. The significant reduction in SD2 in the comparison of HCN2/SkM1-LBB with HCN2-LBB(FIG. 4B, middle panel) is unlikely an indication of reduced sensitivity to sympathetic modulation in the former given the strong in vitro (FIG. 5) and in vivo (FIG. 4C) responses to isoproterenol and epinephrine, respectively, which indicated greater sensitivity to sympathetic stimuli in HCN2/SkM1-LBB than HCN2-LBB preparations. In the HCN2-LBB group, accelerations (likely induced by sympathetic stimuli) and decelerations (likely resulting from reduced biological pacemaker function) were frequently observed at rest, when beating rates in the HCN2/SkM1 group were relatively stable. Therefore, it appears likely that sympathetic stimulation during rest in the HCN2/SkM1-LBB group was below the level of that in the HCN2-LBB group, although the 24-h average beating rates in the HCN2/SkM1-LBBgroup were higher (FIG. 3B). These data indicated that LBB-injected animals that received HCN2 likely manifested increased sympathetic activity during rest as a result of their slower beating rates. The lower average values found for SD2 in the HCN2/SkM1-LBB group therefore indicated reduced activity of the sympathetic system during rest rather than reduced sensitivity to sympathetic modulation (13). Mechanisms underlying pacemaker function based on HCN2/SkM1 gene transfer.

The microelectrode experiments on myocardial bundles obtained from subepicardially injected animals demonstrated the effect of SkM1 to move the threshold potential to more negative voltages (FIG. 6). This observation is significant because shifting the threshold in this direction would result in AP initiation earlier during phase 4 depolarization of automatic fibers. Although this change in AP threshold likely is a major mechanism by which SkM1 improves pacemaker function.

We previously reported the use of MSCs for the delivery of HCN2 current to myocardium and fabrication of a cell-based biological pacemaker that functioned stably over 6 weeks (20). In a different study, we also showed that the SkM1 current can be efficiently delivered to myocardium via the MSC platform (21). In the future, the MSC platform may offer an alternative means of gene delivery. However, MSCs show a tendency to migrate from the injection site, causing a loss of pacemaker function over time.

### REFERENCES

1. Rosen M R, Robinson R B, Brink P R, Cohen I S. The road to biological pacing. Nat Rev Cardiol 2011; 8:656-66.
2. Bucchi A, Plotnikov A N, Shlapakova I, et al. Wild-type and mutant HCN channels in a tandem biological-electronic cardiac pacemaker. Circulation 2006; 114:992-9.
3. Plotnikov A N, Bucchi A, Shlapakova I, et al. HCN212-channel biological pacemakers manifesting ventricular tachyarrhythmias are responsive to treatment with If blockade. Heart Rhythm 2008; 5:282-8.
4. Boink G J J, Nearing B D, Shlapakova I N, et al. Ca2+-stimulated adenylyl cyclase AC1 generates efficient biological pacing as single gene therapy and in combination with HCN2. Circulation 2012; 126:528-36.
5. Cingolani E, Yee K, Shehata M, Chugh S S, Marba'n E, Cho H C. Biological pacemaker created by percutaneous gene delivery via venous catheters in a porcine model of complete heart block. Heart Rhythm 2012; 9:1310-8.
6. Lau D H, Clausen C, Sosunov E A, et al. Epicardial border zone overexpression of skeletal muscle sodium channel SkM1 normalizes activation, preserves conduction, and suppresses ventricular arrhyth-mia: an in silico, in vivo, in vitro study. Circulation 2009; 119:19-27.
7. Protas L, Dun, W, Jia Z, et al. Expression of skeletal but not cardiac Na+ channel isoform preserves normal conduction in a depolarized cardiac syncytium. Cardiovasc Res 2009; 81:528-35.
8. Shlapakova I N, Nearing B D, Lau D H, et al. Biological pacemakers in canines exhibit positive chronotropic response to emotional arousal. Heart Rhythm 2010; 7:1835-40.
9. Miake J, Marba'n E, Nuss H B. Functional role of inward rectifier current in heart probed by Kir2.1 overexpression and dominant-negative suppression. J Clin Invest 2003; 111:1529-36.
10. Mattick P, Parrington J, Odia E, Simpson A, Collins T, Terrar D. Ca2+-stimulated adenylyl cyclase isoform AC1 is preferentially ex-pressed in guinea-pig sino-atrial node cells and modulates the If pacemaker current. J Physiol 2007; 582:1195-203.
11. Younes A, Lyashkov A E, Graham D, et al. Ca2+-stimulated basal adenylyl cyclase activity localization in membrane lipid microdomains of cardiac sinoatrial nodal pacemaker cells. J Biol Chem 2008; 283:14461-8.
12. Fagan K A, Mahey R, Cooper D M. Functional co-localization of transfected Ca2+-stimulable adenylyl cyclases with capacitative Ca2+ entry sites. J Biol Chem 1996; 271:12438-44.
13. Huikuri H V, Seppanen T, Koistinen, M J, et al. Abnormalities in beat-to-beat dynamics of heart rate before the spontaneous onset of life-threatening ventricular tachyarrhythmias in patients with prior myocardial infarction. Circulation 1996; 93:1836-44.
14. Dobrzynski H, Boyett M R, Anderson R H. New insights into pace-maker activity: promoting understanding of sick sinus syndrome. Circulation 2007; 115:1921-32.
15. Verkerk A O, Wilders R, van Borren M M, Tan H L. Is sodium current present in human sinoatrial node cells? Int J Biol Sci 2009; 5:201-4.
16. Zeng J, Rudy Y. Early afterdepolarizations in cardiac myocytes: mechanism and rate dependence. Biophys J 1995; 68:949-64.
17. Rudy Y. Reentry: insights from theoretical simulations in a fixed pathway. J Cardiovasc Electrophysiol 1995; 6:294-312.
18. Niwano K, Arai M, Koitabashi N, et al. Lentiviral vector-mediated SERCA2 gene transfer protects against heart failure and left ventricular remodeling after myocardial infarction in rats. Mol Ther 2008; 16:1026-32.
19. Li J, Sun W, Wang B, Xiao X, Liu X Q. Protein trans-splicing as a means for viral vector-mediated in vivo gene therapy. Hum Gene Ther 2008; 19:958-64.
20. Plotnikov A N, Shlapakova I, Szabolcs M J, et al. Xenografted adult human mesenchymal stem cells provide a platform for sustained biological pacemaker function in canine heart. Circulation 2007; 116:706-13.
21. Boink G J J, Lu J, Driessen H E, et al. Effect of SkM1 sodium channels delivered via a cell platform on cardiac conduction and arrhythmia induction. Circ Arrhythm Electrophysiol 2012; 5:831-40.
22. Boink, et al., HCN2/SkM1 Gene Transfer Into Canine Left Bundle Branch Induces Stable, Autonomically Responsive Biological Pacing at Physiological Heart Rates, Journal of the American College of Cardiology, Vol. 61, No. 11, 2013.
23. W. Catterall, et al., Pharmacol Rev 57:397-409, 2005.
24. G. Boink et al., Gene Therapy for Restoring Heart Rhythm, J Cardiovasc Pharmacol Ther 2014 19: 426.
25. Lau D H, et al. Epicardial border zone overexpression of skeletal muscle sodium channel SkM1 normalizes activation, preserves conduction, and suppresses ventricular arrhythmia: an in silico, in vivo, in vitro study. Circulation 2009 Jan. 6; 119(1):19-27.
26. Qu J, et al, HCN2 overexpression in new born and adult ventricular myocytes: distinct effects on gating and excitability. Circ Res 2001 Jul. 6; 89(1):E8-14.
27. Kass-Eisler A, Falck-Pedersen E, Alvira M, et al. Quantitative determination of adenovirus-mediated gene delivery to rat cardiac myocytes in vitro and in vivo. Proc Natl Acad Sci USA. 1993; 90(24):11498-11502.
28. Tripathy S K, Black H B, Goldwasser E, Leiden J M. Immune responses to transgene-encoded proteins limit the stability of gene expression after injection of replication-defective adenovirus vectors. Nat Med. 1996; 2(5):545-550.
29. Vassalli G, Bueler H, Dudler J, von Segesser L K, Kappenberger L. Adeno-associated virus (AAV) vectors achieve prolonged transgene expression in mouse myocardium and arteries in vivo: a comparative study with adenovirus vectors. Int J Cardiol. 2003; 90(2-3):229-238.
30. Zsebo K, Yaroshinsky A, Rudy J J, et al. Long-term effects of AAV1/SERCA2a gene transfer in patients with severe heart failure: analysis of recurrent cardiovascular events and mortality. Circ Res. 2014; 114(1):101-108.
31. Mingozzi F, Anguela X M, Pavani G, et al. Overcoming preexisting humoral immunity to AAV using capsid decoys. Sci Transl Med. 2013; 5(194):194ra92.
32. Jessup M, Greenberg B, Mancini D, et al. Calcium Upregulation by Percutaneous Administration of Gene Therapy in Cardiac Disease (CUPID): a phase 2 trial of intracoronary gene therapy of sarcoplasmic reticulum Ca21)-ATPase in patients with advanced heart failure. Circulation. 2011; 124(3):304-313.
33. Duan D, Yue Y, Engelhardt J F. Expanding AAV packaging capacity with trans-splicing or overlapping vectors: a quantitative comparison. Mol Ther. 2001; 4(4):383-391.
34. Bartel M A, Weinstein J R, Schaffer D V. Directed evolution of novel adeno-associated viruses for therapeutic gene delivery. Gene Ther. 2012; 19(6):694-700.
35. Naldini L, Blomer U, Gallay P, et al. In vivo gene delivery and stable transduction of nondividing cells by a lentiviral vector. Science. 1996; 272(5259):263-267.
36. Cingolani E, Ramirez Correa G A, Kizana E, et al. Gene therapy to inhibit the calcium channel beta subunit: physiological consequences and pathophysiological effects in models of cardiac hypertrophy. Circ Res. 2007; 101(2):166-175.
37. Niwano K, Arai M, Koitabashi N, et al. Lentiviral vector mediated SERCA2 gene transfer protects against heart failure and left ventricular remodeling after myocardial infarction in rats. Mol Ther. 2008; 16(6):1026-1032.
38. Naldini L. Ex vivo gene transfer and correction for cell-based therapies. Nat Rev Genet. 2011; 12(5):301-315.
39. Campochiaro P A. Gene transfer for ocular neovascularization and macular edema. Gene Ther. 2012; 19(2):121-126.
40. Guy H M, McCloskey L, Lye G J, Mitrophanous K A, Mukhopadhyay T K. Characterization of lentiviral vector production using microwell suspension cultures of HEK293T-derived producer cells. Hum Gene Ther Methods. 2013; 24(2):125-139.
41. Stewart H J, Leroux-Carlucci M A, Sion C J, Mitrophanous K A, Radcliffe P A. Development of inducible EIAV-based lentiviral vector packaging and producer cell lines. Gene Ther. 2009; 6(6):805-814.
42. Lostal W¹, Kodippili K, Yue Y, Duan D.; Full-length dystrophin reconstitution with adeno-associated viral vectors, Hum Gene Ther., 2014 June; 25(6):552-62.
43. Duan, D. Et al., From the Smallest Virus to the Biggest Gene: Marching Towards Gene Therapy for Duchenne Muscular Dystrophy, Published on Jul. 27, 2009 in Discovery Medicine.
44. Dong, J. Y., P. D. Fan, and R. A. Frizzell. 1996, Quantitative analysis of the packaging capacity of recombinant adeno-associated virus. Hum. Gene Ther. 7:2101-2112.
45. Grieger, J. JOURNAL OF VIROLOGY, August 2005, p. 9933-9944.
46. Sarkar, R et al. 2004. Total correction of hemophilia A mice with canine FVIII using an AAV 8 serotype. Blood 103:1253-1260).
47. Lai, Y., et al., Synthetic Intron Improves Transduction Efficiency of Trans-Splicing Adeno-Associated Viral Vectors, Hum Gene Ther. 2006; 17(10): 1036-1042.
48. U.S. patent application Ser. No. 11/490,760 Rosen et al., titled Biological bypass bridge with sodium channels, calcium channels and/or potassium channels to compensate for conduction block in the heart, Publication No. 20070053886.
49. WO/2009/120082, Boink et al, Means and methods for influencing electrical activity of cells.
50. Xue T, Lau C P, et al. Bioartificial sinus node constructed via in vivo gene transfer of an engineered pacemaker HCN channel reduces the dependence on electronic pacemaker in a sick-sinus syndrome model. Circulation 2006; 114:1000-1011.
51. Shunsuke Kawamoto, Qun Shi, Yoshio Nitta, Jun-ichi Miyazaki and Margaret D. Allen, Molecular Therapy (2005) 11, 980-985; doi: 10.1016/j.ymthe.2005.02.009 Widespread and early myocardial gene expression by adeno-associated virus vector type 6 with a bold beta-actin hybrid promoter.
52. Wang C et al. J Biol Chem. 2011; 286:24253-63.
53. Gao et al. PNAS 2002; 99:11854-11859.
54. Machida ed., Viral Vectors for Gene Therapy: Methods and Protocols, Humana Press, 2003.
55. Auricchio et al. Hum. Mol. Genet., 2001; 10(26):3075-81.

1-33. (canceled)

34. A pharmaceutical composition comprising an adenoviral vector construct, adeno-associated viral (AAV) vector construct, or retroviral vector construct, wherein each construct comprises either (a) a gene encoding a biologically active hyperpolarization-activated cyclic nucleotide-gated channel (HCN) or a biologically active fragment or variant thereof, or (b) a gene encoding a biologically active voltage gated SkM1 sodium channel or a biologically active fragment or variant thereof, or (c) both the HCN and SkM1 genes, and a pharmaceutically acceptable carrier.

35. The pharmaceutical composition of claim 34, wherein the HCN gene is selected from the group consisting of mouse HCN2 or human HCN2, and an isoform selected from the group consisting of mouse HCN1, human HCN1, mouse HCN4 and human HCN4 or biologically active fragments or variants thereof, and the sodium channel gene is rat SkM1 or human SkM1.

36. The pharmaceutical composition of claim 34, wherein the SkM1 gene encodes a neuronal isoform Nav 1.1, 1.2, 1.3, 1.6, 1.7 and 1.9 of the sodium channel gene.

37. The pharmaceutical composition of claim 34, wherein the gene encoding the hyperpolarization-activated cyclic nucleotide-gated HCN channel is selected from the group consisting of HCN2, HCN1 and HCN4 channel genes that encode a protein that is at least 95% identical to human HCN2, HCN1 and HCN4 or mouse HCN2, HCN1 and HCN4 channel, and the gene encoding the SkM1 channel encodes a protein that is at least 95% identical to human SkM1 or rat SkM1 protein.

38. The pharmaceutical composition of claim 34, wherein the composition comprises a retroviral vector selected from the group consisting of lentiviral vector (LV)-CMV-X; LV-EF1α-X; LV-PGK; LV-cTnT-X; LV-cTnI; LV-CAG-X; LV-short troponin I-X; LV-CMV-immediate-early enhancer-ANF-X; LV-CMV-immediate-early enhancer-MLC2v; LV-αMHC-X; LV-MLC2v-X, Herpes simplex, vaccine viruses and Gemlike Forest virus.

39. The pharmaceutical composition of claim 34, wherein each gene in the viral vector construct is operably linked to a respective promoter.

40. The pharmaceutical composition of claim 39, wherein the promoter is a constitutive promoter or cardiac specific promoter.

41. The pharmaceutical composition of claim 40, wherein the constitutive promoter is selected from the group consisting of cytomegalovirus (CMV), polyoma, Adenovirus 2, and Simian Virus 40; and the phosphoglycerate kinase (PGK) gene, CAG promoter, and the cardiac promoter is selected from the group consisting of brain natriuretic brain (BNP) promoter, the cardiac tropinin T (cTnT) promoter, and the myosin light chain 2v (MLC-2v) promoter fused to a minimal cytomegalovirus (CMV) enhancer.

42. The pharmaceutical composition of claim 34, wherein each respective gene is operably linked to a polyadenylation signal.

43. The pharmaceutical composition of claim 42, wherein the polyadenylation signal is selected from the group consisting of a bovine growth hormone polyadenylation signal (BGHpA), a SV40 polyadenylation signal or a rabbit beta-globin polyadenylation signal.

44. The pharmaceutical composition of claim 34, wherein the adenoviral vector construct or the adeno-associated viral vector construct is single-stranded or double-stranded.

45. The pharmaceutical composition of claim 34, wherein the adenoviral vector construct comprises an adenoviral vector or adeno-associated viral (AAV) vector that is a self-complementary AAV.

46. The pharmaceutical composition of claim 34, wherein the adenoviral vector construct or the adeno-associated viral vector (AAV) construct has a serotype is selected from the group consisting of AAV1, AAV2, AAV3, AAV4, AAV5, AAV6, AAV7, AAV8, AAV9, AAV10, AAV11, or AAV12 and a hybrid serotype thereof.

47. The pharmaceutical composition of claim 39, wherein the vector is an adeno-associated viral vector construct (AAV), further comprising a first AAV inverted terminal repeat (ITR) located upstream of the promoter and a second AAV ITR located downstream of the polyadenylation signal.

48. The pharmaceutical composition of claim 44, wherein the adeno-associated viral vector construct (AAV) is a virus particle or a helper dependent, “gutless” adenoviral vector.

49. A population of cells selected from the group consisting of stem cells, cardiomyocytes, fibroblasts and skeletal muscle cells engineered to express a hyperpolarization-activated cyclic nucleotide-gated (HCN) channel or a biologically active fragment or variant thereof and a voltage gated sodium SkM1 channel or a biologically active fragment or variant thereof.

50. A method for treating sinoatrial node dysfunction and/or atrioventricular conduction block in a human or animal subject, comprising administering to an area at, near or remote from the site of the sinoatrial node dysfunction and/or atrioventricular conduction block a composition comprising viral vector constructs or a non-viral vector constructs comprising
a gene encoding a biologically active hyperpolarization-activated cyclic nucleotide-gated channel (HCN) selected from the group comprising HCN2, HCN1 channels, HCN4 channels and HCN channels that have activation kinetics similar to HCN2 and cAMP responsiveness similar to HCN2, or a biologically active fragment or variant thereof,
and a gene encoding a biologically active voltage gated sodium channel that has a depolarized inactivation relation similar to SkM1 or a biologically active fragment or variant thereof,
in a therapeutically effect amount and under conditions whereby the vectors transduce a plurality of cells located at or near the area and the encoded HCN and sodium channel genes are translated and expressed thereby treating the sinoatrial node dysfunction and/or atrioventricular conduction block.

51. The method of claim 50, wherein the genes (a) are in different vectors or the same vector; (b) the HCN gene is a member of the group consisting of mouse HCN2 or human HCN2, an isoform selected from the group consisting of mouse HCN1, human HCN1, mouse HCN4 and human HCN4 or biologically active fragments or variants thereof, and HCN2, HCN1 or HCN4 channel genes encoding a protein that is at least 95% identical to human or mouse HCN2, HCN1 and HCN4; and (c) the sodium channel gene is a member of the group consisting of rat SkM1 or human SkM1, a sodium channel gene encoding a neuronal isoform Nav 1.1, 1.2, 1.3, 1.6, 1.7 and 1.9 of the sodium channel gene, and a gene encoding a biologically active Skm1 channel that encodes a protein that is at least 95% identical to human SkM1 or rat SkM1 protein.

52. The method of claim 50, wherein the area of administration is identified by (a) an electrophysiological study performed on the subject, in which multiple left and/or right ventricular sites are stimulated sequentially, and the site that gives the optimal cardiac output is the area at which the composition is administered, or (b) the composition is administered to the subject intramyocardially or by infusion into the coronary vasculature, and the therapeutically effective amount of the composition comprises about 1×10⁹-1×1014 genome copies of the adenoviral vector or the adeno-associated viral vector per kg of body weight of the subject.

53. The method of claim 34, wherein the retroviral vector construct is a member selected from the group consisting of lentiviral LV-CMV-X; LV-EF1α-X; LV-PGK; LV-cTnT-X; LV-cTnI; LV-CAG-X; LV-short troponin I-X; LV-CMV-immediate-early enhancer-ANF-X; LV-CMV-immediate-early enhancer-MLC2v; LV-αMHC-X; LV-MLC2v-X; and the adenovirus vector or the adeno-associated viral vector (AAV) has a serotype is selected from the group consisting of AAV1, AAV2, AAV3, AAV4, AAV5, AAV6, AAV7, AAV8, AAV9, AAV10, AAV11, or AAV12 and a hybrid serotype thereof.

54. The method of claim 34, wherein pacemaker activity of the HCN channel is enhanced by co-expressing its beta subunit, MiRP1 either by including the gene for MiRP1 in the same viral vector construct or by further administering a different viral vector construct comprising a gene encoding a biologically active MiRP1 beta subunit.


SEQUENCE LISTING (28 sequences)

SEQ ID NO: 1 - DNA, 3372 bp, Homo sapiens
  misc_feature (1)..(3372): Human HCN2
cggctccgctccgcactgcccggcgccgcctcgccatggacgcgcgcgggggcggcgggcggcccggggagagcccgggcgcgagccccacgaccgggccgccgccgccgccgcccccgcgcccccccaaacagcagccgccgccgccgccgccgcccgcgccccccccgggccccgggcccgcgcccccccagcacccgccccgggccgaggcgttgcccccggaggcggcggatgagggcggcccgcggggccggctccgcagccgcgacagctcgtgcggccgccccggcaccccgggcgcggcgagcacggccaagggcagcccgaacggcgagtgcgggcgcggcgagccgcagtgcagccccgcggggcccgagggcccggcgcgggggcccaaggtgtcgttctcgtgccgcggggcggcctcggggcccgcgccggggccggggccggcggaggaggcgggcagcgaggaggcgggcccggcgggggagccgcgcggcagccaggccagcttcatgcagcgccagttcggcgcgctcctgcagccgggcgtcaacaagttctcgctgcggatgttcggcagccagaaggccgtggagcgcgagcaggagcgcgtcaagtcggcgggggcctggatcatccacccgtacagcgacttcaggttctactgggacttcaccatgctgctgttcatggtgggaaacctcatcatcatcccagtgggcatcaccttcttcaaggatgagaccactgccccgtggatcgtgttcaacgtggtctcggacaccttcttcctcatggacctggtgttgaacttccgcaccggcattgtgatcgaggacaacacggagatcatcctggaccccgagaagatcaagaagaagtatctgcgcacgtggttcgtggtggacttcgtgtcctccatccccgtggactacatcttccttatcgtggagaagggcattgactccgaggtctacaagacggcacgcgccctgcgcatcgtgcgcttcaccaagatcctcagcctcctgcggctgctgcgcctctcacgcctgatccgctacatccatcagtgggaggagatcttccacatgacctatgacctggccagcgcggtgatgaggatctgcaatctcatcagcatgatgctgctgctctgccactgggacggctgcctgcagttcctggtgcctatgctgcaggacttcccgcgcaactgctgggtgtccatcaatggcatggtgaaccactcgtggagtgaactgtactccttcgcactcttcaaggccatgagccacatgctgtgcatcgggtacggccggcaggcgcccgagagcatgacggacatctggctgaccatgctcagcatgattgtgggtgccacctgctacgccatgttcatcggccacgccactgccctcatccagtcgctggactcctcgcggcgccagtaccaggagaagtacaagcaggtggagcagtacatgtccttccacaagctgccagctgacttccgccagaagatccacgactactatgagcaccgttaccagggcaagatgtttgacgaggacagcatcctgggcgagctcaacgggcccctgcgggaggagatcgtcaacttcaactgccggaagctggtggcctccatgccgctgttcgccaacgccgaccccaacttcgtcacggccatgctgaccaagctcaagttcgaggtcttccagccgggtgactacatcatccgcgaaggcaccatcgggaagaagatgtacttcatccagcacggcgtggtcagcgtgctcactaagggcaacaaggagatgaagctgtccgatggctcctacttcggggagatctgcctgctcacccggggccgccgcacggcgagcgtgcgggctgacacctactgccgcctctattcgctgagcgtggacaacttcaacgaggtgctggaggagtaccccatgatgcggcgcgccttcgagacggtggccatcgaccgcctggaccgcatcggcaagaagaattccatcctcctgcacaaggtgcagcatgacctcaactcgggcgtattcaacaaccaggagaacgccatcatccaggagatcgtcaagtacgaccgcgagatggtgcagcaggccgagctgggtcagcgcgtgggcctcttcccgccgccgccgccgccgccgcaggtcacctcggccatcgccacgctgcagcaggcggcggccatgagcttctgcccgcaggtggcgcggccgctcgtggggccgctggcgctcggctcgccgcgcctcgtgcgccgcccgcccccggggcccgcacctgccgccgcctcacccgggcccccgccccccgccagccccccgggcgcgcccgccagcccccgggcaccgcggacctcgccctacggcggcctgcccgccgccccccttgctgggcccgccctgcccgcgcgccgcctgagccgcgcgtcgcgcccactgtccgcctcgcagccctcgctgcctcacggcgcccccggccccgcggcctccacacgcccggccagcagctccacaccgcgcttggggcccacgcccgctgcccgggccgccgcgcccagcccggaccgcagggactcggcctcacccggcgccgccggcggcctggacccccaggactccgcgcgctcgcgcctctcgtccaacttgtgaccctcgccgaccgccccgcgggcccaggcgggccgggggcggggccgtcatccagaccaaagccatgccattgcgctgccccggccgccagtccgcccagaagccatagacgagacgtaggtagccgtagttggacggacgggcagggccggcggggcagccccctccgcgcccccggccgtcccccctcatcgccccgcgcccacccccatcgcccctgcccccggcggcggcctcgcgtgcgagggggctcccttcacctcggtgcctcagttcccccagctgtaagacagggacggggcggcccagtggctgagaggagccggctgtggagccccgcccgccccccaccctctaggtggcccccgtccgaggaggatcgttttctaagtgcaatacttggcccgccggcttcccgctgcccccatcgcgctcacgcaataaccggcccggcccccgtccgcgcgcgtcccccggtgacctcggggagcagcaccccgcctccctccagcactggcaccgagaggcaggcctggctgcgcagggcgcgggggggaggctggggtcccgccgccgtgatgaatgtactgacgagccgaggcagcagtgcccccaccgtggccccccacgccccattaacccccacacccccattccgcgcaataaa

SEQ ID NO: 2 - DNA, 809 bp, Homo sapiens
  misc_feature (1)..(809): Human KCNE2
gtaaggtgaaggtgcccagcaggctgaggcttgtgtgcaacccagaagagagctcgctaacgccagcaagaaggttcagaacagcctggctttggaaaggaatttcatcctgcccacacactgcatagcaggagggaagcatgtctactttatccaatttcacacagacgctggaagacgtcttccgaaggatttttattacttatatggacaattggcgccagaacacaacagctgagcaagaggccctccaagccaaagttgatgctgagaacttctactatgtcatcctgtacctcatggtgatgattggaatgttctctttcatcatcgtggccatcctggtgagcactgtgaaatccaagagacgggaacactccaatgacccctaccaccagtacattgtagaggactggcaggaaaagtacaagagccaaatcttgaatctagaagaatcgaaggccaccatccatgagaacattggtgcggctgggttcaaaatgtccccctgataagggagaaaggcaccaagctaacatctgacgtccagacatgaagagatgccagtgccacgaggcaaatccaaattgtctttgcttagaagaaagtgagttccttgctctctgttgagaattttcatggagattatgtggttggccaataaagatagatgacatttcaatctcagtgatttatgcttgcttgttggagcaatattttgtgctgaagacctcttttactttccgggcaagtgaatgtcattttaatcaatatcaatgatgaaaataaagccaaatttgaagaaaaaa

SEQ ID NO: 3 - DNA, 6957 bp, Rattus norvegicus
  misc_feature (1)..(6957): Rat SkM1
acggcacccggggctcgtccgccaccgctcttctctgcttctggccaacagctgaagtcacgcagttcctggagtctgaacccaggagacagttcgggacactggggcgttggccccaagctcctcgggctggagaagatctggttcactttctgttgatcccgaaggtggcacaatctattgctgagaagagaacactgcggggcctggagtgggggaatgcaccgtgcccagtgtcctgggcatcagcacgcccaggtcttgcaggcacatctcccagccccaacaagaccaggacacccacctccctgacccaactcctgggcaggcagagagtgaggagtacacagtagaccccgggcattggtgaccacccaaaacccaagggctgcgggctgtagcggctctgttacctaccaccctctggcccctgagcccagaatgcaaggatggccagctcatctctgcccaacctggtccccccgggtccccactgcctgcgccccttcaccccagagtccctggcagccatagagcagcgggcggtggaggaggaggcccggctgcagcggaacaagcagatggagattgaggagcctgagcggaagccacgcagtgacctggaagctggcaagaacctcccactcatctatggggaccccccacccgaagtcattggcatccccctggaggacctggatccttactacagtgacaagaagaccttcattgtgctcaacaaaggaaaggccatcttccgattctctgccacgcctgccctctacctgctgagccccttcagcatcgtcaggagggtggctatcaaggtgctcattcacgcgctgttcagcatgtttatcatgatcaccatcctgaccaactgtgtgttcatgaccatgagcaatccgccttcttggtccaaacacgtggagtacaccttcacggggatctatacctttgagtccctcattaagatgctggcccgaggcttttgcattgatgacttcacattcctccgagacccctggaactggctggacttcagtgtcatcacaatggcgtatgtgacagagtttgtggacttgggcaacatctcagccctgaggaccttccgtgtgctgcgggccctgaagaccatcacggttatcccagggctgaagacaattgtgggagccctgatccagtctgtgaaaaagctgtcggatgtgatgatcctcactgtcttctgcctgagtgtctttgccctggtggggctgcagcttttcatgggaaacctgcgtcagaagtgcgtgcgttggcccccgcccatgaatgacaccaacaccacgtggtatggcaatgacacttggtacagcaatgacacttggtacggcaatgacacttggtacatcaatgacacttggaacagccaggagagctgggccggcaactctacctttgactgggaggcctacatcaatgacgaagggaacttctatttcttggagggctccaatgatgctctgctctgtgggaatagcagtgatgctgggcactgccctgagggctacgaatgcataaaggctgggcggaaccccaactatggctacaccagctatgacaccttcagctgggctttcctggctctcttccggctcatgacgcaggactactgggagaaccttttccagctgaccctacgagctgctggcaagacctacatgatcttcttcgtggtcatcatcttcctgggctccttctacctcatcaatctgatcctggccgtggtggccatggcgtacgctgagcagaatgaggctaccctggccgaagaccaggagaaagaggaggagttccaacagatgcttgagaaatacaaaaaacatcaggaggaactggaaaaggctaaggctgcccaggctctggaaagtggagaggaggcagatggggacccaacccacaacaaagactgcaatgggagcctggatgcatccggggagaaggggcccccaaggccaagctgcagcgcagacagtgccatctcagatgctatggaggagctggaagaggcccatcagaagtgcccaccgtggtggtacaagtgtgcacacaaagtcctcatctggaactgctgtgccccgtgggtgaagttcaaacatataatctacctgatcgtcatggacccctttgtggacctgggtatcaccatctgcattgtgctcaacaccctcttcatggccatggagcactaccccatgaccgagcactttgacaacgtgctctccgtgggcaacttggtcttcacaggcatcttcactgcggagatggtgttgaagctgattgccatggacccctacgagtatttccaacagggctggaacatctttgacagtttcatcgtcaccctcagcctggtggagctgggcctggccaacgtacaggggctgtcagtgctccgttccttccgcctgctgcgtgtcttcaagctggccaagtcatggccaacactcaacatgctcatcaaaatcattggcaactcagtgggcgcgctgggcaacctgaccctggtgctggccatcatcgtcttcatcttcgccgtggtgggcatgcagctgttcggcaagagctataaggagtgtgtgtgcaagatcgcctcagactgcaacctgcctcgctggcacatgaacgacttcttccactccttcctcatcgtcttccgcatcctctgcggggaatggatcgagaccatgtgggactgcatggaggtggccggccaggccatgtgcctcaccgtcttcctcatggtcatggtcattggcaacctggtggtcctgaatctgttcctggctctcctgctgagttccttcagtgctgacagcctggcggcctcggatgaggatggcgagatgaacaacctacagattgccatcgggcgtatcaagtggggcatcggttttgccaaaaccttcctcctggggctgttgcgtggcaagatcctgagccccaaggaaataatactcagcctcggtgagcccgggggtgccggggaaaatgccgaggagagtactcccgaggatgagaagaaggagccgccaccggaagataaggagctgaaagacaatcatatcctgaaccacgtgggcctgaccgatggcccccgctccagcatcgagctggaccaccttaacttcatcaacaacccctacctcaccatccaggtgcccattgcctccgaggagtctgacctggagatgcccacagaggaggagacagacgccttctcggagcctgaggatatcaagaagcccctacagcccctctacgacgggaactcctccgtctgcagcacagctgactacaagccccctgaagaggaccccgaggagcaggctgaggagaaccccgagggggagcagcctgaggaatgcttcacggaagcctgtgtgaagcgctgcccctgcctctatgtggacatctcccagggccgtgggaaaatgtggtggacactgcgcagggcctgtttcaagattgttgagcacaactggtttgagaccttcattgtcttcatgatcttgctcagcagtggagccctggccttcgaggacatctacattgaacagcgacgagtcatccgcaccatcctggaatacgccgacaaggtcttcacctacatcttcatcctggagatgttgctaaagtgggtcgcctacggcttcaaggtgtatttcaccaatgcctggtgctggctcgacttcctcatcgtggacgtctccatcatcagcctggtggcaaactggctgggctactctgagctggggcccatcaaatccctgcgcacactccgggccctgcgacctctgagggcactgtctcgatttgagggtatgagggtggtggtgaacgccctcctgggagctatcccctccatcatgaacgtcctcctcgtctgcctcatcttctggctcatcttcagcatcatgggggtcaacttgtttgctggaaagttctactactgcgtcaacaccaccacctctgagagattcgacatctcggtggtcaacaacaagtccgagtctgagagcctcatgtatacaggccaggtccgctggatgaatgtcaaggtcaactacgacaacgtgggtctgggctatctctccctccttcaggtggccacattcaagggttggatggatatcatgtatgcagctgtggactcccgggagaaagaggagcagccacactatgaggtgaacctctacatgtacctctacttcgtcatcttcatcatcttcggctccttcttcaccctcaacctcttcatcggtgtcatcatcgacaacttcaaccaacagaagaagaagtttggagggaaagacatcttcatgacagaggaacagaagaaatactacaatgcgatgaagaagcttggctccaagaagccccagaagccaattccccggcctcagaacaagatccagggcatggtgtacgacttcgtgacgaagcaggtgtttgacatctccatcatgatcctcatctgcctcaacatggttactatgatggtggagacagatgaccagagccagctcaaggtggacatcctgtacaacatcaacatggtcttcattatcatcttcacaggggagtgtgtgctgaagatgtttgccctacgccattactacttcaccattggctggaacatctttgactttgtggttgtcatcttgtccatcgtgggccttgcgctctctgacttgatacagaaatactttgtgtcacccacgctgttccgtgtgatccgcttggctcggatcgggcgtgtcctgcgtctgatccgcggggccaagggcatccggacgctgctgttcgccctgatgatgtccctgcccgccctcttcaacatcggcctccttctctttctggtcatgttcatctactcgatcttcggcatgtctaacttcgcctacgtcaagaaagagtcaggcatcgacgacatgtttaactttgagacctttggcaacagtatcatctgcctcttcgagatcaccacgtcagccggctgggacgggcttctgaaccccatcctcaacagtgggcccccagactgtgacccgacgttggagaacccgggtaccaatgtcaggggggactgcggcaacccttccatcggcatctgtttcttctgcagctacatcatcatctccttcctcatcgtggtcaacatgtacattgctatcatcctggagaatttcaacgtggccaccgaagagagcagcgagcccctcagtgaggatgacttcgagatgttctatgagacctgggagaagttcgacccggacgccactcagttcatcgactacagccgcctctcggactttgtggacaccctgcaggagccactgaaaatcgccaaacccaataagatcaagcttatcacgttagacctgcccatggtgcccggcgacaagatccactgcttggacatcctctttgccctgaccaaagaggtactgggtgactctggggagatggacgccctcaagcagaccatggaggagaagtttatggcagctaacccttccaaggtctcctatgagcccatcaccaccaccctcaagaggaagcaggaggaggtgtgtgctatcaaaatccagagggcctaccgccgccacctgctgcagcgctccgtgaagcaggcctcttacatgtaccgtcacagccaggacggcaatgacgacggggcccccgagaaggagggattgcttgccaacaccatgaacaagatgtatggccatgagaaagagggtgatggtgtgcagagccagggggaggaagagaaggcctctacagaggatgctggacccaccgtggagcccgaacccaccagcagctcagatactgccctgactccttctcctccaccactacccccctcatcatcaccgccacaagggcagacagttcgcccaggggtcaaagagtctcttgtctagatggcagcgtcaaggctgcacgcagagttggaggggaagggctttgagatgggggctatacaggacttgacacctgggctgaggggtatggaaattgtgctcagggttcaggtgggtcggaagcccctgctgcctgatttcatctttacgatgctctgacattctgttggccaggggaggccaggacatctgaatgctgtttccttcttgggggagagccagcataccttggaggggttggccaccttcctgtcactactttaagggcccaggaccccagctctagccaagacaaagacgaatcttaacccccaaacctacacctgacccccagagcttagctatccctctcgcccccataattctagggtgcagcttccccctgacagctcaggaatgccctgtaaaaggggaaatggttctggtacagaaccaattggccgctggatccggatgtatcaggctggaaacatgacttctgaaacctttcacccagtcagatccaggccggtgggcagctcccctggctggcccctgggggaagaactaaggaaggattgccctgagaaattacctttctctagattgcgtggatgggtcaaacaggctgggtcctgccagctgcattcatgcccttgagcctgagcgcccacatctgcctggggtcaggttggaagctctctatcaagaatcgcccgttcccgcctccctgccttctttctttgaataccagggctcctgggagccccatcaaacacgaaagactgccttcctcctgtctaatctctgccccccccttccctgccccttccctgcttcctcaagtgactctgagaatgctagaactcagcacacgggagatgagttgatactggggcatgcagtgagaaggctcaccaggcttggctttggtcttctcattgcctgaaagcaagttttcccctgagacagggtttcttttctccgtgtagccctggctgcctgg

SEQ ID NO: 4 - DNA, 7823 bp, Homo sapiens
  misc_feature (1)..(7823): Human SkM1
ccagcaccccggggctgcgcactgcagctccccaggccacccaccacccttctggtctctgagcccaggatgcgaggatggccagaccatctctgtgcaccctggctcgtctgggccctgagtgcttgcgccccttcacccgggagtcactggcagccatagaacagcgggcggtggaggaggaggcccggctgcagcggaataagcagatggagattgaggagcccgaacggaagccacgaagtgacttggaggctggcaagaacctacccatgatctacggagaccccccgccggaggtcatcggcatccccctggaggacctggatccctactacagcaataagaagaccttcatcgtactcaacaagggcaaggccatcttccgcttctccgccacacctgctctctacctgctgagccccttcagcgtagtcaggcgcggggccatcaaggtgctcatccatgcgctgttcagcatgttcatcatgatcaccatcttgaccaactgcgtattcatgaccatgagtgacccgcctccctggtccaagaatgtggagtacaccttcacagggatctacacctttgagtccctcatcaagatactggcccgaggcttctgtgtcgacgacttcacattcctccgggacccctggaactggctggacttcagtgtcatcatgatggcgtacctgacagagtttgtggacttgggcaacatctcagccctgaggaccttccgggtgctgcgggccctcaaaaccatcacggtcatcccagggctgaagacgatcgtgggggccctgatccagtcggtgaagaagctgtcggatgtgatgatcctcactgtcttctgcctgagcgtctttgcgctggtaggactgcagctcttcatgggaaacctgaggcagaagtgtgtgcgctggcccccgccgttcaacgacaccaacaccacgtggtacagcaatgacacgtggtacggcaatgacacatggtatggcaatgagatgtggtacggcaatgactcatggtatgccaacgacacgtggaacagccatgcaagctgggccaccaacgatacctttgattgggacgcctacatcagtgatgaagggaacttctacttcctggagggctccaacgatgccctgctctgtgggaacagcagtgatgctgggcactgccctaagggttatgagtgcatcaagaccgggcggaaccccaactatggctacaccagctatgacaccttcagctgggccttcttggctctcttccgcctcatgacacaggactattgggagaacctcttccagctgacccttcgagcagctggcaagacctacatgatcttcttcgtggtcatcatcttcctgggctctttctacctcatcaatctgatcctggccgtggtggccatggcatatgccgagcagaatgaggccaccctggccgaggataaggagaaagaggaggagtttcagcagatgcttgagaagttcaaaaagcaccaggaggagctggagaaggccaaggccgcccaagctctggaaggtggggaggcagatggggacccagcccatggcaaagactgcaatggcagcctggacacatcgcaaggggagaagggagccccgaggcagagcggcagcggagacagcggcatctccgacgccatggaagaactggaagaggcccaccaaaagtgcccaccatggtggtacaagtgcgcccacaaagtgctcatatgggactgctgcgccccgtggctgaagttcaagaacatcatccacctgatcgtcatggacccgttcgtggacctgggcatcaccatctgcatcgtgctcaacaccctcttcatggccatggaacattaccccatgacggagcactttgacaacgtgctcactgtgggcaacctggtcttcacaggcatcttcacagcagagatggttctgaagctgattgccatggacccctacgagtatttccagcagggttggaatatcttcgacagcatcatcgtcaccctcagcctggtagagctaggcctggccaacgtacagggactgtctgtgctacgctccttccgtctgctgcgggtcttcaagctggccaagtcgtggccaacgctgaacatgctcatcaagatcattggcaattcagtgggggcgctgggtaacctgacgctggtgctggctatcatcgtgttcatcttcgccgtggtgggcatgcagctgtttggcaagagctacaaggagtgcgtgtgcaagattgccttggactgcaacctgccgcgctggcacatgcatgatttcttccactccttcctcatcgtcttccgcatcctgtgcggggagtggatcgagaccatgtgggactgcatggaggtggccggccaagccatgtgcctcaccgtcttcctcatggtcatggtcatcggcaatcttgtggtcctgaacctgttcctggctctgctgctgagctccttcagcgccgacagtctggcagcctcggatgaggatggcgagatgaacaacctgcagattgccatcgggcgcatcaagttgggcatcggctttgccaaggccttcctcctggggctgctgcatggcaagatcctgagccccaaggacatcatgctcagcctcggggaggctgacggggccggggaggctggagaggcgggggagactgcccccgaggatgagaagaaggagccgcccgaggaggacctgaagaaggacaatcacatcctgaaccacatgggcctggctgacggccccccatccagcctcgagctggaccaccttaacttcatcaacaacccctacctgaccatacaggtgcccatcgcctccgaggagtccgacctggagatgcccaccgaggaggaaaccgacactttctcagagcctgaggatagcaagaagccgccgcagcctctctatgatgggaactcgtccgtctgcagcacagctgactacaagccccccgaggaggaccctgaggagcaggcagaggagaaccccgagggggagcagcctgaggagtgcttcactgaggcctgcgtgcagcgctggccctgcctctacgtggacatctcccagggccgtgggaagaagtggtggactctgcgcagggcctgcttcaagattgtcgagcacaactggttcgagaccttcattgtcttcatgatcctgctcagcagtggggctctggccttcgaggacatctacattgagcagcggcgagtcattcgcaccatcctagaatatgccgacaaggtcttcacctacatcttcatcatggagatgctgctcaaatgggtggcctacggctttaaggtgtacttcaccaacgcctggtgctggctcgacttcctcatcgtggatgtctccatcatcagcttggtggccaactggctgggctactcggagctgggacccatcaaatccctgcggacactgcgggccctgcgtcccctgagggcactgtcccgattcgagggcatgagggtggtggtgaacgccctcctaggcgccatcccctccatcatgaatgtgctgcttgtctgcctcatcttctggctgatcttcagcatcatgggtgtcaacctgtttgccggcaagttctactactgcatcaacaccaccacctctgagaggttcgacatctccgaggtcaacaacaagtctgagtgcgagagcctcatgcacacaggccaggtccgctggctcaatgtcaaggtcaactacgacaacgtgggtctgggctacctctccctcctgcaggtggccaccttcaagggttggatggacatcatgtatgcagccgtggactcccgggagaaggaggagcagccgcagtacgaggtgaacctctacatgtacctctactttgtcatcttcatcatctttggctccttcttcaccctcaacctcttcattggcgtcatcattgacaacttcaaccagcagaagaagaagttaggggggaaagacatctttatgacggaggaacagaagaaatactataacgccatgaagaagcttggctccaagaagcctcagaagccaattccccggccccagaacaagatccagggcatggtgtatgacctcgtgacgaagcaggccttcgacatcaccatcatgatcctcatctgcctcaacatggtcaccatgatggtggagacagacgaccagagccagctcaaggtggacatcctgtacaacatcaacatgatcttcatcatcatcttcacaggggagtgcgtgctcaagatgctcgccctgcgccagtactacttcaccgttggctggaacatctttgacttcgtggtcgtcatcctgtccattgtgggccttgccctctctgacctgatccagaagtacttcgtgtcacccacgctgttccgtgtgatccgcctggcgcggattgggcgtgtcctgcggctgatccgcggggccaagggcatccggacgctgctgttcgccctcatgatgtcgctgcctgccctcttcaacatcggcctcctcctcttcctggtcatgttcatctactccatcttcggcatgtccaactttgcctacgtcaagaaggagtcgggcatcgatgatatgttcaacttcgagaccttcggcaacagcatcatctgcctgttcgagatcaccacgtcggccggctgggacgggctcctcaaccccatcctcaacagcgggcccccagactgtgaccccaacctggagaacccgggcaccagtgtcaagggtgactgcggcaacccctccatcggcatctgcttcttctgcagctatatcatcatctccttcctcatcgtggtcaacatgtacatcgccatcatcctggagaacttcaatgtggccacagaggagagcagcgagccccttggtgaagatgactttgagatgttctacgagacatgggagaagttcgaccccgacgccacccagttcatcgcctacagccgcctctcagacttcgtggacaccctgcaggaaccgctgaggattgccaagcccaacaagatcaagctcatcacactggacttgcccatggtgccaggggacaagatccactgcctggacatcctctttgccctgaccaaagaggtcctgggtgactctggggaaatggacgccctcaagcagaccatggaggagaagttcatggcagccaacccctccaaggtgtcctacgagcccatcaccaccaccctcaagaggaagcacgaggaggtgtgcgccatcaagatccagagggcctaccgccggcacctgctacagcgctccatgaagcaggcatcctacatgtaccgccacagccacgacggcagcggggatgacgcccctgagaaggaggggctgcttgccaacaccatgagcaagatgtatggccacgagaatgggaacagcagctcgccaagcccggaggagaagggcgaggcaggggacgccggacccactatggggctgatgcccatcagcccctcagacactgcctggcctcccgcccctcccccagggcagactgtgcgcccaggtgtcaaggagtctcttgtctagcaggcagcatcggggtggccccactgagtctcggcatagtccccagagctcccccgtggtgcctgcacacagagtgagggaggagggctttgaatctgggactgtgcctggctccctgatgggggacaggatttggccacactggggctgacacccaggcccgagcgcctgcgttcccagaccatgggaaatgggaattgcgctcaggggctccatgctgggtctgaggcccctgcctccaagatttaacctgcaagttgctctgacctcctctgggccctgtcgcccctccttttggcctgggggaggtcagaacattcgaatctctgcccctcacttgaggaggagctggcctgcggtggagggatcagttgccccccatcaccagagtcttaagggtcactggcctctccccaggaagtgggctcagacccccctcagccccagcccagacaaagatgtcttaacctcagggagtgcagacacctaaccccagggcactgccagcccaccccctttgactctggggtgcagcttcacccaccaggccagctcaggaattccctggaaaagggaaatgtgactggttcagaaatagctcctcaaagcctcaaaacctgattggccactggatcctgctgctttgggctgggatggtgactcctgaaacctcttcctaggccacgtccaggtccgtagctcccctggctggctcctaggggaagagcagaaggaaggatgccacttgggaatgaattgtccttttctaggaagcacgggggagtgagacaggctgggtcctgccagctggatcgctgcacatggcctgagcatccagacctgagcgggagtcagggacctgctgctcagtaagaagattctcgccccttccctctctccctgcctcactcctccgtgagcaccaccagggctccaggagcctcatccagcctcagagatctcccttctcatctccccacgccccgtctctttctcacctttcccacctctctccccaaagtgatcctaagaatgtacagttgagctcaggttagatatttcgaccctggggcgtgcagcagggaaggcccaactggttcaggctcaaccttccaacttcctgtggcctgaagaagcacttctgctgcatcgctgttctgggcatggcagggccaggcctctgctggctcaggaggaggggtgagagacctgctcaggcgtcgctggatttattcacttgtgtgtgtacctgtggctgtgtgtctgcttgtatgcttttataggcctgtgtgtatagctgtgtgtgtgttcaagtgcgtgactgtatgtgtgtgtgtgaaccactgtgtgaaccactgtgtactggagcctgcattatgcacgtgtctgggtatctttgtatatatgtgtatatatgtgtgccctggactgtttcaaggtccatggagtacggctggtgtgtcatactgtgcaggcctgtccctgggagtgttcccgtgcctgggagagtggacctgtgctgtgagtgtgtggatgcgtgtgaacgcatgtggtaaggtgtgtactcagggcattctgttggcctaagtgcctcttctttttcttcttgtttctcatgaaaagtttgattaaaattcaggaagcagcaaaaccttcaaaacaagacatgtatgtgtgcttgagtgtgtgaacacgtgtgtgtgtgtgcacatctacatgccatgcctatgggccagagttgtctttattgtccaccatgctctctcacctgcttcccagtcctgcctgaacagccctctctctcactcccctctcctccccttcctgtttctcgttgtcacacccatggcctcagccctgctccctgcctcctgcctatgtctcctctatggaaggaggcctccactccttccatctcttccttcagaagtttcgtctaatgggggcagtctccccttcctggcacattgcccctctgccttgccctcctgggccctgggctggcacagcccctggagcctcagaaatctgtttgattggatattctcctcggactgtgtgcaggttgcagaggaagagtagatgagccgggtccggcctctccctgcctgtggcccctcccctgcagacggatgcccattcctgcctggtccagtggggaacaggtcccacgccaggccagcaggcgggctcctttgtacagttcttacaataaaccctccttggtgcctctgg

SEQ ID NO: 5 - protein, 863 aa, Mus musculus
  misc_feature (1)..(863): Mus musculus amino acid sequence for hyperpolarization-activated cation channel HAC1
MDARGGGGRPGDSPGTTPAPGPPPPPPPPAPPQPQPPPAPPPNPTTPSHPESADEPGPRARLCSRDSACTPGAAKGGANGECGRGEPQCSPEGPARGPKVSFSCRGAASGPSAAEEAGSEEAGPAGEPRGSQASFLQRQFGALLQPGVNKFSLRMFGSQKAVEREQERVKSAGAWIIHPYSDFRFYWDFTMLLFMVGNLIIIPVGITFFKDETTAPWIVFNVVSDTFFLMDLVLNFRTGIVIEDNTEIILDPEKIKKKYLRTWFVVDFVSSIPVDYIFLIVEKGIDSEVYKTARALRIVRFTKILSLLRLLRLSRLIRYIHQWEEIFHMTYDLASAVMRICNLISMMLLLCHWDGCLQFLVPMLQDFPSDCWVSINNMVNHSWSELYSFALFKAMSHMLCIGYGRQAPESMTDIWLTMLSMIVGATCYAMFIGHATALIQSLDSSRRQYQEKYKQVEQYMSFHKLPADFRQKIHDYYEHRYQGKMFDEDSILGELNGPLREEIVNFNCRKLVASMPLFANADPNFVTAMLTKLKFEVFQPGDYIIREGTIGKKMYFIQHGVVSVLTKGNKEMKLSDGSYFGEICLLTRGRRTASVRADTYCRLYSLSVDNFNEVLEEYPMMRRAFETVAIDRLDRIGKKNSILLHKVQHDLSSGVFNNQENAIIQEIVKYDREMVQQAELGQRVGLFPPPPPPQVTSAIATLQQAVAMSFCPQVARPLVGPLALGSPRLVRRAPPGPLPPAASPGPPAASPPAAPSSPRAPRTSPYGVPGSPATRVGPALPARRLSRASRPLSASQPSLPHGVPAPSPAASARPASSSTPRLGPAPTARTAAPSPDRRDSASPGAASGLDPLDSARSRLSSNL

SEQ ID NO: 6 - DNA, 3102 bp, Mus musculus
  misc_feature (1)..(3102): Mus musculus nucleic acid sequence encoding mRNA sequence for hyperpolarization-activated cation channel HAC1
ccgctccgctccgcactgcccggcgccgcctcgccatggatgcgcgcgggggcggcgggcggccgggcgatagtccgggcacgacccctgcgccggggccgccgccaccgccgccgccgcccgcgccccctcagcctcagccaccacccgcgccacccccgaaccccacgaccccctcgcacccggagtcggcggacgagcccggcccgcgcgcccggctctgcagccgcgacagcgcctgcacccctggcgcggccaagggcggcgcgaatggcgagtgcgggcgcggggagccgcagtgcagccccgagggccccgcgcgcggccccaaggtttcgttctcatgccgcggggcggcctccgggccctcggcggccgaggaggcgggcagcgaggaggcgggcccggcgggtgagccgcgcggcagccaggctagcttcctgcagcgccaattcggggcgcttctgcagcccggcgtcaacaagttctccctgcggatgttcggcagccagaaggccgtggagcgcgagcaggaacgcgtgaagtcggcgggggcctggatcatccacccctacagcgacttcaggttctactgggacttcaccatgctgttgttcatggtgggaaatctcattatcattcccgtgggcatcactttcttcaaggacgagaccaccgcgccctggatcgtcttcaacgtggtctcggacactttcttcctcatggacttggtgttgaacttccgcaccggcattgttattgaggacaacacggagatcatcctggaccccgagaagataaagaagaagtacttgcgtacgtggttcgtggtggacttcgtgtcatccatcccggtggactacatcttcctcatagtggagaagggaatcgactccgaggtctacaagacagcgcgtgctctgcgcatcgtgcgcttcaccaagatcctcagtctgctgcggctgctgcggctatcacggctcatccgatatatccaccagtgggaagagattttccacatgacctacgacctggcaagtgcagtgatgcgcatctgtaacctgatcagcatgatgctactgctctgccactgggacggttgcctgcagttcctggtgcccatgctgcaagacttccccagcgactgctgggtgtccatcaacaacatggtgaaccactcgtggagcgagctctactcgttcgcgctcttcaaggccatgagccacatgctgtgcatcggctacgggcggcaggcgcccgagagcatgacagacatctggctgaccatgctcagcatgatcgtaggcgccacctgctatgccatgttcattgggcacgccactgcgctcatccagtccctggattcgtcacggcgccaataccaggagaagtacaagcaagtagagcaatacatgtccttccacaaactgcccgctgacttccgccagaagatccacgattactatgaacaccggtaccaagggaagatgtttgatgaggacagcatccttggggaactcaacgggccactgcgtgaggagattgtgaacttcaactgccggaagctggtggcttccatgccgctgtttgccaatgcagaccccaacttcgtcacagccatgctgacaaagctcaaatttgaggtcttccagcctggagattacatcatccgagaggggaccatcgggaagaagatgtacttcatccagcatggggtggtgagcgtgctcaccaagggcaacaaggagatgaagctgtcggatggctcctatttcggggagatctgcttgctcacgaggggccggcgtacggccagcgtgcgagctgacacctactgtcgcctctactcactgagtgtggacaatttcaacgaggtgctggaggaataccccatgatgcggcgtgcctttgagactgtggctattgaccggctagatcgcataggcaagaagaactccatcttgctgcacaaggttcagcatgatctcagctcaggtgtgttcaacaaccaggagaatgccatcatccaggagattgtcaaatatgaccgtgagatggtgcagcaggcagagcttggccagcgtgtggggctcttcccaccaccgccaccaccgcaggtcacatcggccattgccaccctacagcaggctgtggccatgagcttctgcccgcaggtggcccgcccgctcgtggggcccctggcgctaggctccccacgcctagtgcgccgcgcgcccccagggcctctgcctcctgcagcctcgccagggccacccgcagcaagccccccggctgcaccctcgagccctcgggcaccgcggacctcaccctacggtgtgcctggctctccggcaacgcgcgtggggcccgcattgcccgcacgtcgcctgagccgcgcctcgcgcccactgtccgcctcgcagccctcgctgccccatggcgtgcccgcgcccagccccgcggcctctgcgcgcccggccagcagctccacgccgcgcctgggacccgcacccaccgcccggaccgccgcgcccagtccggaccgcagggactcagcctcgccgggcgctgccagtggcctcgacccactggactctgcgcgctcgcgcctctcttccaacttgtgacccttgagcgccgccccgcgggccgggcggggccgtcatccacaccaaagccatgcctcgcgccgcccgcccgtgcccgtgcagaagccatagagggacgtaggtagcttaggaggcgggcggccctgcgcccggctgtccccccatcgccctgcgcccacccccatcgcccctgccccagcggcggccgcacgggagagggaggggtgcgatcacctcggtgcctcagccccaacctgggacagggacagggcggccctggccgaggacctggctgtgccccgcatgtgcggtggcctccgaggaagaatatggatcaagtgcaatacacggccaagccggcgtgggggtgaggctgggtccccggccgtcgccatgaatgtactgacgagccgaggcagcagtggcccccacgccccattaacccacaaccccattccgcgcaataaacgacagcattggcaaaaaaaaaaaa

SEQ ID NO: 7 - protein, 889 aa, Homo sapiens
  misc_feature (1)..(889): amino acid sequence for  the protein Homo sapiens hyperpolarization-activated, cyclic nucleotide-gated channel 2 (HCN2)
MDARGGGGRPGESPGASPTTGPPPPPPPRPPKQQPPPPPPPAPPPGPGPAPPQHPPRAEALPPEAADEGGPRGRLRSRDSSCGRPGTPGAASTAKGSPNGECGRGEPQCSPAGPEGPARGPKVSFSCRGAASGPAPGPGPAEEAGSEEAGPAGEPRGSQASFMQRQFGALLQPGVNKFSLRMFGSQKAVEREQERVKSAGAWIIHPYSDFRFYWDFTMLLFMVGNLIIIPVGITFFKDETTAPWIVFNVVSDTFFLMDLVLNFRTGIVIEDNTEIILDPEKIKKKYLRTWFVVDFVSSIPVDYIFLIVEKGIDSEVYKTARALRIVRFTKILSLLRLLRLSRLIRYIHQWEEIFHMTYDLASAVMRICNLISMMLLLCHWDGCLQFLVPMLQDFPRNCWVSINGMVNHSWSELYSFALFKAMSHMLCIGYGRQAPESMTDIWLTMLSMIVGATCYAMFIGHATALIQSLDSSRRQYQEKYKQVEQYMSFHKLPADFRQKIHDYYEHRYQGKMFDEDSILGELNGPLREEIVNFNCRKLVASMPLFANADPNFVTAMLTKLKFEVFQPGDYIIREGTIGKKMYFIQHGVVSVLTKGNKEMKLSDGSYFGEICLLTRGRRTASVRADTYCRLYSLSVDNFNEVLEEYPMMRRAFETVAIDRLDRIGKKNSILLHKVQHDLNSGVFNNQENAIIQEIVKYDREMVQQAELGQRVGLFPPPPPPPQVTSAIATLQQAAAMSFCPQVARPLVGPLALGSPRLVRRPPPGPAPAAASPGPPPPASPPGAPASPRAPRTSPYGGLPAAPLAGPALPARRLSRASRPLSASQPSLPHGAPGPAASTRPASSSTPRLGPTPAARAAAPSPDRRDSASPGAAGGLDPQDSARSRLSSNL

SEQ ID NO: 8 - DNA, 3372 bp, Homo sapiens; identical to SEQ ID NO: 1
  misc_feature (1)..(3372): nucleic acid sequence encoding GenBank AF065164.2

SEQ ID NO: 9 - protein, 123 aa, Rattus norvegicus
  misc_feature (1)..(123): amino acid sequence for Rattus norvegicus minK-related peptide 1 mRNA, complete cds
MTTLANLTQTLEDAFKKVFITYMDSWRRNTTAEQQALQARVDAENFYYVILYLMVMIGMFAFIVVAILVSTVKSKRREHSQDPYHQYIVEDWQQKYRSQILHLEDSKATIHENLGATGFTVSP

SEQ ID NO: 10 - DNA, 468 bp, Rattus norvegicus
  misc_feature (1)..(468): nucleic acid sequence encoding the mRNA GenBank AF071003.1
cctgtgaggaatctctcatcctcaagggggaaacatgaccactttagccaacttgacgcagaccctggaggatgccttcaaaaaggttttcattacttatatggacagctggaggaggaacacaacagccgaacaacaggcgctccaggccagagtggatgccgagaacttctactacgtcatcctgtacctcatggtgatgatcggcatgttcgccttcatcgtggtggccatcctggtgagcacggtgaagtcgaagcggcgggagcactcccaggacccgtaccaccagtacatcgtggaggattggcagcagaagtataggagtcagatcttgcatctggaagactccaaggccaccatccatgagaacctgggggcgacggggttcacagtgtcaccctgataaagaacgagagtccatctgcccaggaaggggtgcttctgccgccttgaagccccacttgc

SEQ ID NO: 11 - protein, 123 aa, Homo sapiens
  misc_feature (1)..(123): amino acid sequence for Homo sapiens potassium voltage-gated channel, Isk-related family, member 2 (KCNE2)
MSTLSNFTQTLEDVFRRIFITYMDNWRQNTTAEQEALQAKVDAENFYYVILYLMVMIGMFSFIIVAILVSTVKSKRREHSNDPYHQYIVEDWQEKYKSQILNLEESKATIHENIGAAGFKMSP

SEQ ID NO: 12 - DNA, 809 bp, Homo sapiens; identical to SEQ ID NO: 2
  misc_feature (1)..(809): nucleic acid sequence encoding NCBI Reference Sequence NM_172201.1

SEQ ID NO: 13 - protein, 1840 aa, Rattus norvegicus
  misc_feature (1)..(1840): amino acid sequence for Rattus norvegicus sodium channel, voltage-gated, type IV, alpha subunit (Scn4a)
MASSSLPNLVPPGPHCLRPFTPESLAAIEQRAVEEEARLQRNKQMEIEEPERKPRSDLEAGKNLPLIYGDPPPEVIGIPLEDLDPYYSDKKTFIVLNKGKAIFRFSATPALYLLSPFSIVRRVAIKVLIHALFSMFIMITILTNCVFMTMSNPPSWSKHVEYTFTGIYTFESLIKMLARGFCIDDFTFLRDPWNWLDFSVITMAYVTEFVDLGNISALRTFRVLRALKTITVIPGLKTIVGALIQSVKKLSDVMILTVFCLSVFALVGLQLFMGNLRQKCVRWPPPMNDTNTTWYGNDTWYSNDTWYGNDTWYINDTWNSQESWAGNSTFDWEAYINDEGNFYFLEGSNDALLCGNSSDAGHCPEGYECIKAGRNPNYGYTSYDTFSWAFLALFRLMTQDYWENLFQLTLRAAGKTYMIFFVVIIFLGSFYLINLILAVVAMAYAEQNEATLAEDQEKEEEFQQMLEKYKKHQEELEKAKAAQALESGEEADGDPTHNKDCNGSLDASGEKGPPRPSCSADSAISDAMEELEEAHQKCPPWWYKCAHKVLIWNCCAPWVKFKHIIYLIVMDPFVDLGITICIVLNTLFMAMEHYPMTEHFDNVLSVGNLVFTGIFTAEMVLKLIAMDPYEYFQQGWNIFDSFIVTLSLVELGLANVQGLSVLRSFRLLRVFKLAKSWPTLNMLIKIIGNSVGALGNLTLVLAIIVFIFAVVGMQLFGKSYKECVCKIASDCNLPRWHMNDFFHSFLIVFRILCGEWIETMWDCMEVAGQAMCLTVFLMVMVIGNLVVLNLFLALLLSSFSADSLAASDEDGEMNNLQIAIGRIKWGIGFAKTFLLGLLRGKILSPKEIILSLGEPGGAGENAEESTPEDEKKEPPPEDKELKDNHILNHVGLTDGPRSSIELDHLNFINNPYLTIQVPIASEESDLEMPTEEETDAFSEPEDIKKPLQPLYDGNSSVCSTADYKPPEEDPEEQAEENPEGEQPEECFTEACVKRCPCLYVDISQGRGKMWWTLRRACFKIVEHNWFETFIVFMILLSSGALAFEDIYIEQRRVIRTILEYADKVFTYIFILEMLLKWVAYGFKVYFTNAWCWLDFLIVDVSIISLVANWLGYSELGPIKSLRTLRALRPLRALSRFEGMRVVVNALLGAIPSIMNVLLVCLIFWLIFSIMGVNLFAGKFYYCVNTTTSERFDISVVNNKSESESLMYTGQVRWMNVKVNYDNVGLGYLSLLQVATFKGWMDIMYAAVDSREKEEQPHYEVNLYMYLYFVIFIIFGSFFTLNLFIGVIIDNFNQQKKKFGGKDIFMTEEQKKYYNAMKKLGSKKPQKPIPRPQNKIQGMVYDFVTKQVFDISIMILICLNMVTMMVETDDQSQLKVDILYNINMVFIIIFTGECVLKMFALRHYYFTIGWNIFDFVVVILSIVGLALSDLIQKYFVSPTLFRVIRLARIGRVLRLIRGAKGIRTLLFALMMSLPALFNIGLLLFLVMFIYSIFGMSNFAYVKKESGIDDMFNFETFGNSIICLFEITTSAGWDGLLNPILNSGPPDCDPTLENPGTNVRGDCGNPSIGICFFCSYIIISFLIVVNMYIAIILENFNVATEESSEPLSEDDFEMFYETWEKFDPDATQFIDYSRLSDFVDTLQEPLKIAKPNKIKLITLDLPMVPGDKIHCLDILFALTKEVLGDSGEMDALKQTMEEKFMAANPSKVSYEPITTTLKRKQEEVCAIKIQRAYRRHLLQRSVKQASYMYRHSQDGNDDGAPEKEGLLANTMNKMYGHEKEGDGVQSQGEEEKASTEDAGPTVEPEPTSSSDTALTPSPPPLPPSSSPPQGQTVRPGVKESLV

SEQ ID NO: 14 - DNA, 6957 bp, Rattus norvegicus; identical to SEQ ID NO: 3
  misc_feature (1)..(6957): nucleic acid sequence encoding  NCBI Reference Sequence NM_013178.1

SEQ ID NO: 15 - protein, 1836 aa, Homo sapiens
  misc_feature (1)..(1836): amino acid sequence for Homo sapiens skeletal muscle voltage-dependent sodium channel alpha subunit (SkM1)
MARPSLCTLARLGPECLRPFTRESLAAIEQRAVEEEARLQRNKQMEIEEPERKPRSDLEAGKNLPMIYGDPPPEVIGIPLEDLDPYYSNKKTFIVLNKGKAIFRFSATPALYLLSPFSVVRRGAIKVLIHALFSMFIMITILTNCVFMTMSDPPPWSKNVEYTFTGIYTFESLIKILARGFCVDDFTFLRDPWNWLDFSVIMMAYLTEFVDLGNISALRTFRVLRALKTITVIPGLKTIVGALIQSVKKLSDVMILTVFCLSVFALVGLQLFMGNLRQKCVRWPPPFNDTNTTWYSNDTWYGNDTWYGNEMWYGNDSWYANDTWNSHASWATNDTFDWDAYISDEGNFYFLEGSNDALLCGNSSDAGHCPKGYECIKTGRNPNYGYTSYDTFSWAFLALFRLMTQDYWENLFQLTLRAAGKTYMIFFVVIIFLGSFYLINLILAVVAMAYAEQNEATLAEDKEKEEEFQQMLEKFKKHQEELEKAKAAQALEGGEADGDPAHGKDCNGSLDTSQGEKGAPRQSGSGDSGISDAMEELEEAHQKCPPWWYKCAHKVLIWDCCAPWLKFKNIIHLIVMDPFVDLGITICIVLNTLFMAMEHYPMTEHFDNVLTVGNLVFTGIFTAEMVLKLIAMDPYEYFQQGWNIFDSIIVTLSLVELGLANVQGLSVLRSFRLLRVFKLAKSWPTLNMLIKIIGNSVGALGNLTLVLAIIVFIFAVVGMQLFGKSYKECVCKIALDCNLPRWHMHDFFHSFLIVFRILCGEWIETMWDCMEVAGQAMCLTVFLMVMVIGNLVVLNLFLALLLSSFSADSLAASDEDGEMNNLQIAIGRIKLGIGFAKAFLLGLLHGKILSPKDIMLSLGEADGAGEAGEAGETAPEDEKKEPPEEDLKKDNHILNHMGLADGPPSSLELDHLNFINNPYLTIQVPIASEESDLEMPTEEETDTFSEPEDSKKPPQPLYDGNSSVCSTADYKPPEEDPEEQAEENPEGEQPEECFTEACVQRWPCLYVDISQGRGKKWWTLRRACFKIVEHNWFETFIVFMILLSSGALAFEDIYIEQRRVIRTILEYADKVFTYIFIMEMLLKWVAYGFKVYFTNAWCWLDFLIVDVSIISLVANWLGYSELGPIKSLRTLRALRPLRALSRFEGMRVVVNALLGAIPSIMNVLLVCLIFWLIFSIMGVNLFAGKFYYCINTTTSERFDISEVNNKSECESLMHTGQVRWLNVKVNYDNVGLGYLSLLQVATFKGWMDIMYAAVDSREKEEQPQYEVNLYMYLYFVIFIIFGSFFTLNLFIGVIIDNFNQQKKKLGGKDIFMTEEQKKYYNAMKKLGSKKPQKPIPRPQNKIQGMVYDLVTKQAFDITIMILICLNMVTMMVETDDQSQLKVDILYNINMIFIIIFTGECVLKMLALRQYYFTVGWNIFDFVVVILSIVGLALSDLIQKYFVSPTLFRVIRLARIGRVLRLIRGAKGIRTLLFALMMSLPALFNIGLLLFLVMFIYSIFGMSNFAYVKKESGIDDMFNFETFGNSIICLFEITTSAGWDGLLNPILNSGPPDCDPNLENPGTSVKGDCGNPSIGICFFCSYIIISFLIVVNMYIAIILENFNVATEESSEPLGEDDFEMFYETWEKFDPDATQFIAYSRLSDFVDTLQEPLRIAKPNKIKLITLDLPMVPGDKIHCLDILFALTKEVLGDSGEMDALKQTMEEKFMAANPSKVSYEPITTTLKRKHEEVCAIKIQRAYRRHLLQRSMKQASYMYRHSHDGSGDDAPEKEGLLANTMSKMYGHENGNSSSPSPEEKGEAGDAGPTMGLMPISPSDTAWPPAPPPGQTVRPGVKESLV

SEQ ID NO: 16 - DNA, 7823 bp, Homo sapiens; identical to SEQ ID NO: 4
  misc_feature (1)..(7823): nucleic acid sequence for the corresponding GenBank M81758.1

SEQ ID NO: 17 - DNA, 8133 bp, Homo sapiens
  misc_feature (1)..(8133): Homo sapienssodium channel, voltage-gated, type I, alpha subunit (SCN1A), transcript variant 1
aatgtgcaggatgacaagatggagcaaacagtgcttgtaccaccaggacctgacagcttcaacttcttcaccagagaatctcttgcggctattgaaagacgcattgcagaagaaaaggcaaagaatcccaaaccagacaaaaaagatgacgacgaaaatggcccaaagccaaatagtgacttggaagctggaaagaaccttccatttatttatggagacattcctccagagatggtgtcagagcccctggaggacctggacccctactatatcaataagaaaacttttatagtattgaataaagggaaggccatcttccggttcagtgccacctctgccctgtacattttaactcccttcaatcctcttaggaaaatagctattaagattttggtacattcattattcagcatgctaattatgtgcactattttgacaaactgtgtgtttatgacaatgagtaaccctcctgattggacaaagaatgtagaatacaccttcacaggaatatatacttttgaatcacttataaaaattattgcaaggggattctgtttagaagattttactttccttcgggatccatggaactggctcgatttcactgtcattacatttgcgtacgtcacagagtttgtggacctgggcaatgtctcggcattgagaacattcagagttctccgagcattgaagacgatttcagtcattccaggcctgaaaaccattgtgggagccctgatccagtctgtgaagaagctctcagatgtaatgatcctgactgtgttctgtctgagcgtatttgctctaattgggctgcagctgttcatgggcaacctgaggaataaatgtatacaatggcctcccaccaatgcttccttggaggaacatagtatagaaaagaatataactgtgaattataatggtacacttataaatgaaactgtctttgagtttgactggaagtcatatattcaagattcaagatatcattatttcctggagggttttttagatgcactactatgtggaaatagctctgatgcaggccaatgtccagagggatatatgtgtgtgaaagctggtagaaatcccaattatggctacacaagctttgataccttcagttgggcttttttgtccttgtttcgactaatgactcaggacttctgggaaaatctttatcaactgacattacgtgctgctgggaaaacgtacatgatattttttgtattggtcattttcttgggctcattctacctaataaatttgatcctggctgtggtggccatggcctacgaggaacagaatcaggccaccttggaagaagcagaacagaaagaggccgaatttcagcagatgattgaacagcttaaaaagcaacaggaggcagctcagcaggcagcaacggcaactgcctcagaacattccagagagcccagtgcagcaggcaggctctcagacagctcatctgaagcctctaagttgagttccaagagtgctaaggaaagaagaaatcggaggaagaaaagaaaacagaaagagcagtctggtggggaagagaaagatgaggatgaattccaaaaatctgaatctgaggacagcatcaggaggaaaggttttcgcttctccattgaagggaaccgattgacatatgaaaagaggtactcctccccacaccagtctttgttgagcatccgtggctccctattttcaccaaggcgaaatagcagaacaagccttttcagctttagagggcgagcaaaggatgtgggatctgagaacgacttcgcagatgatgagcacagcacctttgaggataacgagagccgtagagattccttgtttgtgccccgacgacacggagagagacgcaacagcaacctgagtcagaccagtaggtcatcccggatgctggcagtgtttccagcgaatgggaagatgcacagcactgtggattgcaatggtgtggtttccttggttggtggaccttcagttcctacatcgcctgttggacagcttctgccagaggtgataatagataagccagctactgatgacaatggaacaaccactgaaactgaaatgagaaagagaaggtcaagttctttccacgtttccatggactttctagaagatccttcccaaaggcaacgagcaatgagtatagccagcattctaacaaatacagtagaagaacttgaagaatccaggcagaaatgcccaccctgttggtataaattttccaacatattcttaatctgggactgttctccatattggttaaaagtgaaacatgttgtcaacctggttgtgatggacccatttgttgacctggccatcaccatctgtattgtcttaaatactcttttcatggccatggagcactatccaatgacggaccatttcaataatgtgcttacagtaggaaacttggttttcactgggatctttacagcagaaatgtttctgaaaattattgccatggatccttactattatttccaagaaggctggaatatctttgacggttttattgtgacgcttagcctggtagaacttggactcgccaatgtggaaggattatctgttctccgttcatttcgattgctgcgagttttcaagttggcaaaatcttggccaacgttaaatatgctaataaagatcatcggcaattccgtgggggctctgggaaatttaaccctcgtcttggccatcatcgtcttcatttttgccgtggtcggcatgcagctctttggtaaaagctacaaagattgtgtctgcaagatcgccagtgattgtcaactcccacgctggcacatgaatgacttcttccactccttcctgattgtgttccgcgtgctgtgtggggagtggatagagaccatgtgggactgtatggaggttgctggtcaagccatgtgccttactgtcttcatgatggtcatggtgattggaaacctagtggtcctgaatctctttctggccttgcttctgagctcatttagtgcagacaaccttgcagccactgatgatgataatgaaatgaataatctccaaattgctgtggataggatgcacaaaggagtagcttatgtgaaaagaaaaatatatgaatttattcaacagtccttcattaggaaacaaaagattttagatgaaattaaaccacttgatgatctaaacaacaagaaagacagttgtatgtccaatcatacagcagaaattgggaaagatcttgactatcttaaagatgtaaatggaactacaagtggtataggaactggcagcagtgttgaaaaatacattattgatgaaagtgattacatgtcattcataaacaaccccagtcttactgtgactgtaccaattgctgtaggagaatctgactttgaaaatttaaacacggaagactttagtagtgaatcggatctggaagaaagcaaagagaaactgaatgaaagcagtagctcatcagaaggtagcactgtggacatcggcgcacctgtagaagaacagcccgtagtggaacctgaagaaactcttgaaccagaagcttgtttcactgaaggctgtgtacaaagattcaagtgttgtcaaatcaatgtggaagaaggcagaggaaaacaatggtggaacctgagaaggacgtgtttccgaatagttgaacataactggtttgagaccttcattgttttcatgattctccttagtagtggtgctctggcatttgaagatatatatattgatcagcgaaagacgattaagacgatgttggaatatgctgacaaggttttcacttacattttcattctggaaatgcttctaaaatgggtggcatatggctatcaaacatatttcaccaatgcctggtgttggctggacttcttaattgttgatgtttcattggtcagtttaacagcaaatgccttgggttactcagaacttggagccatcaaatctctcaggacactaagagctctgagacctctaagagccttatctcgatttgaagggatgagggtggttgtgaatgcccttttaggagcaattccatccatcatgaatgtgcttctggtttgtcttatattctggctaattttcagcatcatgggcgtaaatttgtttgctggcaaattctaccactgtattaacaccacaactggtgacaggtttgacatcgaagacgtgaataatcatactgattgcctaaaactaatagaaagaaatgagactgctcgatggaaaaatgtgaaagtaaactttgataatgtaggatttgggtatctctctttgcttcaagttgccacattcaaaggatggatggatataatgtatgcagcagttgattccagaaatgtggaactccagcctaagtatgaagaaagtctgtacatgtatctttactttgttattttcatcatctttgggtccttcttcaccttgaacctgtttattggtgtcatcatagataatttcaaccagcagaaaaagaagtttggaggtcaagacatctttatgacagaagaacagaagaaatactataatgcaatgaaaaaattaggatcgaaaaaaccgcaaaagcctatacctcgaccaggaaacaaatttcaaggaatggtctttgacttcgtaaccagacaagtttttgacataagcatcatgattctcatctgtcttaacatggtcacaatgatggtggaaacagatgaccagagtgaatatgtgactaccattttgtcacgcatcaatctggtgttcattgtgctatttactggagagtgtgtactgaaactcatctctctacgccattattattttaccattggatggaatatttttgattttgtggttgtcattctctccattgtaggtatgtttcttgccgagctgatagaaaagtatttcgtgtcccctaccctgttccgagtgatccgtcttgctaggattggccgaatcctacgtctgatcaaaggagcaaaggggatccgcacgctgctctttgctttgatgatgtcccttcctgcgttgtttaacatcggcctcctactcttcctagtcatgttcatctacgccatctttgggatgtccaactttgcctatgttaagagggaagttgggatcgatgacatgttcaactttgagacctttggcaacagcatgatctgcctattccaaattacaacctctgctggctgggatggattgctagcacccattctcaacagtaagccacccgactgtgaccctaataaagttaaccctggaagctcagttaagggagactgtgggaacccatctgttggaattttcttttttgtcagttacatcatcatatccttcctggttgtggtgaacatgtacatcgcggtcatcctggagaacttcagtgttgctactgaagaaagtgcagagcctctgagtgaggatgactttgagatgttctatgaggtttgggagaagtttgatcccgatgcaactcagttcatggaatttgaaaaattatctcagtttgcagctgcgcttgaaccgcctctcaatctgccacaaccaaacaaactccagctcattgccatggatttgcccatggtgagtggtgaccggatccactgtcttgatatcttatttgcttttacaaagcgggttctaggagagagtggagagatggatgctctacgaatacagatggaagagcgattcatggcttccaatccttccaaggtctcctatcagccaatcactactactttaaaacgaaaacaagaggaagtatctgctgtcattattcagcgtgcttacagacgccaccttttaaagcgaactgtaaaacaagcttcctttacgtacaataaaaacaaaatcaaaggtggggctaatcttcttataaaagaagacatgataattgacagaataaatgaaaactctattacagaaaaaactgatctgaccatgtccactgcagcttgtccaccttcctatgaccgggtgacaaagccaattgtggaaaaacatgagcaagaaggcaaagatgaaaaagccaaagggaaataaatgaaaataaataaaaataattgggtgacaaattgtttacagcctgtgaaggtgatgtatttttatcaacaggactcctttaggaggtcaatgccaaactgactgtttttacacaaatctccttaaggtcagtgcctacaataagacagtgaccccttgtcagcaaactgtgactctgtgtaaaggggagatgaccttgacaggaggttactgttctcactaccagctgacactgctgaagataagatgcacaatggctagtcagactgtagggaccagtttcaaggggtgcaaacctgtgattttggggttgtttaacatgaaacactttagtgtagtaattgtatccactgtttgcatttcaactgccacatttgtcacatttttatggaatctgttagtggattcatctttttgttaatccatgtgtttattatatgtgactatttttgtaaacgaagtttctgttgagaaataggctaaggacctctataacaggtatgccacctggggggtatggcaaccacatggccctcccagctacacaaagtcgtggtttgcatgagggcatgctgcacttagagatcatgcatgagaaaaagtcacaagaaaaacaaattcttaaatttcaccatatttctgggaggggtaattgggtgataagtggaggtgctttgttgatcttgttttgcgaaatccagcccctagaccaagtagattatttgtgggtaggccagtaaatcttagcaggtgcaaacttcattcaaatgtttggagtcataaatgttatgtttctttttgttgtattaaaaaaaaaacctgaatagtgaatattgcccctcaccctccaccgccagaagactgaattgaccaaaattactctttataaatttctgctttttcctgcactttgtttagccatcttcggctctcagcaaggttgacactgtatatgttaatgaaatgctatttattatgtaaatagtcattttaccctgtggtgcacgtttgagcaaacaaataatgacctaagcacagtatttattgcatcaaatatgtaccacaagaaatgtagagtgcaagctttacacaggtaataaaatgtattctgtaccatttatagatagtttggatgctatcaatgcatgtttatattaccatgctgctgtatctggtttctctcactgctcagaatctcatttatgagaaaccatatgtcagtggtaaagtcaaggaaattgttcaacagatctcatttatttaagtcattaagcaatagtttgcagcactttaacagctttttggttatttttacattttaagtggataacatatggtatatagccagactgtacagacatgtttaaaaaaacacactgcttaacctattaaatatgtgtttagaattttataagcaaatataaatactgtaaaaagtcactttattttatttttcagcattatgtacataaatatgaagaggaaattatcttcaggttgatatcacaatcacttttcttactttctgtccatagtactttttcatgaaagaaatttgctaaataagacatgaaaacaagactgggtagttgtagatttctgctttttaaattacatttgctaattttagattatttcacaattttaaggagcaaaataggttcacgattcatatccaaattatgctttgcaattggaaaagggtttaaaattttatttatatttctggtagtacctgcactaactgaattgaaggtagtgcttatgttatttttgttctttttttctgacttcggtttatgttttcatttctttggagtaatgctgctctagattgttctaaatagaatgtgggcttcataatttttttttccacaaaaacagagtagtcaacttatatagtcaattacatcaggacattttgtgtttcttacagaagcaaaccataggctcctcttttccttaaaactacttagataaactgtattcgtgaactgcatgctggaaaatgctactattatgctaaataatgctaaccaacatttaaaatgtgcaaaactaataaagattacattttttatttta

SEQ ID NO: 18 - DNA, 8810 bp, Homo sapiens
  misc_feature (1)..(8810): Homo sapienssodium channel, voltage-gated, type II, alpha subunit (SCN2A), transcript variant 2
atgctgttttctaacagacattgggtaccatcgaatgactgtcagaacagaaagctaaggcaaaggagggaggatgctgtggtcatcctttcttgtttttttcttctttaatgaggatagagcacatgtgagattttactttctactccagtaaaaattctgaagaattgcattggagactgttatattcaacacatacgtggattctgtgttatgatttacatttttctttatttcagcactttcttatgcaaggagctaaacagtgattaaaggagcaggatgaaaagatggcacagtcagtgctggtaccgccaggacctgacagcttccgcttctttaccagggaatcccttgctgctattgaacaacgcattgcagaagagaaagctaagagacccaaacaggaacgcaaggatgaggatgatgaaaatggcccaaagccaaacagtgacttggaagcaggaaaatctcttccatttatttatggagacattcctccagagatggtgtcagtgcccctggaggatctggacccctactatatcaataagaaaacgtttatagtattgaataaagggaaagcaatctctcgattcagtgccacccctgccctttacattttaactcccttcaaccctattagaaaattagctattaagattttggtacattctttattcaatatgctcattatgtgcacgattcttaccaactgtgtatttatgaccatgagtaaccctccagactggacaaagaatgtggagtatacctttacaggaatttatacttttgaatcacttattaaaatacttgcaaggggcttttgtttagaagatttcacatttttacgggatccatggaattggttggatttcacagtcattacttttgcatatgtgacagagtttgtggacctgggcaatgtctcagcgttgagaacattcagagttctccgagcattgaaaacaatttcagtcattccaggcctgaagaccattgtgggggccctgatccagtcagtgaagaagctttctgatgtcatgatcttgactgtgttctgtctaagcgtgtttgcgctaataggattgcagttgttcatgggcaacctacgaaataaatgtttgcaatggcctccagataattcttcctttgaaataaatatcacttccttctttaacaattcattggatgggaatggtactactttcaataggacagtgagcatatttaactgggatgaatatattgaggataaaagtcacttttattttttagaggggcaaaatgatgctctgctttgtggcaacagctcagatgcaggccagtgtcctgaaggatacatctgtgtgaaggctggtagaaaccccaactatggctacacgagctttgacacctttagttgggcctttttgtccttatttcgtctcatgactcaagacttctgggaaaacctttatcaactgacactacgtgctgctgggaaaacgtacatgatattttttgtgctggtcattttcttgggctcattctatctaataaatttgatcttggctgtggtggccatggcctatgaggaacagaatcaggccacattggaagaggctgaacagaaggaagctgaatttcagcagatgctcgaacagttgaaaaagcaacaagaagaagctcaggcggcagctgcagccgcatctgctgaatcaagagacttcagtggtgctggtgggataggagttttttcagagagttcttcagtagcatctaagttgagctccaaaagtgaaaaagagctgaaaaacagaagaaagaaaaagaaacagaaagaacagtctggagaagaagagaaaaatgacagagtccgaaaatcggaatctgaagacagcataagaagaaaaggtttccgtttttccttggaaggaagtaggctgacatatgaaaagagattttcttctccacaccagtccttactgagcatccgtggctcccttttctctccaagacgcaacagtagggcgagccttttcagcttcagaggtcgagcaaaggacattggctctgagaatgactttgctgatgatgagcacagcacctttgaggacaatgacagccgaagagactctctgttcgtgccgcacagacatggagaacggcgccacagcaatgtcagccaggccagccgtgcctccagggtgctccccatcctgcccatgaatgggaagatgcatagcgctgtggactgcaatggtgtggtctccctggtcgggggcccttctaccctcacatctgctgggcagctcctaccagagggcacaactactgaaacagaaataagaaagagacggtccagttcttatcatgtttccatggatttattggaagatcctacatcaaggcaaagagcaatgagtatagccagtattttgaccaacaccatggaagaacttgaagaatccagacagaaatgcccaccatgctggtataaatttgctaatatgtgtttgatttgggactgttgtaaaccatggttaaaggtgaaacaccttgtcaacctggttgtaatggacccatttgttgacctggccatcaccatctgcattgtcttaaatacactcttcatggctatggagcactatcccatgacggagcagttcagcagtgtactgtctgttggaaacctggtcttcacagggatcttcacagcagaaatgtttctcaagataattgccatggatccatattattactttcaagaaggctggaatatttttgatggttttattgtgagccttagtttaatggaacttggtttggcaaatgtggaaggattgtcagttctccgatcattccggctgctccgagttttcaagttggcaaaatcttggccaactctaaatatgctaattaagatcattggcaattctgtgggggctctaggaaacctcaccttggtattggccatcatcgtcttcatttttgctgtggtcggcatgcagctctttggtaagagctacaaagaatgtgtctgcaagatttccaatgattgtgaactcccacgctggcacatgcatgactttttccactccttcctgatcgtgttccgcgtgctgtgtggagagtggatagagaccatgtgggactgtatggaggtcgctggccaaaccatgtgccttactgtcttcatgatggtcatggtgattggaaatctagtggttctgaacctcttcttggccttgcttttgagttccttcagttctgacaatcttgctgccactgatgatgataacgaaatgaataatctccagattgctgtgggaaggatgcagaaaggaatcgattttgttaaaagaaaaatacgtgaatttattcagaaagcctttgttaggaagcagaaagctttagatgaaattaaaccgcttgaagatctaaataataaaaaagacagctgtatttccaaccataccaccatagaaataggcaaagacctcaattatctcaaagacggaaatggaactactagtggcataggcagcagtgtagaaaaatatgtcgtggatgaaagtgattacatgtcatttataaacaaccctagcctcactgtgacagtaccaattgctgttggagaatctgactttgaaaatttaaatactgaagaattcagcagcgagtcagatatggaggaaagcaaagagaagctaaatgcaactagttcatctgaaggcagcacggttgatattggagctcccgccgagggagaacagcctgaggttgaacctgaggaatcccttgaacctgaagcctgttttacagaagactgtgtacggaagttcaagtgttgtcagataagcatagaagaaggcaaagggaaactctggtggaatttgaggaaaacatgctataagatagtggagcacaattggttcgaaaccttcattgtcttcatgattctgctgagcagtggggctctggcctttgaagatatatacattgagcagcgaaaaaccattaagaccatgttagaatatgctgacaaggttttcacttacatattcattctggaaatgctgctaaagtgggttgcatatggttttcaagtgtattttaccaatgcctggtgctggctagacttcctgattgttgatgtctcactggttagcttaactgcaaatgccttgggttactcagaacttggtgccatcaaatccctcagaacactaagagctctgaggccactgagagctttgtcccggtttgaaggaatgagggttgttgtaaatgctcttttaggagccattccatctatcatgaatgtacttctggtttgtctgatcttttggctaatattcagtatcatgggagtgaatctctttgctggcaagttttaccattgtattaattacaccactggagagatgtttgatgtaagcgtggtcaacaactacagtgagtgcaaagctctcattgagagcaatcaaactgccaggtggaaaaatgtgaaagtaaactttgataacgtaggacttggatatctgtctctacttcaagtagccacgtttaagggatggatggatattatgtatgcagctgttgattcacgaaatgtagaattacaacccaagtatgaagacaacctgtacatgtatctttattttgtcatctttattatttttggttcattctttaccttgaatcttttcattggtgtcatcatagataacttcaaccaacagaaaaagaagtttggaggtcaagacatttttatgacagaagaacagaagaaatactacaatgcaatgaaaaaactgggttcaaagaaaccacaaaaacccatacctcgacctgctaacaaattccaaggaatggtctttgattttgtaaccaaacaagtctttgatatcagcatcatgatcctcatctgccttaacatggtcaccatgatggtggaaaccgatgaccagagtcaagaaatgacaaacattctgtactggattaatctggtgtttattgttctgttcactggagaatgtgtgctgaaactgatctctcttcgttactactatttcactattggatggaatatttttgattttgtggtggtcattctctccattgtaggaatgtttctggctgaactgatagaaaagtattttgtgtcccctaccctgttccgagtgatccgtcttgccaggattggccgaatcctacgtctgatcaaaggagcaaaggggatccgcacgctgctctttgctttgatgatgtcccttcctgcgttgtttaacatcggcctccttcttttcctggtcatgttcatctacgccatctttgggatgtccaattttgcctatgttaagagggaagttgggatcgatgacatgttcaactttgagacctttggcaacagcatgatctgcctgttccaaattacaacctctgctggctgggatggattgctagcacctattcttaatagtggacctccagactgtgaccctgacaaagatcaccctggaagctcagttaaaggagactgtgggaacccatctgttgggattttcttttttgtcagttacatcatcatatccttcctggttgtggtgaacatgtacatcgcggtcatcctggagaacttcagtgttgctactgaagaaagtgcagagcctctgagtgaggatgactttgagatgttctatgaggtttgggagaagtttgatcccgatgcgacccagtttatagagtttgccaaactttctgattttgcagatgccctggatcctcctcttctcatagcaaaacccaacaaagtccagctcattgccatggatctgcccatggtgagtggtgaccggatccactgtcttgacatcttatttgcttttacaaagcgtgttttgggtgagagtggagagatggatgcccttcgaatacagatggaagagcgattcatggcatcaaacccctccaaagtctcttatgagcccattacgaccacgttgaaacgcaaacaagaggaggtgtctgctattattatccagagggcttacagacgctacctcttgaagcaaaaagttaaaaaggtatcaagtatatacaagaaagacaaaggcaaagaatgtgatggaacacccatcaaagaagatactctcattgataaactgaatgagaattcaactccagagaaaaccgatatgacgccttccaccacgtctccaccctcgtatgatagtgtgaccaaaccagaaaaagaaaaatttgaaaaagacaaatcagaaaaggaagacaaagggaaagatatcagggaaagtaaaaagtaaaaagaaaccaagaattttccattttgtgatcaattgtttacagcccgtgatggtgatgtgtttgtgtcaacaggactcccacaggaggtctatgccaaactgactgtttttacaaatgtatacttaaggtcagtgcctataacaagacagagacctctggtcagcaaactggaactcagtaaactggagaaatagtatcgatgggaggtttctattttcacaaccagctgacactgctgaagagcagaggcgtaatggctactcagacgataggaaccaatttaaaggggggagggaagttaaatttttatgtaaattcaacatgtgacacttgataatagtaattgtcaccagtgtttatgttttaactgccacacctgccatatttttacaaaacgtgtgctgtgaatttatcacttttctttttaattcacaggttgtttactattatatgtgactatttttgtaaatgggtttgtgtttggggagagggattaaagggagggaattctacatttctctattgtattgtataactggatatattttaaatggaggcatgctgcaattctcattcacacataaaaaaatcacatcacaaaagggaagagtttacttcttgtttcaggatgtttttagatttttgaggtgcttaaatagctattcgtatttttaaggtgtctcatccagaaaaaatttaatgtgcctgtaaatgttccatagaatcacaagcattaaagagttgttttatttttacataacccattaaatgtacatgtatatatgtatatatgtatatgtgcgtgtatatacatatatatgtatacacacatgcacacacagagatatacacataccattacattgtcattcacagtcccagcagcatgactatcacatttttgataagtgtcctttggcataaaataaaaatatcctatcagtcctttctaagaagcctgaattgaccaaaaaacatccccaccaccactttataaagttgattctgctttatcctgcagtattgtttagccatcttctgctcttggtaaggttgacatagtatatgtcaatttaaaaaataaaagtctgctttgtaaatagtaattttacccagtggtgcatgtttgagcaaacaaaaatgatgatttaagcacactacttattgcatcaaatatgtaccacagtaagtatagtttgcaagctttcaacaggtaatatgatgtaattggttccattatagtttgaagctgtcactgctgcatgtttatcttgcctatgctgctgtatcttattccttccactgttcagaagtctaatatgggaagccatatatcagtggtaaagtgaagcaaattgttctaccaagacctcattcttcatgtcattaagcaataggttgcagcaaacaaggaagagcttcttgctttttattcttccaaccttaattgaacactcaatgatgaaaagcccgactgtacaaacatgttgcaagctgcttaaatctgtttaaaatatatggttagagttttctaagaaaatataaatactgtaaaaagttcattttattttatttttcagccttttgtacgtaaaatgagaaattaaaagtatcttcaggtggatgtcacagtcactattgttagtttctgttcctagcacttttaaattgaagcacttcacaaaataagaagcaaggactaggatgcagtgtaggtttctgcttttttattagtactgtaaacttgcacacatttcaatgtgaaacaaatctcaaactgagttcaatgtttatttgctttcaatagtaatgccttatcattgaaagaggcttaaagaaaaaaaaaatcagctgatactcttggcattgcttgaatccaatgtttccacctagtctttttattcagtaatcatcagtcttttccaatgtttgtttacacagatagatcttattgacccatatggcactagaactgtatcagatataatatgggatcccagctttttttcctctcccacaaaaccaggtagtgaagttatattaccagttacagcaaaatactttgtgtttcacaagcaacaataaatgtagattctttatactgaagctattgacttgtagtgtgttggtgaaatgcatgcaggaaaatgctgttaccataaagaacggtaaaccacattacaatcaagccaaaagaataaaggtttcgcttttgtttttgtatttaattgttgtctttgtttctatctttgaaatgccatttaaaggtagatttctatcatgtaaaaataatctatctgaaaaacaaatgtaaagaacacacattaattactataattcatctttcaattttttcatggaatggaagttaattaagaagagtgtattggataactactttaatattggccaaaaagctagatatggcatcaggtagactagtggaaagttacaaaaattaataaaaaattgactaacattttaaaaaaaaaaaaaaaaaa

SEQ ID NO: 19 - DNA, 8994 bp, Homo sapiens
  misc_feature (1)..(8994): Homo sapienssodium channel, voltage-gated, type III, alpha subunit (SCN3A), transcript variant 2
accatagagtgaatctcagaacaggaagcggaggcataagcagagaggattctggaaaggtctctttgttttcttatccacagagaaagaaagaaaaaaaattgtaactaatttgtaaacctctgtggtcaaaaaaaaaaaaaaaaaaaaaagctgaacagctgcagaggaagacacgttataccctaaccatcttggatgctgggctttgttatgctgtaattcataaggctctgttttatcagagattatggagcaagaaaactgaagccaagccacatcaaggtttgacagggatgagatacctgtcaaggattcatagtagagtggcttactgggaaaggagcaaagaatctcttctagggatattgtaagaataaatgagataattcacagaagggacctggagcttttccggaaaaaggtgctgtgactatctaaggtaattcgtatgcaagaagctacacgtaattaaatgtgcaggatgaaaagatggcacaggcactgttggtacccccaggacctgaaagcttccgcctttttactagagaatctcttgctgctatcgaaaaacgtgctgcagaagagaaagccaagaagcccaaaaaggaacaagataatgatgatgagaacaaaccaaagccaaatagtgacttggaagctggaaagaaccttccatttatttatggagacattcctccagagatggtgtcagagcccctggaggacctggatccctactatatcaataagaaaacttttatagtaatgaataaaggaaaggcaattttccgattcagtgccacctctgccttgtatattttaactccactaaaccctgttaggaaaattgctatcaagattttggtacattctttattcagcatgcttatcatgtgcactattttgaccaactgtgtatttatgaccttgagcaaccctcctgactggacaaagaatgtagagtacacattcactggaatctatacctttgagtcacttataaaaatcttggcaagagggttttgcttagaagattttacgtttcttcgtgatccatggaactggctggatttcagtgtcattgtgatggcatatgtgacagagtttgtggacctgggcaatgtctcagcgttgagaacattcagagttctccgagcactgaaaacaatttcagtcattccaggtttaaagaccattgtgggggccctgatccagtcggtaaagaagctttctgatgtgatgatcctgactgtgttctgtctgagcgtgtttgctctcattgggctgcagctgttcatgggcaatctgaggaataaatgtttgcagtggcccccaagcgattctgcttttgaaaccaacaccacttcctactttaatggcacaatggattcaaatgggacatttgttaatgtaacaatgagcacatttaactggaaggattacattggagatgacagtcacttttatgttttggatgggcaaaaagaccctttactctgtggaaatggctcagatgcaggccagtgtccagaaggatacatctgtgtgaaggctggtcgaaaccccaactatggctacacaagctttgacacctttagctgggctttcctgtctctatttcgactcatgactcaagactactgggaaaatctttaccagttgacattacgtgctgctgggaaaacatacatgatattttttgtcctggtcattttcttgggctcattttatttggtgaatttgatcctggctgtggtggccatggcctatgaggagcagaatcaggccaccttggaagaagcagaacaaaaagaggccgaatttcagcagatgctcgaacagcttaaaaagcaacaggaagaagctcaggcagttgcggcagcatcagctgcttcaagagatttcagtggaataggtgggttaggagagctgttggaaagttcttcagaagcatcaaagttgagttccaaaagtgctaaagaatggaggaaccgaaggaagaaaagaagacagagagagcaccttgaaggaaacaacaaaggagagagagacagctttcccaaatccgaatctgaagacagcgtcaaaagaagcagcttccttttctccatggatggaaacagactgaccagtgacaaaaaattctgctcccctcatcagtctctcttgagtatccgtggctccctgttttccccaagacgcaatagcaaaacaagcattttcagtttcagaggtcgggcaaaggatgttggatctgaaaatgactttgctgatgatgaacacagcacatttgaagacagcgaaagcaggagagactcactgtttgtgccgcacagacatggagagcgacgcaacagtaacggcaccaccacagaaacggaagtcagaaagagaaggttaagctcttaccagatttcaatggagatgctggaggattcctctggaaggcaaagagccgtgagcatagccagcattctgaccaacacaatggaagaacttgaagaatctagacagaaatgtccgccatgctggtatagatttgccaatgtgttcttgatctgggactgctgtgatgcatggttaaaagtaaaacatcttgtgaatttaattgttatggatccatttgttgatcttgccatcactatttgcattgtcttaaataccctctttatggccatggagcactaccccatgactgagcaattcagtagtgtgttgactgtaggaaacctggtctttactgggattttcacagcagaaatggttctcaagatcattgccatggatccttattactatttccaagaaggctggaatatctttgatggaattattgtcagcctcagtttaatggagcttggtctgtcaaatgtggagggattgtctgtactgcgatcattcagactgcttagagttttcaagttggcaaaatcctggcccacactaaatatgctaattaagatcattggcaattctgtgggggctctaggaaacctcaccttggtgttggccatcatcgtcttcatttttgctgtggtcggcatgcagctctttggtaagagctacaaagaatgtgtctgcaagatcaatgatgactgtacgctcccacggtggcacatgaacgacttcttccactccttcctgattgtgttccgcgtgctgtgtggagagtggatagagaccatgtgggactgtatggaggtcgctggccaaaccatgtgccttattgttttcatgttggtcatggtcattggaaaccttgtggttctgaacctctttctggccttattgttgagttcatttagctcagacaaccttgctgctactgatgatgacaatgaaatgaataatctgcagattgcagtaggaagaatgcaaaagggaattgattatgtgaaaaataagatgcgggagtgtttccaaaaagccttttttagaaagccaaaagttatagaaatccatgaaggcaataagatagacagctgcatgtccaataatactggaattgaaataagcaaagagcttaattatcttagagatgggaatggaaccaccagtggtgtaggtactggaagcagtgttgaaaaatacgtaatcgatgaaaatgattatatgtcattcataaacaaccccagcctcaccgtcacagtgccaattgctgttggagagtctgactttgaaaacttaaatactgaagagttcagcagtgagtcagaactagaagaaagcaaagagaaattaaatgcaaccagctcatctgaaggaagcacagttgatgttgttctaccccgagaaggtgaacaagctgaaactgaacccgaagaagaccttaaaccggaagcttgttttactgaaggatgtattaaaaagtttccattctgtcaagtaagtacagaagaaggcaaagggaagatctggtggaatcttcgaaaaacctgctacagtattgttgagcacaactggtttgagactttcattgtgttcatgatccttctcagtagtggtgcattggcctttgaagatatatacattgaacagcgaaagactatcaaaaccatgctagaatatgctgacaaagtctttacctatatattcattctggaaatgcttctcaaatgggttgcttatggatttcaaacatatttcactaatgcctggtgctggctagatttcttgatcgttgatgtttctttggttagcctggtagccaatgctcttggctactcagaactcggtgccatcaaatcattacggacattaagagctttaagacctctaagagccttatcccggtttgaaggcatgagggtggttgtgaatgctcttgttggagcaattccctctatcatgaatgtgctgttggtctgtctcatcttctggttgatctttagcatcatgggtgtgaatttgtttgctggcaagttctaccactgtgttaacatgacaacgggtaacatgtttgacattagtgatgttaacaatttgagtgactgtcaggctcttggcaagcaagctcggtggaaaaacgtgaaagtaaactttgataatgttggcgctggctatcttgcactgcttcaagtggccacatttaaaggctggatggatattatgtatgcagctgttgattcacgagatgttaaacttcagcctgtatatgaagaaaatctgtacatgtatttatactttgtcatctttatcatctttgggtcattcttcactctgaatctattcattggtgtcatcatagataacttcaaccagcagaaaaagaagtttggaggtcaagacatctttatgacagaggaacagaaaaaatattacaatgcaatgaagaaacttggatccaagaaacctcagaaacccatacctcgcccagcaaacaaattccaaggaatggtctttgattttgtaaccagacaagtctttgatatcagcatcatgatcctcatctgcctcaacatggtcaccatgatggtggaaacggatgaccagggcaaatacatgaccctagttttgtcccggatcaacctagtgttcattgttctgttcactggagaatttgtgctgaagctcgtctccctcagacactactacttcactataggctggaacatctttgactttgtggtggtgattctctccattgtaggtatgtttctggctgagatgatagaaaagtattttgtgtcccctaccttgttccgagtgatccgtcttgccaggattggccgaatcctacgtctgatcaaaggagcaaaggggatccgcacgctgctctttgctttgatgatgtcccttcctgcgttgtttaacatcggcctcctgctcttcctggtcatgtttatctatgccatctttgggatgtccaactttgcctatgttaaaaaggaagctggaattgatgacatgttcaactttgagacctttggcaacagcatgatctgcttgttccaaattacaacctctgctggctgggatggattgctagcacctattcttaatagtgcaccacccgactgtgaccctgacacaattcaccctggcagctcagttaagggagactgtgggaacccatctgttgggattttcttttttgtcagttacatcatcatatccttcctggttgtggtgaacatgtacatcgcggtcatcctggagaacttcagtgttgctactgaagaaagtgcagagcccctgagtgaggatgactttgagatgttctatgaggtttgggaaaagtttgatcccgatgcgacccagtttatagagttctctaaactctctgattttgcagctgccctggatcctcctcttctcatagcaaaacccaacaaagtccagcttattgccatggatctgcccatggtcagtggtgaccggatccactgtcttgatattttatttgcctttacaaagcgtgttttgggtgagagtggagagatggatgcccttcgaatacagatggaagacaggtttatggcatcaaacccctccaaagtctcttatgagcctattacaaccactttgaaacgtaaacaagaggaggtgtctgccgctatcattcagcgtaatttcagatgttatcttttaaagcaaaggttaaaaaatatatcaagtaactataacaaagaggcaattaaagggaggattgacttacctataaaacaagacatgattattgacaaactaaatgggaactccactccagaaaaaacagatgggagttcctctaccacctctcctccttcctatgatagtgtaacaaaaccagacaaggaaaagtttgagaaagacaaaccagaaaaagaaagcaaaggaaaagaggtcagagaaaatcaaaagtaaaaagaaacaaagaattatctttgtgatcaattgtttacagcctatgaaggtaaagtatatgtgtcaactggacttcaagaggaggtccatgccaaactgactgttttaacaaatactcatagtcagtgcctatacaagacagtgaagtgacctctctgtcactgcaactctgtgaagcagggtatcaacattgacaagaggttgctgtttttattaccagctgacactgctgaggagaaacccaatggctacctagactatagggatagttgtgcaaagtgaacattgtaactacaccaaacacctttagtacagtccttgcatccattctatttttaacttccatatctgccatatttttacaaaatttgttctagtgcatttccatggtccccaattcatagtttattcataatgctatgtcactatttttgtaaatgaggtttacgttgaagaaacagtatacaagaaccctgtctctcaaatgatcagacaaaggtgttttgccagagagataaaatttttgctcaaaaccagaaaaagaattgtaatggctacagtttcagttacttccattttctagatggctttaattttgaaagtattttagtctgttatgtttgtttctatctgaacagttatgtgcctgtaaagtctcctctaatatttaaaggattatttttatgcaaagtattctgtttcagcaagtgcaaattttattctaagtttcagagctctatatttaatttaggtcaaatgctttccaaaaagtaatctaataaatccattctagaaaaatatatctaaagtattgctttagaatagttgttccactttctgctgcagtattgctttgccatcttctgctctcagcaaagctgatagtctatgtcaattaaataccctatgttatgtaaatagttattttatcctgtggtgcatgtttgggcaaatatatatatagcctgataaacaacttctattaaatcaaatatgtaccacagtgtatgtgtcttttgcaagcttccaacagggatgtatcctgtatcattcattaaacatagtttaaaggctatcactaatgcatgttaatattgcctatgctgctctattttactcaatccattcttcacaagtcttggttaaagaatgtcacatattggtgatagaatgaattcaacctgctctgtccattatgtcaagcagaataatttgaagctatttacaaacacctttacttttgcacttttaattcaacatgagtatcatatggtatctctctagatttcaaggaaacacactggatactgcctactgacaaaacctattcttcatattttgctaaaaatatgtctaaaacttgtttaaatataaataatgtaaaaatataatcaactttatttgtcagcattttgtacataagaaaattattttcaggttgatgacatcacaatttattttactttatgcttttgcttttgatttttaatcacaattccaaacttttgaatccataagatttttcaatggataatttcctaaaataaaagttagataatgggttttatggatttctttgttataatatattttctaccattccaataggagatacattggtcaaacactcaaacctagatcattttctaccaactatggttgcctcaatataaccttttattcatagatgtttttttttattcaacttttgtagtatttacgtatgcagactagtcttatttttttaattcctgctgcactaaagctattacaaatataacatggactttgttctttttagccatgaacaaagtggcaaagttgtgcaattacctaacatgatataaatttttgttttttgcacaaaccaaaagtttaatgttaattctttttacaaaactatttactgtagtgtattgaagaactgcatgcagggaattgctattgctaaaaagaatggtgagctacgtcattattgagccaaaagaataaatttcattttttattgcatttcacttattggcctctggggttttttgtttttgttttttgctgttggcagtttaaaatatatataattaataaaacctgtgcttgatctgacatttgtatacataaaagtttacatgaattttacaacaaactagtgcatgattcaccaagcagtactacagaacaaaggcaaattaaaagcagctttgtgaacttttatgtgtgcaaaggatcaagttcacatgttccaactttcaggtttgataataatagtagtaaccacctacaatagctttcaatttcaattaactcccttggctataagcatctaaactcatcttctttcaatataattgatgctatctcctaattacttggtggctaataaatgttacattctttgttacttaaatgcattatataaactcctatgtatacataaggtattaatgatatagttattgagaatttatattaacttttttttcaagaacccttggatttatgtgaggtcaaaaccaaactcttattctcagtggaaaactccagttgtaatgcatatttttaaagacaatttggatctaaatatgtatttcataattctcccataataaattatataaggtggctaattggaaaaaaaaaaaaaaaaaa

SEQ ID NO: 20 - DNA, 9771 bp, Homo sapiens
  misc_feature (1)..(9771): Homo sapienssodium channel, voltage-gated, type IX, alpha subunit (SCN9A)
cggggctgctacctccacgggcgcgccctggcaggaggggcgcagtctgcttgcaggcggtcgccagcgctccagcggcggctgtcggctttccaattccgccagctcggctgaggctgggctagcctgggtgccagtggctgctagcggcaggcgtcccctgagcaacaggagcccagagaaaaagaagcagccctgagagagcgccggggaaggagaggcccgcgccctctcctggagccagattctgcaggtgcactgggtggggatgatcggcgggctaggttgcaagcctcttatgtgaggagctgaagaggaattaaaatatacaggatgaaaagatggcaatgttgcctcccccaggacctcagagctttgtccatttcacaaaacagtctcttgccctcattgaacaacgcattgctgaaagaaaatcaaaggaacccaaagaagaaaagaaagatgatgatgaagaagccccaaagccaagcagtgacttggaagctggcaaacagctgcccttcatctatggggacattcctcccggcatggtgtcagagcccctggaggacttggacccctactatgcagacaaaaagactttcatagtattgaacaaagggaaaacaatcttccgtttcaatgccacacctgctttatatatgctttctcctttcagtcctctaagaagaatatctattaagattttagtacactccttattcagcatgctcatcatgtgcactattctgacaaactgcatatttatgaccatgaataacccaccggactggaccaaaaatgtcgagtacacttttactggaatatatacttttgaatcacttgtaaaaatccttgcaagaggcttctgtgtaggagaattcacttttcttcgtgacccgtggaactggctggattttgtcgtcattgtttttgcgtatttaacagaatttgtaaacctaggcaatgtttcagctcttcgaactttcagagtattgagagctttgaaaactatttctgtaatcccaggcctgaagacaattgtaggggctttgatccagtcagtgaagaagctttctgatgtcatgatcctgactgtgttctgtctgagtgtgtttgcactaattggactacagctgttcatgggaaacctgaagcataaatgttttcgaaattcacttgaaaataatgaaacattagaaagcataatgaataccctagagagtgaagaagactttagaaaatatttttattacttggaaggatccaaagatgctctcctttgtggtttcagcacagattcaggtcagtgtccagaggggtacacctgtgtgaaaattggcagaaaccctgattatggctacacgagctttgacactttcagctgggccttcttagccttgtttaggctaatgacccaagattactgggaaaacctttaccaacagacgctgcgtgctgctggcaaaacctacatgatcttctttgtcgtagtgattttcctgggctccttttatctaataaacttgatcctggctgtggttgccatggcatatgaagaacagaaccaggcaaacattgaagaagctaaacagaaagaattagaatttcaacagatgttagaccgtcttaaaaaagagcaagaagaagctgaggcaattgcagcggcagcggctgaatatacaagtattaggagaagcagaattatgggcctctcagagagttcttctgaaacatccaaactgagctctaaaagtgctaaagaaagaagaaacagaagaaagaaaaagaatcaaaagaagctctccagtggagaggaaaagggagatgctgagaaattgtcgaaatcagaatcagaggacagcatcagaagaaaaagtttccaccttggtgtcgaagggcataggcgagcacatgaaaagaggttgtctacccccaatcagtcaccactcagcattcgtggctccttgttttctgcaaggcgaagcagcagaacaagtctttttagtttcaaaggcagaggaagagatataggatctgagactgaatttgccgatgatgagcacagcatttttggagacaatgagagcagaaggggctcactgtttgtgccccacagaccccaggagcgacgcagcagtaacatcagccaagccagtaggtccccaccaatgctgccggtgaacgggaaaatgcacagtgctgtggactgcaacggtgtggtctccctggttgatggacgctcagccctcatgctccccaatggacagcttctgccagagggcacgaccaatcaaatacacaagaaaaggcgttgtagttcctatctcctttcagaggatatgctgaatgatcccaacctcagacagagagcaatgagtagagcaagcatattaacaaacactgtggaagaacttgaagagtccagacaaaaatgtccaccttggtggtacagatttgcacacaaattcttgatctggaattgctctccatattggataaaattcaaaaagtgtatctattttattgtaatggatccttttgtagatcttgcaattaccatttgcatagttttaaacacattatttatggctatggaacaccacccaatgactgaggaattcaaaaatgtacttgctataggaaatttggtctttactggaatctttgcagctgaaatggtattaaaactgattgccatggatccatatgagtatttccaagtaggctggaatatttttgacagccttattgtgactttaagtttagtggagctctttctagcagatgtggaaggattgtcagttctgcgatcattcagactgctccgagtcttcaagttggcaaaatcctggccaacattgaacatgctgattaagatcattggtaactcagtaggggctctaggtaacctcaccttagtgttggccatcatcgtcttcatttttgctgtggtcggcatgcagctctttggtaagagctacaaagaatgtgtctgcaagatcaatgatgactgtacgctcccacggtggcacatgaacgacttcttccactccttcctgattgtgttccgcgtgctgtgtggagagtggatagagaccatgtgggactgtatggaggtcgctggtcaagctatgtgccttattgtttacatgatggtcatggtcattggaaacctggtggtcctaaacctatttctggccttattattgagctcatttagttcagacaatcttacagcaattgaagaagaccctgatgcaaacaacctccagattgcagtgactagaattaaaaagggaataaattatgtgaaacaaaccttacgtgaatttattctaaaagcattttccaaaaagccaaagatttccagggagataagacaagcagaagatctgaatactaagaaggaaaactatatttctaaccatacacttgctgaaatgagcaaaggtcacaatttcctcaaggaaaaagataaaatcagtggttttggaagcagcgtggacaaacacttgatggaagacagtgatggtcaatcatttattcacaatcccagcctcacagtgacagtgccaattgcacctggggaatccgatttggaaaatatgaatgctgaggaacttagcagtgattcggatagtgaatacagcaaagtgagattaaaccggtcaagctcctcagagtgcagcacagttgataaccctttgcctggagaaggagaagaagcagaggctgaacctatgaattccgatgagccagaggcctgtttcacagatggttgtgtacggaggttctcatgctgccaagttaacatagagtcagggaaaggaaaaatctggtggaacatcaggaaaacctgctacaagattgttgaacacagttggtttgaaagcttcattgtcctcatgatcctgctcagcagtggtgccctggcttttgaagatatttatattgaaaggaaaaagaccattaagattatcctggagtatgcagacaagatcttcacttacatcttcattctggaaatgcttctaaaatggatagcatatggttataaaacatatttcaccaatgcctggtgttggctggatttcctaattgttgatgtttctttggttactttagtggcaaacactcttggctactcagatcttggccccattaaatcccttcggacactgagagctttaagacctctaagagccttatctagatttgaaggaatgagggtcgttgtgaatgcactcataggagcaattccttccatcatgaatgtgctacttgtgtgtcttatattctggctgatattcagcatcatgggagtaaatttgtttgctggcaagttctatgagtgtattaacaccacagatgggtcacggtttcctgcaagtcaagttccaaatcgttccgaatgttttgcccttatgaatgttagtcaaaatgtgcgatggaaaaacctgaaagtgaactttgataatgtcggacttggttacctatctctgcttcaagttgcaacttttaagggatggacgattattatgtatgcagcagtggattctgttaatgtagacaagcagcccaaatatgaatatagcctctacatgtatatttattttgtcgtctttatcatctttgggtcattcttcactttgaacttgttcattggtgtcatcatagataatttcaaccaacagaaaaagaagcttggaggtcaagacatctttatgacagaagaacagaagaaatactataatgcaatgaaaaagctggggtccaagaagccacaaaagccaattcctcgaccagggaacaaaatccaaggatgtatatttgacctagtgacaaatcaagcctttgatattagtatcatggttcttatctgtctcaacatggtaaccatgatggtagaaaaggagggtcaaagtcaacatatgactgaagttttatattggataaatgtggtttttataatccttttcactggagaatgtgtgctaaaactgatctccctcagacactactacttcactgtaggatggaatatttttgattttgtggttgtgattatctccattgtaggtatgtttctagctgatttgattgaaacgtattttgtgtcccctaccctgttccgagtgatccgtcttgccaggattggccgaatcctacgtctagtcaaaggagcaaaggggatccgcacgctgctctttgctttgatgatgtcccttcctgcgttgtttaacatcggcctcctgctcttcctggtcatgttcatctacgccatctttggaatgtccaactttgcctatgttaaaaaggaagatggaattaatgacatgttcaattttgagacctttggcaacagtatgatttgcctgttccaaattacaacctctgctggctgggatggattgctagcacctattcttaacagtaagccacccgactgtgacccaaaaaaagttcatcctggaagttcagttgaaggagactgtggtaacccatctgttggaatattctactttgttagttatatcatcatatccttcctggttgtggtgaacatgtacattgcagtcatactggagaattttagtgttgccactgaagaaagtactgaacctctgagtgaggatgactttgagatgttctatgaggtttgggagaagtttgatcccgatgcgacccagtttatagagttctctaaactctctgattttgcagctgccctggatcctcctcttctcatagcaaaacccaacaaagtccagctcattgccatggatctgcccatggttagtggtgaccggatccattgtcttgacatcttatttgcttttacaaagcgtgttttgggtgagagtggggagatggattctcttcgttcacagatggaagaaaggttcatgtctgcaaatccttccaaagtgtcctatgaacccatcacaaccacactaaaacggaaacaagaggatgtgtctgctactgtcattcagcgtgcttatagacgttaccgcttaaggcaaaatgtcaaaaatatatcaagtatatacataaaagatggagacagagatgatgatttactcaataaaaaagatatggcttttgataatgttaatgagaactcaagtccagaaaaaacagatgccacttcatccaccacctctccaccttcatatgatagtgtaacaaagccagacaaagagaaatatgaacaagacagaacagaaaaggaagacaaagggaaagacagcaaggaaagcaaaaaatagagcttcatttttgatatattgtttacagcctgtgaaagtgatttatttgtgttaataaaactcttttgaggaagtctatgccaaaatcctttttatcaaaatattctcgaaggcagtgcagtcactaactctgatttcctaagaaaggtgggcagcattagcagatggttatttttgcactgatgattctttaagaatcgtaagagaactctgtaggaattattgattatagcatacaaaagtgattcagttttttggtttttaataaatcagaagaccatgtagaaaacttttacatctgccttgtcatcttttcacaggattgtaattagtcttgtttcccatgtaaataaacaacacacgcatacagaaaaatctattatttatctattatttggaaatcaacaaaagtatttgccttggctttgcaatgaaatgcttgatagaagtaatggacattagttatgaatgtttagttaaaatgcattattagggagcttgactttttatcaatgtacagaggttattctatattttgaggtgcttaaatttattctacattgcatcagaaccaatttatatgtgcctataaaatgccatgggattaaaaatatatgtaggctattcatttctacaaatgtttttcattcatcttgactcacatgccaacaaggataagacttacctttagagtattgtgtttcatagcctttcttctttcatatccctttttgttcatagaataaccacagaacttgaaaaattattctaagtacatattacactcctcaaaaaaaacaaagataactgagaaaaaagttattgacagaagttctatttgctattatttacatagcctaacatttgactgtgctgcccaaaatactgataatagtctcttaaactcttttgtcaaattttcctgctttcttatgcagtattgtttagtcatcctttcgctgtaagcaaagttgatgaaatccttcctgatatgcagttagttgtttgaccacggtacatacttgagcagataataacttgggcacagtatttattgcatcacttgtatacaatcccgtgtttggcaagctttcaaatcatgtaatatgacagactttacacagatatgtgtttagtatgaataaaaaagcattgaaatagggattcttgccaacttgctctcttgccaccaacttactttcctaaattatggaagtaatcttttttggatatacttcaatgtatacaatgaggaagatgtcaccttctccttaaaattctatgatgtgaaatatattttgcctcaatcaacacagtaccatgggcttctaatttatcaagcacatattcattttgcattagctgtagacatctagttttttgaaaacacctattaatagtaatttgaaaagaaataaccataatgctttttttcgtgagtttatttcaggaatatgagatctttcttctataaagttattcatgcacaggcaaaaattgagctacacaggtagaatgtagttttacttagaagatttttgtgggaggttttgaagcaaatatataaaacaactttcactaatttgctttccatatttaaaaaataataaattacatttatataataaatgtttaaagcacatattttttgttgttctggcaatttaaaaagaaagaggatttaaacgtacctatagaaacaaagatttatggttaaagaatgagatcagaagtctagaatgtttttaaattgtgatatattttacaacatccgttattactttgagacatttgtcctaatctacgtataaaactcaatctagggctaaagattctttataccatcttaggttcattcatcttaggctatttgaaccactttttaatttaatatgaaagacaccatgcagtgttttccgagactacatagatcattttatcacatacctaccaagcctgttggaaataggttttgataatttaagtagggacctatacaaaatatattacatttatcagatttttaaatacattcaattaagaatttaacatcaccttaaatttgaattcaatctaccgttatttcaaactcacaaatataactgcattatgaatacttacataatgtagtaagacaagatgtttgacaggttcgtgtgtaattttctattaatgtttttacattgccttgtttttatgtaaaataaaaaatatgggcaactggtttgttaacaacacaatttcttcttagcatttcaaaaatatatataaagttgttctttttcctatttcatgaactatgtttttttttaaaataacatggttaagttttatatatatttacgtttgtttcaggaatgtctacttgtgactttttatcaattaaaaataatatttggaagaaagagcttattaagtataagcttgaagtaaaattagacctctctttccatgtagattactgtttgtactgatggtttcacccttcagaaggcactgtcatattaatatttaaattttataatcgctgaacttattacacccaacaatacagaaaggcagttacactgaagaacttaacttagaataaaatggaagcaaacaggttttctaaaaacttttttaagtgaccaggtctcgctctgtcacccaggctagagtgcaatggcatgatcatagctctctgcagcctcaactctgggctcaagcaaccctcctgcctcagcctcccaagtagctaagactacaggtacatgccaccatgcctggctaatatttaaatttttgtagataaggggtcttgctatgttgcccaggctagtctcaaactcctggcttcaagtgttcctactgtcatgacctgccaacatgctggggttacaggcatgagccaccatgccccaaacaggtttgaacacaaatctttcggatgaaaattagagaacctaattttagctttttgatagttacctagtttgcaaaagatttgggtgacttgtgagctgtttttaaatgctgattgttgaacatcacaacccaaaatacttagcatgattttatagagttttgatagctttattaaaaagagtgaaaataaaatgcatatgtaaataaagcagttctaaatagctatttcagagaaatgttaatagaagtgctgaaagaagggccaactaaattaggatggccagggaattggcctgggtttaggacctatgtatgaaggccaccaattttttaaaaatatctgtggtttattatgttattatcttcttgaggaaaacaatcaagaattgcttcatgaaaataaataaatagccatgaatatcataaagctgtttacataggattctttacaaatttcatagatctatgaatgctcaaaatgtttgagtttgccataaattatattgtagttatattgtagttatacttgagactgacacattgtaatataatctaagaataaaagttatacaaaataaaaaaaaaaaaa

SEQ ID NO: 21 - DNA, 6500 bp, Homo sapiens
  misc_feature (1)..(6500): Homo sapienssodium channel, voltage-gated, type XI, alpha subunit (SCN11A), transcript variant 1
tcctgctatacccacagtggtggtcatctcttctgatcttcacagccaatcagctcccaaggcccctgacctcagctcagcttttgtagatccttatgacaccatcctttaagactggaatcctagggcaggctgttttattcccgcctcctgaggcctttctgaggatctgtggcttgtctctgtcctgagggtgaagatggatgacagatgctacccagtaatctttccagatgagcggaatttccgccccttcacttccgactctctggctgcaattgagaagcggattgccatccaaaaggagaaaaagaagtctaaagaccagacaggagaagtaccccagcctcggcctcagcttgacctaaaggcctccaggaagttgcccaagctctatggcgacattcctcgtgagctcataggaaagcctctggaagacttggacccattctaccgaaatcataagacatttatggtgttaaacagaaagaggacaatctaccgcttcagtgccaagcatgccttgttcatttttgggcctttcaattcaatcagaagtttagccattagagtctcagtccattcattgttcagcatgttcattatcggcaccgttatcatcaactgcgtgttcatggctacagggcctgctaaaaacagcaacagtaacaatactgacattgcagagtgtgtcttcactgggatttatatttttgaagctttgattaaaatattggcaagaggtttcattctggatgagttttctttccttcgagatccatggaactggctggactccattgtcattggaatagcgattgtgtcatatattccaggaatcaccatcaaactattgcccctgcgtaccttccgtgtgttcagagctttgaaagcaatttcagtagtttcacgtctgaaggtcatcgtgggggccttgctacgctctgtgaagaagctggtcaacgtgattatcctcaccttcttttgcctcagcatctttgccctggtaggtcagcagctcttcatgggaagtctgaacctgaaatgcatctcgagggactgtaaaaatatcagtaacccggaagcttatgaccattgctttgaaaagaaagaaaattcacctgaattcaaaatgtgtggcatctggatgggtaacagtgcctgttccatacaatatgaatgtaagcacaccaaaattaatcctgactataattatacgaattttgacaactttggctggtcttttcttgccatgttccggctgatgacccaagattcctgggagaagctttatcaacagaccctgcgtactactgggctctactcagtcttcttcttcattgtggtcattttcctgggctccttctacctgattaacttaaccctggctgttgttaccatggcatatgaggagcagaacaagaatgtagctgcagagatagaggccaaggaaaagatgtttcaggaagcccagcagctgttaaaggaggaaaaggaggctctggttgccatgggaattgacagaagttcacttacttcccttgaaacatcatattttaccccaaaaaagagaaagctctttggtaataagaaaaggaagtccttctttttgagagagtctgggaaagaccagcctcctgggtcagattctgatgaagattgccaaaaaaagccacagctcctagagcaaaccaaacgactgtcccagaatctatcactggaccactttgatgagcatggagatcctctccaaaggcagagagcactgagtgctgtcagcatcctcaccatcaccatgaaggaacaagaaaaatcacaagagccttgtctcccttgtggagaaaacctggcatccaagtacctcgtgtggaactgttgcccccagtggctgtgcgttaagaaggtcctgagaactgtgatgactgacccgtttactgagctggccatcaccatctgcatcatcatcaacactgtcttcttggccatggagcatcacaagatggaggccagttttgagaagatgttgaatatagggaatttggttttcactagcatttttatagcagaaatgtgcctaaaaatcattgcgctcgatccctaccactactttcgccgaggctggaacatttttgacagcattgttgctcttctgagttttgcagatgtaatgaactgtgtacttcaaaagagaagctggccattcttgcgttccttcagagtgctcagggtcttcaagttagccaaatcctggccaactttgaacacactaattaagataatcggcaactctgtcggagcccttggaagcctgactgtggtcctggtcattgtgatctttattttctcagtagttggcatgcagctttttggccgtagcttcaattcccaaaagagtccaaaactctgtaacccgacaggcccgacagtctcatgtttacggcactggcacatgggggatttctggcactccttcctagtggtattccgcatcctctgcggggaatggatcgaaaatatgtgggaatgtatgcaagaagcgaatgcatcatcatcattgtgtgttattgtcttcatattgatcacggtgataggaaaacttgtggtgctcaacctcttcattgccttactgctcaattcctttagcaatgaggaaagaaatggaaacttagaaggagaggccaggaaaactaaagtccagttagcactggatcgattccgccgggctttttgttttgtgagacacactcttgagcatttctgtcacaagtggtgcaggaagcaaaacttaccacagcaaaaagaggtggcaggaggctgtgctgcacaaagcaaagacatcattcccctggtcatggagatgaaaaggggctcagagacccaggaggagcttggtatactaacctctgtaccaaagaccctgggcgtcaggcatgattggacttggttggcaccacttgcggaggaggaagatgacgttgaattttctggtgaagataatgcacagcgcatcacacaacctgagcctgaacaacaggcctatgagctccatcaggagaacaagaagcccacgagccagagagttcaaagtgtggaaattgacatgttctctgaagatgagcctcatctgaccatacaggatccccgaaagaagtctgatgttaccagtatactatcagaatgtagcaccattgatcttcaggatggctttggatggttacctgagatggttcccaaaaagcaaccagagagatgtttgcccaaaggctttggttgctgctttccatgctgtagcgtggacaagagaaagcctccctgggtcatttggtggaacctgcggaaaacctgctaccaaatagtgaaacacagctggtttgagagctttattatctttgtgattctgctgagcagtggggcactgatatttgaagatgttcaccttgagaaccaacccaaaatccaagaattactaaattgtactgacattatttttacacatatttttatcctggagatggtactaaaatgggtagccttcggatttggaaagtatttcaccagtgcctggtgctgccttgatttcatcattgtgattgtctctgtgaccaccctcattaacttaatggaattgaagtccttccggactctacgagcactgaggcctcttcgtgcgctgtcccagtttgaaggaatgaaggtggtggtcaatgctctcataggtgccatacctgccattctgaatgttttgcttgtctgcctcattttctggctcgtattttgtattctgggagtatacttcttttctggaaaatttgggaaatgcattaatggaacagactcagttataaattataccatcattacaaataaaagtcaatgtgaaagtggcaatttctcttggatcaaccagaaagtcaactttgacaatgtgggaaatgcttacctcgctctgctgcaagtggcaacatttaagggctggatggatattatatatgcagctgttgattccacagagaaagaacaacagccagagtttgagagcaattcactcggttacatttacttcgtagtctttatcatctttggctcattcttcactctgaatctcttcattggcgttatcattgacaacttcaaccaacagcagaaaaagttaggtggccaagacatttttatgacagaagaacagaagaaatactataatgcaatgaaaaaattaggatccaaaaaacctcaaaaacccattccacggcctctgaacaaatgtcaaggtctcgtgttcgacatagtcacaagccagatctttgacatcatcatcataagtctcattatcctaaacatgattagcatgatggctgaatcatacaaccaacccaaagccatgaaatccatccttgaccatctcaactgggtctttgtggtcatctttacgttagaatgtctcatcaaaatctttgctttgaggcaatactacttcaccaatggctggaatttatttgactgtgtggtcgtgcttctttccattgttagtacaatgatttctaccttggaaaatcaggagcacattcctttccctccgacgctcttcagaattgtccgcttggctcggattggccgaatcctgaggcttgtccgggctgcacgaggaatcaggactctcctctttgctctgatgatgtcgcttccttctctgttcaacattggtcttctactctttctgattatgtttatctatgccattctgggtatgaactggttttccaaagtgaatccagagtctggaatcgatgacatattcaacttcaagacttttgccagcagcatgctctgtctcttccagataagcacatcagcaggttgggattccctgctcagccccatgctgcgatcaaaagaatcatgtaactcttcctcagaaaactgccacctccctggcatagccacatcctactttgtcagttacattatcatctcctttctcattgttgtcaacatgtacattgctgtgattttagagaacttcaatacagccactgaagaaagtgaggaccctttgggtgaagatgactttgacatattttatgaagtgtgggaaaagtttgacccagaagcaacacaatttatcaaatattctgccctttctgactttgctgatgccttgcctgagcctttgcgtgtcgcaaagccaaataaatatcaatttctagtaatggacttgcccatggtgagtgaagatcgcctccactgcatggatattcttttcgccttcaccgctagggtactcggtggctctgatggcctagatagtatgaaagcaatgatggaagagaagttcatggaagccaatcctctcaagaagttgtatgaacccatagtcaccaccaccaagagaaaggaagaggaaagaggtgctgctattattcaaaaggcctttcgaaagtacatgatgaaggtgaccaagggtgaccaaggtgaccaaaatgacttggaaaacgggcctcattcaccactccagactctttgcaatggagacttgtctagctttggggtggccaagggcaaggtccactgtgactgagccctcacctccacgcctacctcatagcttcacagccttgccttcagcctctgagctccaggggtcagcagcttagtgtatcaacagggagtggattcaccaaattagccattccattttcttttctggctaaaataaatgatatttcaatttcattttaaataatacttacagagatataagataaggctacttgacaaccagtggtactattataataaggaagaagacaccaggaaggactgtaaaaggacataccaattttaggattgaaatagttcaggccgggcgcagtggctcatgcctgtaatcccagcactttgagaggccaaggcaggtggatcacgaggtcaagagatcgagaccatcctggccaacatgatgaaactccgtctctactaaaaatacaaaaattagctgggcatggtggcgtgcgcctgtagtcccagctacttgggaggctgaggcaggagaatcgcttaaacctgggagacggaggttgcagtgagccaagatcgtgccactgcactccagcctggtgacagagtgagactctgtttcaaaaaagaaaagaaaagaaacatggttcaaattatatctaaacaaaaaagaataagaaacaaaaaacacattaaaattttaagttgtattttctatgtttctagatacatcatttttgtttgatattttcctgatgcaagtatgtggtttatcacatgtagctcttttgcatgctaaatgaaaattcaaaacttgccaataaatgaatagcttattgcagacattttttaccaacattaattattttgggtttgtttaaaacctagaggcacaatcttgacttgtcaattactaccctttcacaagctaccatctcagatatatatatatatataaattcaataaagctttctgtttgtgttcc

SEQ ID NO: 22 - DNA, 11447 bp, Homo sapiens
  misc_feature (1)..(11447): Homo sapienssodium channel, voltage gated, type VIII, alpha subunit (SCN8A), transcript variant 2
gagcgctccaagatggcgcccaccgcagtcccgcccgccgcatcctcggcgcctttgcagtccggccgcgcctcccgggccccgcgttagggccgccgctgcctccctcgccgccgccgctgccagctgacctgtcctggacgcagcataactaacgaagctgctgcaggatgagaagatggcagcgcggctgcttgcaccaccaggccctgatagtttcaagcctttcacccctgagtcactggcaaacattgagaggcgcattgctgagagcaagctcaagaaaccaccaaaggccgatggcagtcatcgggaggacgatgaggacagcaagcccaagccaaacagcgacctggaagcagggaagagtttgcctttcatctacggggacatcccccaaggcctggttgcagttcccctggaggactttgacccatactatttgacgcagaaaacctttgtagtattaaacagagggaaaactctcttcagatttagtgccacgcctgccttgtacattttaagtccttttaacctgataagaagaatagctattaaaattttgatacattcagtatttagcatgatcattatgtgcactattttgaccaactgtgtattcatgacttttagtaaccctcctgactggtcgaagaatgtggagtacacgttcacagggatttatacatttgaatcactagtgaaaatcattgcaagaggtttctgcatagatggctttacctttttacgggacccatggaactggttagatttcagtgtcatcatgatggcgtatataacagagtttgtaaacctaggcaatgtttcagctctacgcactttcagggtactgagggctttgaaaactatttcggtaatcccaggcctgaagacaattgtgggtgccctgattcagtctgtgaagaaactgtcagatgtgatgatcctgacagtgttctgcctgagtgtttttgccttgatcggactgcagctgttcatggggaaccttcgaaacaagtgtgttgtgtggcccataaacttcaacgagagctatcttgaaaatggcaccaaaggctttgattgggaagagtatatcaacaataaaacaaatttctacacagttcctggcatgctggaacctttactctgtgggaacagttctgatgctgggcaatgcccagagggataccagtgtatgaaagcaggaaggaaccccaactatggttacacaagttttgacacttttagctgggccttcttggcattatttcgccttatgacccaggactattgggaaaacttgtatcaattgactttacgagcagccgggaaaacatacatgatcttcttcgtcttggtcatctttgtgggttctttctatctggtgaacttgatcttggctgtggtggccatggcttatgaagaacagaatcaggcaacactggaggaggcagaacaaaaagaggctgaatttaaagcaatgttggagcaacttaagaagcaacaggaagaggcacaggctgctgcgatggccacttcagcaggaactgtctcagaagatgccatagaggaagaaggtgaagaaggagggggctcccctcggagctcttctgaaatctctaaactcagctcaaagagtgcaaaggaaagacgtaacaggagaaagaagaggaagcaaaaggaactctctgaaggagaggagaaaggggatcccgagaaggtgtttaagtcagagtcagaagatggcatgagaaggaaggcctttcggctgccagacaacagaatagggaggaaattttccatcatgaatcagtcactgctcagcatcccaggctcgcccttcctctcccgccacaacagcaagagcagcatcttcagtttcaggggacctgggcggttccgagacccgggctccgagaatgagttcgcggatgacgagcacagcacggtggaggagagcgagggccgccgggactccctcttcatccccatccgggcccgcgagcgccggagcagctacagcggctacagcggctacagccagggcagccgctcctcgcgcatcttccccagcctgcggcgcagcgtgaagcgcaacagcacggtggactgcaacggcgtggtgtccctcatcggcggccccggctcccacatcggcgggcgtctcctgccagaggctacaactgaggtggaaattaagaagaaaggccctggatctcttttagtttccatggaccaattagcctcctacgggcggaaggacagaatcaacagtataatgagtgttgttacaaatacactagtagaagaactggaagagtctcagagaaagtgcccgccatgctggtataaatttgccaacactttcctcatctgggagtgccacccctactggataaaactgaaagagattgtgaacttgatagttatggacccttttgtggatttagccatcaccatctgcatcgtcctgaatacactgtttatggcaatggagcaccatcctatgacaccacaatttgaacatgtcttggctgtaggaaatctggttttcactggaattttcacagcggaaatgttcctgaagctcatagccatggatccctactattatttccaagaaggttggaacatttttgacggatttattgtctccctcagtttaatggaactgagtctagcagacgtggaggggctttcagtgctgcgatctttccgattgctccgagtcttcaaattggccaaatcctggcccaccctgaacatgctaatcaagattattggaaattcagtgggtgccctgggcaacctgacactggtgctggccattattgtcttcatctttgccgtggtggggatgcaactctttggaaaaagctacaaagagtgtgtctgcaagatcaaccaggactgtgaactccctcgctggcatatgcatgactttttccattccttcctcattgtctttcgagtgttgtgcggggagtggattgagaccatgtgggactgcatggaagtggcaggccaggccatgtgcctcattgtctttatgatggtcatggtgattggcaacttggtggtgctgaacctgtttctggccttgctcctgagctccttcagtgcagacaacctggctgccacagatgacgatggggaaatgaacaacctccagatctcagtgatccgtatcaagaagggtgtggcctggaccaaactaaaggtgcacgccttcatgcaggcccactttaagcagcgtgaggctgatgaggtgaagcctctggatgagttgtatgaaaagaaggccaactgtatcgccaatcacaccggtgcagacatccaccggaatggtgacttccagaagaatggcaatggcacaaccagcggcattggcagcagcgtggagaagtacatcattgatgaggaccacatgtccttcatcaacaaccccaacttgactgtacgggtacccattgctgtgggcgagtctgactttgagaacctcaacacagaggatgttagcagcgagtcggatcctgaaggcagcaaagataaactagatgacaccagctcctctgaaggaagcaccattgatatcaaaccagaagtagaagaggtccctgtggaacagcctgaggaatacttggatccagatgcctgcttcacagaaggttgtgtccagcggttcaagtgctgccaggtcaacatcgaggaagggctaggcaagtcttggtggatcctgcggaaaacctgcttcctcatcgtggagcacaactggtttgagaccttcatcatcttcatgattctgctgagcagtggcgccctggccttcgaggacatctacattgagcagagaaagaccatccgcaccatcctggaatatgctgacaaagtcttcacctatatcttcatcctggagatgttgctcaagtggacagcctatggcttcgtcaagttcttcaccaatgcctggtgttggctggacttcctcattgtggctgtggtggtgaatgccttggtgggcgccatcccctccatcatgaatgtgctgctggtgtgtctcatcttctggctgattttcagcatcatgggagttaacttgtttgcgggaaagtaccactactgctttaatgagacttctgaaatccgatttgaaattgaagatgtcaacaataaaactgaatgtgaaaagcttatggaggggaacaatacagagatcagatggaagaacgtgaagatcaactttgacaatgttggggcaggatacctggcccttcttcaagtagcaaccttcaaaggctggatggacatcatgtatgcagctgtagattcccggaagcctgatgagcagcctaagtatgaggacaatatctacatgtacatctattttgtcatcttcatcatcttcggctccttcttcaccctgaacctgttcattggtgtcatcattgataacttcaatcaacaaaagaaaaagttcggaggtcaggacatcttcatgaccgaagaacagaagaagtactacaatgccatgaaaaagctgggctcaaagaagccacagaaacctattccccgccccttgaacaaaatccaaggaatcgtctttgattttgtcactcagcaagcctttgacattgttatcatgatgctcatctgccttaacatggtgacaatgatggtggagacagacactcaaagcaagcagatggagaacatcctctactggattaacctggtgtttgttatcttcttcacctgtgagtgtgtgctcaaaatgtttgcgttgaggcactactacttcaccattggctggaacatcttcgacttcgtggtagtcatcctctccattgtgggaatgttcctggcagatataattgagaaatactttgtttccccaaccctattccgagtcatccgattggcccgtattgggcgcatcttgcgtctgatcaaaggcgccaaagggattcgtaccctgctctttgccttaatgatgtccttgcctgccctgttcaacatcggccttctgctcttcctggtcatgttcatcttctccatttttgggatgtccaattttgcatatgtgaagcacgaggctggtatcgatgacatgttcaactttgagacatttggcaacagcatgatctgcctgtttcaaatcacaacctcagctggttgggatggcctgctgctgcccatcctaaaccgcccccctgactgcagcctagataaggaacacccagggagtggctttaagggagattgtgggaacccctcagtgggcatcttcttctttgtaagctacatcatcatctctttcctaattgtcgtgaacatgtacattgccatcatcctggagaacttcagtgtagccacagaggaaagtgcagaccctctgagtgaggatgactttgagaccttctatgagatctgggagaagttcgaccccgatgccacccagttcattgagtactgtaagctggcagactttgcagatgccttggagcatcctctccgagtgcccaagcccaataccattgagctcatcgctatggatctgccaatggtgagcggggatcgcatccactgcttggacatcctttttgccttcaccaagcgggtcctgggagatagcggggagttggacatcctgcggcagcagatggaagagcggttcgtggcatccaatccttccaaagtgtcttacgagccaatcacaaccacactgcgtcgcaagcaggaggaggtatctgcagtggtcctgcagcgtgcctaccggggacatttggcaaggcggggcttcatctgcaaaaagacaacttctaataagctggagaatggaggcacacaccgggagaaaaaagagagcaccccatctacagcctccctcccgtcctatgacagtgtaactaaacctgaaaaggagaaacagcagcgggcagaggaaggaagaagggaaagagccaaaagacaaaaagaggtcagagaatccaagtgttagaggagaacaaaaattcagtattatacagatctaaaactcgcaagtgaaagattgtttacaaacttcctgaatattatcaatgcagaacagctgtggagactctaacctgaagatctataccaaacgtcgtctgcttaccacgtaacacagctgcatcttgagcagtgacctgccaagggcaaaggaccccgctccctagacttacagattttctaatgcttgggcaggtggttactgcatgttccacatcagtcaatgcaacttaggacaaaactaaccagatacagaaacagaagagaggctgccgggaccagcatatttccgttgcagccaaatggattttattttttcattttattgattctcagaagcagaaagcatcactttaaaagttcgtttgttcatgcaaactatatttgcattcttacattagttaagctaagcagcaaaaagaaaacacacacacacactcacatttagcccatgtcatttaattgtcagtttctttgacataaagcgcatcttctccacatgggcttcacgtggtttggagatgggtgggggaaaacaatcaggtttcttcaggctgaggaggacttgctcaggccgattccaaacattgtgctcgttcaatgcgtagaaatgatttgcatgatggcatgccgtgatcagaagtcatgcatgagatccatacaccacaggacactactaatctagtcccttgcactgggtcagcctttggacaggacccagccctgcaccgttcactgtatttggagaaaatggtaagagttccataccggctacaattctttgagttcttaaaagtccttcatacaccttctgggtagggaaacaaccaactaattgactaacaccaccaacaacaaaaaacaaacccaatccaacaagcagatggatccgttgcgtgtatatgtttaacagacatctctaacatacagccattgttgcacattttgcaagatgaactatttaatgctgctctgtgtccagtacatgggggagactttgatcccaaatggcttgtactatttatgtcactgtaaaaccaaatcctagggctaaaaaaaaaattcatttgtatcttgcaatatttatgacgatcgtttagccttcatactggagaattgacacttatttggggtagagataaaagtgcttttcaaagtagcaccagttatctctaggggtaatttggggaacttaaaatgacctttcattgggagttatggggtgctcatttcatttcgtatcatgtcctctgcattgtggctttctccaggcatgggtcgataccgcgaggggttcagatattctgaactgacctctctcttctgcagaggggtcatcgcatactcacacactgcatggctccccctgcctccatcacatttctccactgagcagggcttcccttgcccacagaggtgggtgagggtagcagcattggggttacggctgtggtttctttcacttattattagaataatagtcactggtgagacctgaggtagagatggagccgcctctgaaccaagcccacttggtttctttactgcactggttttcatgagaaagcaatttaatattaattctttaggatgttccagcttccctcagtgcattcccttgcagccagttttgctatctgcttagcgacctgcccattgtcattgaagtgtggcttctgggggagcgtcatatcttccattccatctcctgctttaggaaagacagtttttagtccaggggtttctcacctaatgactggatttcaaagccaagatgctgcagttgaatgtgtcaggaagtctctatacaaagagtgaaaagagactttagcatgtgaaccaaatccaaataaattcttcctgaaccatacactagagggggaagaagaaccaacccataatcagacacaccgatctgccttgcagtagaagcactttgaaggtaatttcacagtccaccagactagttttgcatagaacaaaccacagtaagtcggcaagcagagctttctatcttgttggaccactagaaactgtccagctctcttaagcctgcttctgcagcagcatccgtagaatttttgtactacacccaataccagactgggaaggcctatctttcagtgaccctgctaactgctaggatgaatgtatagtaacatgtacaggctacattgtaaacagcacaaaacagaagctgacatgtgtggttattctttttaagagaattataaatactgtaatatataattttattttattggcatgcctttttgtaaatacaaattattaacttattaaataggaaatggcttctggcttatgccattgtcatgtttatttttattttttatacttttctttcttcttagaacttagatgctgtcagccaatgtacatccccaaaccagacagacagacaaaaaattttttattgctaatggtcttttccaaaacaacaaaaaatatatatttttgttccaatgtgcaataaattctaaccacgtctatgcaagatttggctaaacttaataattgttcttaggtcttgagatgggcaacagagctgtaagatcccgccaagtacaccatgtgctcatgctagtgatgtcattaagctactagggcatagtgatgagatttttctgagatctaaccgtttccctccgctagctcctgaccccagcccagcaggctgtctcctcagtaacacatgggccttggtatctggcacccatcagccagcctcataggaaagaagccacctccgctgtatttgaagtttcactttttaaaaaaaaaatgtttcccacctttttatccttcacccactgtccttccacctgctcactcactcactctctcacccatcctgctccacacttctttggtagtaaatgacaccatcaccagcagtttacacccgcagttaacaggcatcgaactgaaagaatcagtgatgacgtttttgtgcatcttcgctgagtgggtaagtggcaatgctttgccaaatattaacgaagccaatcaaagagcagcgcagccacagtatcactgcacatatatatatagatatataaatataatataaatatttattttttgtagccaggtccttggtgcagtatttatactccacaatccaccttttaaaaaaaggacaaaaagcaaaaagatgtgtgatgctgttaataatttaaaatgcagagccaaagccactgagcagcagctgacatggttgctggtttgtgtgtgaaaaacattttccccctcttctttccttaacctccttgttgcttaggatgaggagactctgtaatttaaagcagaggacgagaggaggaagaaacttccatgttaacatggcctactctcactcctttctaaacgagccacttttggcgtcagaagttgactggagagagataaacaaactcccagtcaaagcccctaaagcgacagtgcccagagttcttttattttttgctgcaaccaacgtaaacctgtaaaagaccaacagtgaagattagtgtattagtgaatgcatggaggccaatgctgccctaatgagacgtgataaaaagtccatcactcctctaagccaaaaggaaacaaaataaaaatgaccctttttactgtacaagggaagcctgtcttggtgtgtttgttgcttgactatcccaattcttgagtcctgggaggggtctgggaattgggaccttagcacatttggggaaaggaggggaatagagggaagagaaatcaaaggggctctcgcaaagttctggctaagaaggagagattggaagaaaagcaaggtcctgaccccccccagatccccactcacccattgcatgtggcattccccactactggccggtgcttagaggcccataaaacctctcttcacctaatccacctggcagagccctcagagatgacgtcagccaacccagctggactggccttacttctgcctttttcacctgtatgttaaaaaacaaaaacaaaaaaaccctatccctgtgccccaaagctagggcatgtgcgatgcccaacagcctccactctgcaagctcatactgggccctgccctccatcgccccagacaggcccacagacgagggctggtctccttcacatttgtgtgggggtgggggcacgcttcctcttccttctctccctgtccccctttggggacatccctctctaggcctaggccatgtctcctggttcaggaacactctctcaggaggatctcgaagtgcccactctagtccgctccagtagctccactctggagcgagatggctggtaggtcagtccccatcctcctcccctcggcccccagcctccaggcctgctgtggaggtgagatcgccatgggtcatgactttgtgacttgagtcctgggcatcttctgtaatgatgcctctttcttttctcagatgttgcccacagtttagcaaaaagcttcgttcattttcaggtactccccaccccctaaagaattggctgcaacttgccagccagggggacatcaggagagaagaggaagtctgggttgggaggggcccaagttttgcaacagagtttccattgttcagctctgtgctttcttggattccctttccaagattaccaagatttcctccattaaaactcctgaggcaccccctgcccctgccccccactgctgtttgctgtacatagaggctaagtggggtggggtgggtgggtgtgcacatgtggtgtgtgtgtgtgtgtgtgaagaatgtatataggtaaaggggagtgtggtccagtggttccagaaaagggacagaactgtgttctgtccctagcttgaccgctggctcgctgtgtggccttgggcaagtcacttaacctctctgtgcctcagtttccccatctgtaaaatggggataataatactgacctacctcacaggggtgttgtgaggctttaactaaatgttttgtaaagcgttttgagatacagaggcaaaggcgctagggaagggcaaagtattattggacttaaggaagctgaaatggtaccataaatgctgctattctgagagccattttaaactgcactgctccaaccacccccgcttctcatcaccaggacagcaggtcgagaaaatgtctttcctttcacttgcttctggggtttgtgattattcaagacaattttttccccttgctgtatctccttccctcacctcctcgacttgttccctgttctgtttatgcggaaatggcagaaatgcttgagaaatgagaatgtgtaagtggattggaagtttatagtatgaaatttcaattttactttgtactgtacatctttttactttagaatttgcaataaagggttacgtcaatcttgttttcaactctcaaaaaaaaaaaaaa

SEQ ID NO: 23 - DNA, 3459 bp, Homo sapiens
  misc_feature (1)..(3459): Homo sapiens hyperpolarization activated cyclic nucleotide-gated potassium channel 2 (HCN2)
ggccggcggcggcggcggcggctccgctccgcactgcccggcgccgcctcgccatggacgcgcgcgggggcggcgggcggcccggggagagcccgggcgcgacccccgcgccggggccgccgccgccgccgccgcccgcgcccccccaacagcagccgccgccgccgccgccgcccgcgccccccccgggccccgggcccgcgcccccccagcacccgccccgggccgaggcgttgcccccggaggcggcggatgagggcggcccgcggggccggctccgcagccgcgacagctcgtgcggccgccccggcaccccgggcgcggcgagcacggccaagggcagcccgaacggcgagtgcgggcgcggcgagccgcagtgcagccccgcggggcccgagggcccggcgcgggggcccaaggtgtcgttctcgtgccgcggggcggcctcggggcccgcgccggggccggggccggcggaggaggcgggcagcgaggaggcgggcccggcgggggagccgcgcggcagccaggccagcttcatgcagcgccagttcggcgcgctcctgcagccgggcgtcaacaagttctcgctgcggatgttcggcagccagaaggccgtggagcgcgagcaggagcgcgtcaagtcggcgggggcctggatcatccacccgtacagcgacttcaggttctactgggacttcaccatgctgctgttcatggtgggaaacctcatcatcatcccagtgggcatcaccttcttcaaggatgagaccactgccccgtggatcgtgttcaacgtggtctcggacaccttcttcctcatggacctggtgttgaacttccgcaccggcattgtgatcgaggacaacacggagatcatcctggaccccgagaagatcaagaagaagtatctgcgcacgtggttcgtggtggacttcgtgtcctccatccccgtggactacatcttccttatcgtggagaagggcattgactccgaggtctacaagacggcacgcgccctgcgcatcgtgcgcttcaccaagatcctcagcctcctgcggctgctgcgcctctcacgcctgatccgctacatccatcagtgggaggagatcttccacatgacctatgacctggccagcgcggtgatgaggatctgcaatctcatcagcatgatgctgctgctctgccactgggacggctgcctgcagttcctggtgcctatgctgcaggacttcccgcgcaactgctgggtgtccatcaatggcatggtgaaccactcgtggagtgaactgtactccttcgcactcttcaaggccatgagccacatgctgtgcatcgggtacggccggcaggcgcccgagagcatgacggacatctggctgaccatgctcagcatgattgtgggtgccacctgctacgccatgttcatcggccacgccactgccctcatccagtcgctggactcctcgcggcgccagtaccaggagaagtacaagcaggtggagcagtacatgtccttccacaagctgccagctgacttccgccagaagatccacgactactatgagcaccgttaccagggcaagatgtttgacgaggacagcatcctgggcgagctcaacgggcccctgcgggaggagatcgtcaacttcaactgccggaagctggtggcctccatgccgctgttcgccaacgccgaccccaacttcgtcacggccatgctgaccaagctcaagttcgaggtcttccagccgggtgactacatcatccgcgaaggcaccatcgggaagaagatgtacttcatccagcacggcgtggtcagcgtgctcactaagggcaacaaggagatgaagctgtccgatggctcctacttcggggagatctgcctgctcacccggggccgccgcacggcgagcgtgcgggctgacacctactgccgcctctattcgctgagcgtggacaacttcaacgaggtgctggaggagtaccccatgatgcggcgcgccttcgagacggtggccatcgaccgcctggaccgcatcggcaagaagaattccatcctcctgcacaaggtgcagcatgacctcaactcgggcgtattcaacaaccaggagaacgccatcatccaggagatcgtcaagtacgaccgcgagatggtgcagcaggccgagctgggtcagcgcgtgggcctcttcccgccgccgccgccgccgccgcaggtcacctcggccatcgccacgctgcagcaggcggcggccatgagcttctgcccgcaggtggcgcggccgctcgtggggccgctggcgctcggctcgccgcgcctcgtgcgccgcccgcccccggggcccgcacctgccgccgcctcacccgggcccccgccccccgccagccccccgggcgcgcccgccagcccccgggcaccgcggacctcgccctacggcggcctgcccgccgccccccttgctgggcccgccctgcccgcgcgccgcctgagccgcgcgtcgcgcccactgtccgcctcgcagccctcgctgcctcacggcgcccccggccccgcggcctccacacgcccggccagcagctccacaccgcgcttggggcccacgcccgctgcccgggccgccgcgcccagcccggaccgcagggactcggcctcacccggcgccgccggcggcctggacccccaggactccgcgcgctcgcgcctctcgtccaacttgtgaccctcgccgaccgccccgcgggcccaggcgggccgggggcggggccgtcatccagaccaaagccatgccattgcgctgccccggccgccagtccgcccagaagccatagacgagacgtaggtagccgtagttggacggacgggcagggccggcggggcagccccctccgcgcccccggccgtcccccctcatcgccccgcgcccacccccatcgcccctgcccccggcggcggcctcgcgtgcgagggggctcccttcacctcggtgcctcagttcccccagctgtaagacagggacggggcggcccagtggctgagaggagccggctgtggagccccgcccgccccccaccctctaggtggcccccgtccgaggaggatcgttttctaagtgcaatacttggcccgccggcttcccgctgcccccatcgcgctcacgcaataaccggcccggcccccgtccgcgcgcgtcccccggtgacctcggggagcagcaccccgcctccctccagcactggcaccgagaggcaggcctggctgcgcagggcgcgggggggaggctggggtcccgccgccgtgatgaatgtactgacgagccgaggcagcagtgcccccaccgtggccccccacgccccattaacccccacacccccattccgcgcaataaacgacagcattggcgccaaaaaaaaaaaaaaaaaaaaaaaaaaaaaaaaaaaaaaaaaaaaaaaaaaaaa

SEQ ID NO: 24 - DNA, 3195 bp, Mus musculus
  misc_feature (1)..(3195): Mus musculus hyperpolarization-activated, cyclic nucleotide-gated K+ 2 (Hcn2)
ccgctccgctccgcactgcccggcgccgcctcgccatggatgcgcgcgggggcggcgggcggccgggcgatagtccgggcacgacccctgcgccggggccgccgccaccgccgccgccgcccgcgccccctcagcctcagccaccacccgcgccacccccgaaccccacgaccccctcgcacccggagtcggcggacgagcccggcccgcgcgcccggctctgcagccgcgacagcgcctgcacccctggcgcggccaagggcggcgcgaatggcgagtgcgggcgcggggagccgcagtgcagccccgagggccccgcgcgcggccccaaggtttcgttctcatgccgcggggcggcctccgggccctcggcggccgaggaggcgggcagcgaggaggcgggcccggcgggtgagccgcgcggcagccaggctagcttcctgcagcgccaattcggggcgcttctgcagcccggcgtcaacaagttctccctgcggatgttcggcagccagaaggccgtggagcgcgagcaggaacgcgtgaagtcggcgggggcctggatcatccacccctacagcgacttcaggttctactgggacttcaccatgctgttgttcatggtgggaaatctcattatcattcccgtgggcatcactttcttcaaggacgagaccaccgcgccctggatcgtcttcaacgtggtctcggacactttcttcctcatggacttggtgttgaacttccgcaccggcattgttattgaggacaacacggagatcatcctggaccccgagaagataaagaagaagtacttgcgtacgtggttcgtggtggacttcgtgtcatccatcccggtggactacatcttcctcatagtggagaagggaatcgactccgaggtctacaagacagcgcgtgctctgcgcatcgtgcgcttcaccaagatcctcagtctgctgcggctgctgcggctatcacggctcatccgatatatccaccagtgggaagagattttccacatgacctacgacctggcaagtgcagtgatgcgcatctgtaacctgatcagcatgatgctactgctctgccactgggacggttgcctgcagttcctggtgcccatgctgcaagacttccccagcgactgctgggtgtccatcaacaacatggtgaaccactcgtggagcgagctctactcgttcgcgctcttcaaggccatgagccacatgctgtgcatcggctacgggcggcaggcgcccgagagcatgacagacatctggctgaccatgctcagcatgatcgtaggcgccacctgctatgccatgttcattgggcacgccactgcgctcatccagtccctggattcgtcacggcgccaataccaggagaagtacaagcaagtagagcaatacatgtccttccacaaactgcccgctgacttccgccagaagatccacgattactatgaacaccggtaccaagggaagatgtttgatgaggacagcatccttggggaactcaacgggccactgcgtgaggagattgtgaacttcaactgccggaagctggtggcttccatgccgctgtttgccaatgcagaccccaacttcgtcacagccatgctgacaaagctcaaatttgaggtcttccagcctggagattacatcatccgagaggggaccatcgggaagaagatgtacttcatccagcatggggtggtgagcgtgctcaccaagggcaacaaggagatgaagctgtcggatggctcctatttcggggagatctgcttgctcacgaggggccggcgtacggccagcgtgcgagctgacacctactgtcgcctctactcactgagtgtggacaatttcaacgaggtgctggaggaataccccatgatgcggcgtgcctttgagactgtggctattgaccggctagatcgcataggcaagaagaactccatcttgctgcacaaggttcagcatgatctcagctcaggtgtgttcaacaaccaggagaatgccatcatccaggagattgtcaaatatgaccgtgagatggtgcagcaggcagagcttggccagcgtgtggggctcttcccaccaccgccaccaccgcaggtcacatcggccattgccaccctacagcaggctgtggccatgagcttctgcccgcaggtggcccgcccgctcgtggggcccctggcgctaggctccccacgcctagtgcgccgcgcgcccccagggcctctgcctcctgcagcctcgccagggccacccgcagcaagccccccggctgcaccctcgagccctcgggcaccgcggacctcaccctacggtgtgcctggctctccggcaacgcgtgtggggcccgcattgcccgcacgtcgcctgagccgcgcctcgcgcccactgtccgcctcgcagccctcgctgccccatggcgtgcccgcgcccagccccgcggcctctgcgcgcccggccagcagctccacgccgcgcctgggacccgcacccaccgcccggaccgccgcgcccagtccggaccgcagggactcagcctcgccgggcgctgccagtggcctcgacccactggactctgcgcgctcgcgcctctcttccaacttgtgacccttgagcgccgccccgcgggccgggcggggccgtcatccacaccaaagccatgcctcgcgccgcccgcccgtgcccgtgcagaagccatagagggacgtaggtagcttaggaggcgggcggccctgcgcccggctgtccccccatcgccctgcgcccacccccatcgcccctgccccagcggcggccgcacgggagagggaggggtgcgatcacctcggtgcctcagccccaacctgggacagggacagggcggccctggccgaggacctggctgtgccccgcatgtgcggtggcctccgaggaagaatatggatcaagtgcaatactcggcccgccggcttcccgctgcccctggcaagctcacgcaataaccagcccgcccctgcccacgcgtccgtggtgacctccctcgggcacccaggggcgggcttcacggccaagccggcgtgggggtgaggctgggtccccgccgtcgccatgaatgtactgacgagccgaggcagcagtggcccccacgccccattaacccacaaccccattccgcgcaataaacgacagcattggc

SEQ ID NO: 25 - DNA, 9685 bp, Homo sapiens
  misc_feature (1)..(9685): Homo sapiens hyperpolarization activated cyclic nucleotide-gated potassium channel 1 (HCN1)
ccgtcgccggccgcgtcctccgggcatggaaggaggcggcaagcccaactcttcgtctaacagccgggacgatggcaacagcgtcttccccgccaaggcgtccgcgacgggcgcggggccggccgcggccgagaagcgcctgggcaccccgccggggggcggcggggccggcgcgaaggagcacggcaactccgtgtgcttcaaggtggacggcggtggcggcggtggcggcggcggcggcggcggcgaggagccggcggggggcttcgaagacgccgaggggccccggcggcagtacggcttcatgcagaggcagttcacctccatgctgcagcccggggtcaacaaattctccctccgcatgtttgggagccagaaggcggtggaaaaggagcaggaaagggttaaaactgcaggcttctggattatccacccttacagtgatttcaggttttactgggatttaataatgcttataatgatggttggaaatctagtcatcataccagttggaatcacattctttacagagcaaacaacaacaccatggattattttcaatgtggcatcagatacagttttcctattggacctgatcatgaattttaggactgggactgtcaatgaagacagttctgaaatcatcctggaccccaaagtgatcaagatgaattatttaaaaagctggtttgtggttgacttcatctcatccatcccagtggattatatctttcttattgtagaaaaaggaatggattctgaagtttacaagacagccagggcacttcgcattgtgaggtttacaaaaattctcagtctcttgcgtttattacgactttcaaggttaattagatacatacatcaatgggaagagatattccacatgacatatgatctcgccagtgcagtggtgagaatttttaatctcatcggcatgatgctgctcctgtgccactgggatggttgtcttcagttcttagtaccactactgcaggacttcccaccagattgctgggtgtctttaaatgaaatggttaatgattcttggggaaagcagtattcatacgcactcttcaaagctatgagtcacatgctgtgcattgggtatggagcccaagccccagtcagcatgtctgacctctggattaccatgctgagcatgatcgtcggggccacctgctatgccatgtttgtcggccatgccaccgctttaatccagtctctggattcttcgaggcggcagtatcaagagaagtataagcaagtggaacaatacatgtcattccataagttaccagctgatatgcgtcagaagatacatgattactatgaacacagataccaaggcaaaatctttgatgaggaaaatattctcaatgaactcaatgatcctctgagagaggagatagtcaacttcaactgtcggaaactggtggctacaatgcctttatttgctaatgcggatcctaattttgtgactgccatgctgagcaagttgagatttgaggtgtttcaacctggagattatatcatacgagaaggagccgtgggtaaaaaaatgtatttcattcaacacggtgttgctggtgtcattacaaaatccagtaaagaaatgaagctgacagatggctcttactttggagagatttgcctgctgaccaaaggacgtcgtactgccagtgttcgagctgatacatattgtcgtctttactcactttccgtggacaatttcaacgaggtcctggaggaatatccaatgatgaggagagcctttgagacagttgccattgaccgactagatcgaataggaaagaaaaattcaattcttctgcaaaagttccagaaggatctgaacactggtgttttcaacaatcaggagaacgaaatcctcaagcagattgtgaaacatgacagggagatggtgcaggcaatcgctcccatcaattatcctcaaatgacaaccctgaattccacatcgtctactacgaccccgacctcccgcatgaggacacaatctccaccggtgtacacagcgaccagcctgtctcacagcaacctgcactcccccagtcccagcacacagaccccccagccatcagccatcctgtcaccctgctcctacaccaccgcggtctgcagccctcctgtacagagccctctggccgctcgaactttccactatgcctcccccaccgcctcccagctgtcactcatgcaacagcagccgcagcagcaggtacagcagtcccagccgccgcagactcagccacagcagccgtccccgcagccacagacacctggcagctccacgccgaaaaatgaagtgcacaagagcacgcaggcgcttcacaacaccaacctgacccgggaagtcaggccactctccgcctcgcagccctcgctgccccatgaggtgtccactctgatttccagacctcatcccactgtgggcgagtccctggcctccatccctcaacccgtgacggcggtccccggaacgggccttcaggcagggggcaggagcactgtcccgcagcgcgtcaccctcttccgacagatgtcgtcgggagccatccccccgaaccgaggagtccctccagcaccccctccaccagcagctgctcttccaagagaatcttcctcagtcttaaacacagacccagacgcagaaaagccacgatttgcttcaaatttatgatccctgctgattgtcaaagcagaaagaaatactctcataaactgagactatactcagatcttattttattctatctcctgatagatccctctagcctactatgaagagatattttagacagctgtggcctacacgtgaaatgtaaaaatatatatacatatactataaaatatatatctaaattcccaagagagggtcaaaagacctgtttagcattcagtgttatatgtcttcctttctttaaatcattaaaggatttaaaatgtcgttgtaagattatttatttctaacctacttttacttaagtcctttgatatgtatatttctctattttatgaagagttcttggattcaatggaaacaaaactgattttaaaaaggcaactcaaatgaactagtaaatagcaccaatcaaaactttctttcattagctgtgtctctgcatctaaattgttaatcattaatggtggagaattaaataacaaatcccattttatagatctaaattgtatttcggtgctttcaatttcaaattaggttaaagaatgcactacttgcttggccaccgtaggagactagcattgccactgtttgttaagaatatcactaacctcaaacatgttcattgatctttcagaaagctgagggaaaattaatatttgtcttcatgtgttatcggacttttaccaagactcgatcaatgttagttgtaaataactttttcaacccaaataaaaatagctattctgtgttgtatgaaggtaaaagtcattgtttaagaatttagttttattgcttcacttcaaaacttagagttttaaattttacaaaacctgattgtgagaattattcaattgtaatgcaatggcttgaaacctacaatattattttaacctgcaatgttttatgcaaaattgtatgctcgaatctacaaattgcttgtattacaccaaaaatcgttacttttctttcttcttgccataatcaagcatctgaaaatagtccctgcatgcttctggggaaaaaaaaataggttcaaatcagtcattgtagagaaaggcttacagtatttctcgtttctagaaaagcataacaattcattaattgcctatcttataattcctataaggctgacttggatctctgacttaagtctagaagtgaggttttcacttttatttaatattattgctattattatttaaataattatttgtaaaccacagcttgatttggagttaagtgttgtctgtgtcttcactgtagctggtaatttactatggtattaatttaaagaaataaattcaatacagcatcacgtctgtttgggaacacacattttgctctatgtatattgtaactaaatggttagtaaactttaaggcatgtggtgactaacgtagcccttagataggaggtttgtcatttttaatgtgttgaaaatagtacagacatggtgacagaaagcaggggtaaagttgaacgtctatgagcattgtctgtctgttttcttaactctttacatgatctgtgagcacctgcttcttctcaaagaactcccttagtaatcatttgtgcctaatgcatcccacacatgtggagtatgactggaacacaattgaaagattcctaacaactggctgcggtaaataacccaagttaagagcatgacttaaagtggaggtcttcagtatctctggcactggcaatacatttaagaacatacagagatcacacattatgtgggacagggcctgcagatagacttgaggacagaaaacaaaaacaaaagcagaaactctaaagtggatccttgctgcctgttatgccttttgtggatataatacctataataccaaaatgagttgttatgtttaatttcaaacaaatcccaactggtgtggaagtaattaactgctagtcgatgtaatgtaatcatcaatatgctggtgacatatgatggcatgtcaggaatactgttgtaagaaagggattgttcagtctacctagaacctatgggaaagctgcaagaccatttacatatcagtacaaaactcctttattttattaaaatatgagaattaacttcaatttattaagattacgtattggtcctttaaattattaaaggtttacatttgataggattaatggatttggtagtgaaatatttttatatatataaatttttggttttttattttgagcactattgggaaacataagcaaaattgaaatgcatggtctttcagtgcaaccatacaatttttattcactttgtaatagaatggacaaaaaaactaaggtttatatattaaactaataaaaaggtacagggtaaattatatacatatatgtatgaatgtatatatacttagattaagaaaaaaaaatgactcacattcctgtaatataaaggtccattgctagttgaaaagtgacctcttttctctgtacatagtgaagcccgtatgtatggagttttaatctgtacagaaagaaatgtattattcattggtctttctgctgctggaaagataatagaatagctgattcttacaacaactgtatgatcagggatgagttagagtatttcttttgtctttgctcaaagccatacatatataaatatatataaagattattaataaattcaacaaaaaataaactacaatcatgaatattttattttatcaatatatgctaatcaaatgttttattttattaatgagtacagctttgcctctatttttacatcataaaatttccaagagcttatacttgataatcacataaaatcaattatattctataattctggaatgagaggactttaaatagaacatggatttttcaaccacttctttgtttacctttttcgtatagtaaaatatgagcttattttcttttttgctctgtgcctttttagaatgtggttgttctccaacttcaaaacacaaccattttgtgtaattattggatatacagatgagaaataattttcctgtaataatggtaaataattgtcctgtaatatatttatacaaacctattttgttataatagttctatgacatacctattttgttataatagttttataacatacctattttgttataatagggccttgggctctattataacaaaacaggtttatataaatacattcatatttatagccttaaacaaaataggtttgtatagtaggcccaaggccctattataacaaaataggtttgtataaatatattcatatttatagccttgggccctgcaaagttatttggagagtgatagaacctccccacacaagtgggttttaaatgatacctaaaagcctgagatagagtttatcataatgagaataagtagtgaagcattcctgaaaacaaggaggataaatagtagcaatggcaatatattctattcaccaaaaaagtagcttgtctccaggtctggatgacctcttcataagtgaacaattccctgatgaatgctgttccactttaaagagatggttattttgcaatttataaacttaactgagcatactattttcaggacactacaaagtacatagtcaagttattaccgtcattcctataggacagagaatgatttaaacaatatgacatgtttaaaattttagatgatttatttcaaatcctgaattatgtctacttcaaattatgagcaatctctattatctttcaggactatttaaaaaatctccatacctcctctttcagcttaggtttttatatttgttaattttactatgttactttgcttctatggccctcacttgcactgtttataatgttaacctgtttgaatgagatatctctccattatatctttatctttttagaacatgtgccataaaacccatgtggttattttgttcatatgcactacaactagcaaacttttattttatgttcacctactttaaaaagtactcatactgtaccatgggaaagaaacatgggccttatagaatggaatgctgagctattttgagtcctgaaaacaatggccaaaaatttaaggaactgtggttgctgcatgcaccaaattgtagcacatacaaataaataaaatacagttctataactgtattcaatatggttattttcaaggaaaaataggatagaaattttctttgaaagatttgctattgtacttagggaagtacacctgctattacccctcaaaagcaatgactttcttaagcaatttcaatttttttcctttttaacaaccctgcacttgcacaaccctgcatctcttgtctcaccctaatgctgaccctgcttccaattcaggctttgacagggacaaatggagattttaaggttttttaatgagagaagccttgctgacactccccgcatcccaccttgaaaattatttaaagatgatcttaaaagaacctacagataatgctctgaatttgtttacaaggtccttgatgtggagagaatgagtaatacattaaatgctggggaaataattatcatttagtgaaaaattaagcatgcttttctaaaatgtggatgtttaacagagcacagctgattgggaacactggcagttcaatgcagctggagtgtctcacagggtgaagaggaacccagatcaagtgggaaatagggatttcaaaaaccttctgaaaggcttttaacatcagggcaaaggttttacagtgttgtactttatattcttaatttaagcttatcaacggggaggattcacattgaaatgtctgttttgctaatgtaacaaggaataaaataatttatgacacagcaaaaactgggggctatttacaaccccaccctgccttgtgaggatgctgggtcctaaggaaggtcctaggtttgctaaatagagtaatgggaataggaaactgaaagtttacttgctttgcgtaagataatatattgcggcagggaaggggtacaagaagataagagctagaacctgtgagaagggaaagaagtgataaaataattttggctaaaataaagcaagttctcaggccaggcgtggtggctcatgcctgtaatcctagcactttgggaggccaaagtgggaggaatgcttgaggccaggaattggagaccaaccagggcagcatgtcaagacctggtctctacaaggaaaaaaaatttttttaattgaccagtatatacttacatacctgcagtgtttagaaaacccgactataggtggtgtattcctgtagtcctagttactcagaaagttaaggcaggaggatcgcttgaacttaggaggtcaaggctgcagtgagccctgttcgttccactgcactccagcttgggggacagagcaaaaccctttctcaagaaaaacccaaacaaacaaaaaagtgaagccagttctttaatttctattggagatatcacacacgctccaaagagtggcaaaggtgaacattctttggagctattatatttctaatgtaacatcaatggccatttactcccaaagggaaacatctttttttttttttttttttaagatggagtctggctctgtcacccaggtgggagtgcaatggcaggatcttggcccactgcaacatcagcctcccaggtttaagcaattctcctgcatcagcctccagagtagctgggattacaggtgcccgccaccatggctagctaaatttttgtaatttttagtagagacagggttttaccatgttggccaggccggtctcgaacccctaacctcaagtgatccgcccgcctcaccctccagaagtgctaggattacaggcctgagccaccgcacccggccctgctatttttttctatgtagttgtttcagtaataccatatgagcagagaatactattgattcattcaattgaagaaatctgaataaattagtaaatataataattcttcaaaagaggaccactttatgtttccaatagattatataattgaattcaggtaacgtagagttattcagacttttttttttaaataacggaattttgcaaaaaaacaggttgcatcgttttctacagagaatattcagatgtgtagaggcaaagagactttcttgaaaggtttttatttcctgttaaatttgtgaggaaaactcctgagaaaggaaagatttacttttgaaggaagaaagaacataacccaaagaaatttgaaagattcaaaaagagttttccaaatgtattctcatttctttctcatatttgttcaaggtcttagtttccaagcattcaaaattaaatcttgtttgcctaactcttctaacatctattaccaacctaacatattacaattctgtttaaaatgtgcagttctaaatgttcttgcgtgcctcactgtggaaagtgtggtgcatggaactgcagcgtcagtatcacccagaagtataattacaagaagtgcagagtccttggccttaccctgtacctacagaagtccgcttttgggcaagatccccagatgattcatatgtgtattaatattgaaaaaattggaactagtaagtcctagaggaggtaaattttaagggctttttaagggaacttccaagtgtgaagacatttctggagatgacaagtcaacatagcatgggatcttggctagacaaggaaagcgagagaacattaaataaacacaagcaacgacaaaccacctatgttataattttatcctggaccatccaaagtttactcttggtcacgctgtctaacccagcctgcctcctggcacttgctagttagaaccagggaagagaaaactaatatgtgttaggtattataatttccttatgtcacataaattttggtgccatagctagcaatgtttcttaatatcacatcagtacaatgtgtccattgccttctgtaacatttttaatgctatcaataatacattgacattataaaaataaatgaatgtggtcaaaaaaaaaaaaaaaaaa

SEQ ID NO: 26 - DNA, 7911 bp, Mus musculus
  misc_feature (1)..(7911): Mus musculus hyperpolarization-activated, cyclic nucleotide-gated K+ 1 (Hcn1)
aatgtcacgctgggcagaagtggcacaggcgagcggcggggcgcgcgcgggtagaggcggtctgcggccagcagggggcagccgaggggcggcgctggcgcgcgggtgccgagcggaggagcctcgccccgcgcccgcagcctcagcctcagcctcagtaccaggcgcggcggagcccggagcgcggcagagccggcagcgcagccactgagggcagcggcggcggcgggagccaggcgcgcagcggagcagcgggcgggcgggaagcagtagccgccgccgccgctgccgccgcgacgggcagccggctccggcccctgctccttggcttcgagcccccggcgagtctggagcccgcgccgtcgccggtcgcgtcctccgggcatggaaggcggcggcaaacccaactccgcgtccaacagccgcgacgatggcaacagcgtcttcccctccaaggcgcccgcgacggggccggtggcggccgacaagcgcctggggaccccgccggggggcggcgcggccgggaaggaacatggcaactccgtgtgcttcaaggtggacggcggcggaggagaggagccggcgggcagcttcgaggatgccgaggggccccggcggcagtatggtttcatgcagaggcagttcacctccatgctgcagcctggggtcaacaaattctccctccgcatgtttgggagccagaaggcggtggagaaggagcaggaaagggttaaaactgcaggcttctggattatccatccgtacagtgacttcaggttttattgggatttaatcatgcttataatgatggttggaaatttggtcatcataccagttggaatcacgttcttcacagagcagacgacaacaccgtggattattttcaacgtggcatccgatactgttttcctgttggacttaatcatgaattttaggactgggactgtcaatgaagacagctcggaaatcatcctggaccctaaagtgatcaagatgaattatttaaaaagctggtttgtggtggacttcatctcatcgatcccggtggattatatctttctcattgtagagaaagggatggactcagaagtttacaagacagccagagcacttcgtatcgtgaggtttacaaaaattctcagtctcttgcggttattacgcctttcaaggttaatcagatacatacaccagtgggaagagatattccacatgacctatgacctcgccagtgctgtggtgaggatcttcaacctcattggcatgatgctgcttctgtgccactgggatggctgtcttcagttcctggttcccctgctgcaggacttcccaccagattgctgggtttctctgaatgaaatggttaatgattcctggggaaaacaatattcctacgcactcttcaaagctatgagtcacatgctgtgcattggttatggcgcccaagcccctgtcagcatgtctgacctctggattaccatgctgagcatgattgtgggcgccacctgctacgcaatgtttgttggccatgccacagctttgatccagtctttggactcttcaaggaggcagtatcaagagaagtataagcaagtagagcaatacatgtcattccacaagttaccagctgacatgcgccagaagatacatgattactatgagcaccgataccaaggcaagatcttcgatgaagaaaatattctcagtgagcttaatgatcctctgagagaggaaatagtcaacttcaactgccggaaactggtggcaactatgcctctttttgctaacgccgatcccaatttcgtgacggccatgctgagcaagctgagatttgaggtgttccagcccggagactatatcattcgagaaggagctgtggggaagaaaatgtatttcatccagcacggtgttgctggcgttatcaccaagtccagtaaagaaatgaagctgacagatggctcttacttcggagagatatgcctgctgaccaagggccggcgcactgccagtgtccgagctgatacctactgtcgtctttactccctttcggtggacaatttcaatgaggtcttggaggaatatccaatgatgagaagagcctttgagacagttgctattgaccgactcgatcggataggcaagaaaaactctattctcctgcagaagttccagaaggatctaaacactggtgttttcaacaaccaggagaacgagatcctgaagcagatcgtgaagcatgaccgagagatggtacaagctatccctccaatcaactatcctcaaatgacagccctcaactgcacatcttcaaccaccaccccaacctcccgcatgaggacccaatctccgccagtctacaccgcaaccagcctgtctcacagcaatctgcactcacccagtcccagcacacagacgccccaaccctcagccatcctttcaccctgctcctataccacagcagtctgcagtcctcctatacagagccccctggccacacgaactttccattatgcctctcccactgcgtcccagctgtcactcatgcagcagcctcagcagcaactaccgcagtcccaggtacagcagactcagactcagactcagcagcagcagcagcaacagcagcagcagcagcagcagcaacagcaacaacagcagcagcagcagcagcagcagcagcagcagcagcagcagcagcagcagcagcagcagccacagacacctggtagctccacaccgaaaaatgaagtgcacaagagcacacaagcccttcataacaccaacctgaccaaagaagtcaggcccctttccgcctcgcagccttctctgccccatgaggtctccactttgatctccagacctcatcccactgtgggcgaatccctggcctctatccctcaacccgtggcagcagtccacagcactggccttcaggcagggagcaggagcacagtgccacaacgtgtcaccttgttccgacagatgtcctcgggagccatcccccccaaccgaggagtgcctccagcaccccctccaccagcagctgtgcagagagagtctccctcagtcctaaatacagacccagatgcagaaaaaccccgttttgcttcgaatttatgattcttgatgattgtcaaagcagaaaagaagtactctaataaacagaatattctcagatattattttattctatctcacgatagatccctatagcctactctgaaaaaaaaatattttagaagctgtggcctacatgcaaatgtaaaaacatatatacatatataattaaatatatatatatatatatatatctaaatgcccaagagaagttcaaaagacttgtataacattcagtgttacatgtcttcctctctttaaaaccattaaaggatttaacacattgttgtaaggtcattgatttctaaccttttacttgattcctttgatatatgtgtttctcccttttatgaagagttcttggagtcaatggaaacaaaactcttgattttaaaaaaggcaactccaattagatccagcatagcaccaatcaaagctttctttcattagctgtgcctctgcgtctaggttgttaattatgcgggattcaataaacaaatcccagtttatagctctcaattgtattttggtgctttaaattttgagttaggtgaaggaacacgctccatgctcagccaccgtaggagactaacattgccattaaggcttcctctaacctcaaacatgttcattgattttgtgagaaaagtgaggagatatttgtcttcatgtgtcactggacttttaccaagacgcagtcaatgttagctgtaaataactttcccagcccgaattaaagtaactattctgtgttgtagaaaggtaaaagtcagtgtttaagaatttagttttactgcttcacttcaaaagttagagttttaaatttcacaaaacactctaattgtgacagctgttcaaatgtaatgcaatggcttgagacctacaatatcatttttaacctgcgatactttaatgcaaaaattgtatgcttgaacctacaaattgcttgtatgacaccaaaaatcattacttttattccttcttgacataatcaagcacctgaacctggtcctggcatgctttcgggggcaaaaaaaaataaaataaaataacttaaaaaaaaaagtagattcaaatcaatcattgtaagaaaaggctgacagtatttctcacttctagatgagtataactattcattaattgcctctcttaaaattcttaaaaggctgacttggatctctgacttaaatctagaagtgaggttttcactttcatttgatattgttgctgttcttattgtttaagcaatcatttgcaaaccatagcttgttttggagttaaaggtgttatgtgtcttcatgttagttggtagtttattgtgatgttaatgtgaagaaataaatactatatatcaccacatctgtttggaaatacacattttgccctttgtatattgtaactaagcagttagtaaatcttaaggcatgtggtaacctacatagtccttagatagacaggaggtttgttctttcaaatgtgctgaaacttatatggactccgtgaggggaagcaggggggaattgagctgtctgaaagcattgtctgtttgttcacttgtttccttaattctttctgtgagcatcatggagcctaatctagggaagtcccttagcgatcttgcttcatgtgtgtttgctgcacctcacacaagtggtgggtgagtagaacacagtagcaggctctctagtgcctgacttctgtacgtaactcaagagagtgagctgatatggaggtctttgacattacaggcacaaatgatctatttaagaacataaagaccatacattctattgggacagggcctccaggtatccttgaggaccaagaacaaaagtagaagcaagaggtttaaagtggatctacattgccttttatgaaaccaaaatgagtcgttgcacttaattgctaacggatcctaccttgtgtgaaacaatcagcaatgctaggtaacgtgatgtcattcctgtaatgctagtgaccaatggtatcctgttaggaatgatgtaagagagagatcctgcagcctacgtaaaatctatgggaaagttgcaagacctatcacatatcagtacaaaactcctttattttattaaatttaaaaattagcttcttgttaagatcatgtactagtcctttaaattattaaaggtctacctttggcaggattaatgcatttcgtagtaaaatatttttatatatcaattttttgagcactattggagaacatccgcaaaattgaattgcatggttattcagtgcaaccaataaaatttttattcactttgtactcgaatggacaaagaacactaaggtttatatgttaaagtaacaaaaccatacagggtatattaaatacacatatgtatgaatgtgtgtgtttgtaaaggtacagtgctaggagaacatgaccttttttctctgtacatagttaagctcaagtacggagttttaatctgtacgggaagagctgtattggggtcttttactgctgagaaagtagaacaataacttatactttgcactgtatgaccagggaagagttagagtatttcttttgtctttgctcaaagccatacatatataaatatatattaagattattaataaattcaaaagaaaataaaccaaattatgaatattttattttgttgttgaaatctaatcaaatgtttaactcattgaggagtacagatttgcttgttgttctctctttgtataatctccaaaaagatttgtttttgaaaatcaccatacaattgttatagaaattctacagatatagaatgaatgtaatttaaatagaacatagatgtctcagtcacctgtttgttttcctttttttcatatggtaacatttggttatcttattctttaccccatgcctctttagtatgtggtgtttctccagttttaaaatgcagccatgtctttgttattggatatggggatggtttttaattttcgtacatgacctgttttctctgggcttttcaaaggcctcagatgagatttgtcacaatgaggataaatagcaaagcagtcctaaacacaaggaggataaagtgtgattcttcaattggttgtcctcctcaaacacacagcctgactccagatctccgtgacctctccataagtgaatggttaccaagtggatgccattctacttttaaaagatgcttactctatcatttacagacttaaatgaacatgttattttaaggacactgcaaagaaagcagtcgctagtctaataattcctacacatacgaacaatttaaacaatatgatgagtttcaacttttagatacattatactaaaatcagaattagttatgattcaacatataagtcaccttgactccttgacaagattattgtgtaaaatacctccatatatttttatgacttgttttttatttggtaagtttactataattgctatttgggatcttactttccttatttttataatattaaactagatgactaaaatttttctcatttttatcttctccatccttgtagagcttgtatcatgccatgcaagtgtaggatatgttgttgttgctgctacagctgctgctattgttgttaatattcactaaccctaacaaaaattttctatatgataggtatatctacctaaatatccataagtctgtgccataagaaaaaaactatactcaagtattaaactacactgaattctgaatagtcaaaaattgtcacgaaacatgcagttgaatatgtggaccaaattatgtgggaatttttttaactttattcaaaatagttcctgaaatattttcaaaatcatgttggaaattttctttcgaggattttttttttttatcaatttaagggtgatttgtagttgctgtgaccacttgaaaatagtgactttctaaagcagttttatttcattttaataaataacacgtttttattcttttctcatatatttattctgatacaattttcccaaccccctcttcccccagcctcttccctcccccatcaccactctcttccatatccccacccctctcctggggaaaaggatcgacatttttattgcatgtattgatatcttcacaaacaggtcattttggccttgatcacatttatagctttcaggtaaaaacaaaaaacaaaaaacaaaaccataagcttctccatatagcaaaaatattcaggttgaataataaatctgtcaccacaataaatcctctcattctagttcaaaggatacgtctgtgatatttgctctgttgcttaatgctttgtgcagctctctcgatgaaatgttattactatcttttcttcatatggaaatagtctctgtaacacaaggtgatgaggaatgaagataaatttccaaaaaaaaaaaaaatctggaatgagataaatggtatatttgttccatgtacaaatgttgatgtctcaaaacaagacaagtgatgttttataaaatcaaaataagtgtgatggccataacccaagtattccattttaaattaagacgggacccaaccccgcaatcacgtgaacatgcctctcttatctcaccatgcctctcttatctcaccataatcctaccccagtttccctaaccaagggcggtgcatagcgacaagtgaagactggaaagactttgttgaaagaagccttgccaagacttggttttggcctatcatgagacttgttaaagatgataataaagggaaatgtatggaagtctattaatttatgtctgagatacttgatgtagaaagaataaaatttgaagaaagcacc

SEQ ID NO: 27 - DNA, 7245 bp, Homo sapiens
  misc_feature (1)..(7245): Homo sapiens hyperpolarization activated cyclic nucleotide-gated potassium channel 4 (HCN4)
caaaaatgccagggaaaggcgagcccagagcttggtgatggagaaattgggaagccaccccccacccttcaatcttaggatggggaattcgcaactgaagccggagcttcagacttggggcgcactcccagcttagcccaggaaagagatttaagggcgcagcagtgtggatacctctcaccccggccccgaaggtctagcgagggtctaacctgggccccttgccaggcccgccccccgcccctttccagcccccggcccgtgcgccgctgcccctttaagaagcccaggtaggcaggcccggctgctggagccgctcctatggcaacccgcgagctgcggcggcttcatgaatattccggggcgcgggagcccgagcgctgccggagggcgcttcgggggaggcggccgctgatgtaagcccggcgggtcgctgggctccgctcggttgcggcgggagccccgggacgggccggacgggccggggcagaggaggcgaggcgagctcgcgggtggccagccacaaagcccgggcggcgagacagacggacagccagccctcccgcgggacgcacgcccgggacccgcgcgggccgtgcgctctgcactccggagcggttccctgagcgccgcggccgcagagcctctccggccggcgcccattgttccccgcgggggcggggcgcctggagccgggcggcgcgccgcgcccctgaacgccagagggagggagggaggcaagaagggagcgcggggtccccgcgcccagccgggcccgggaggaggtgtagcgcggcgagcccggggactcggagcgggactaggatcctccccgcggcgcgcagcctgcccaagcatgggcgcctgaggctgcccccacgccggcggcaaaggacgcgtccccacgggcggactgaccggcgggcggacctggagcccgtccgcggcgccgcgctcctgcccccggcccggtccgaccccggcccctggcgccatggacaagctgccgccgtccatgcgcaagcggctctacagcctcccgcagcaggtgggggccaaggcgtggatcatggacgaggaagaggacgccgaggaggagggggccgggggccgccaagaccccagccgcaggagcatccggctgcggccactgccctcgccctccccctcggcggccgcgggtggcacggagtcccggagctcggccctcggggcagcggacagcgaagggccggcccgcggcgcgggcaagtccagcacgaacggcgactgcaggcgcttccgcgggagcctggcctcgctgggcagccggggcggcggcagcggcggcacggggagcggcagcagtcacggacacctgcatgactccgcggaggagcggcggctcatcgccgagggcgacgcgtcccccggcgaggacaggacgcccccaggcctggcggccgagcccgagcgccccggcgcctcggcgcagcccgcagcctcgccgccgccgccccagcagccaccgcagccggcctccgcctcctgcgagcagccctcggtggacaccgctatcaaagtggagggaggcgcggctgccggcgaccagatcctcccggaggccgaggtgcgcctgggccaggccggcttcatgcagcgccagttcggggccatgctccaacccggggtcaacaaattctccctaaggatgttcggcagccagaaagccgtggagcgcgaacaggagagggtcaagtcggccggattttggattatccacccctacagtgacttcagattttactgggacctgaccatgctgctgctgatggtgggaaacctgattatcattcctgtgggcatcaccttcttcaaggatgagaacaccacaccctggattgtcttcaatgtggtgtcagacacattcttcctcatcgacttggtcctcaacttccgcacagggatcgtggtggaggacaacacagagatcatcctggacccgcagcggattaaaatgaagtacctgaaaagctggttcatggtagatttcatttcctccatccccgtggactacatcttcctcattgtggagacacgcatcgactcggaggtctacaagactgcccgggccctgcgcattgtccgcttcacgaagatcctcagcctcttacgcctgttacgcctctcccgcctcattcgatatattcaccagtgggaagagatcttccacatgacctacgacctggccagcgccgtggtgcgcatcgtgaacctcatcggcatgatgctcctgctctgccactgggacggctgcctgcagttcctggtacccatgctacaggacttccctgacgactgctgggtgtccatcaacaacatggtgaacaactcctgggggaagcagtactcctacgcgctcttcaaggccatgagccacatgctgtgcatcggctacgggcggcaggcgcccgtgggcatgtccgacgtctggctcaccatgctcagcatgatcgtgggtgccacctgctacgccatgttcattggccacgccactgccctcatccagtccctggactcctcccggcgccagtaccaggaaaagtacaagcaggtggagcagtacatgtcctttcacaagctcccgcccgacacccggcagcgcatccacgactactacgagcaccgctaccagggcaagatgttcgacgaggagagcatcctgggcgagctaagcgagcccctgcgggaggagatcatcaactttaactgtcggaagctggtggcctccatgccactgtttgccaatgcggaccccaacttcgtgacgtccatgctgaccaagctgcgtttcgaggtcttccagcctggggactacatcatccgggaaggcaccattggcaagaagatgtacttcatccagcatggcgtggtcagcgtgctcaccaagggcaacaaggagaccaagctggccgacggctcctactttggagagatctgcctgctgacccggggccggcgcacagccagcgtgagggccgacacctactgccgcctctactcgctgagcgtggacaacttcaatgaggtgctggaggagtaccccatgatgcgaagggccttcgagaccgtggcgctggaccgcctggaccgcattggcaagaagaactccatcctcctccacaaagtccagcacgacctcaactccggcgtcttcaactaccaggagaatgagatcatccagcagattgtgcagcatgaccgggagatggcccactgcgcgcaccgcgtccaggctgctgcctctgccaccccaacccccacgcccgtcatctggaccccgctgatccaggcaccactgcaggctgccgctgccaccacttctgtggccatagccctcacccaccaccctcgcctgcctgctgccatcttccgccctcccccaggatctgggctgggcaacctcggtgccgggcagacgccaaggcacctgaaacggctgcagtccctgatcccttctgcgctgggctccgcctcgcccgccagcagcccgtcccaggtggacacaccgtcttcatcctccttccacatccaacagctggctggattctctgcccccgctggactgagcccactcctgccctcatccagctcctccccaccccccggggcctgtggctccccctcggctcccacaccatcagctggcgtagccgccaccaccatagccgggtttggccacttccacaaggcgctgggtggctccctgtcctcctccgactctcccctgctcaccccgctgcagccaggcgcccgctccccgcaggctgcccagccatctcccgcgccacccggggcccggggaggcctgggactcccggagcacttcctgccacccccaccctcatccagatccccgtcatctagccccgggcagctgggccagcctcccggggagttgtccctaggtctggccactggcccactgagcacgccagagacacccccacggcagcctgagccgccgtcccttgtggcaggggcctctgggggggcttcccctgtaggctttactccccgaggaggtctcagcccccctggccacagcccaggccccccaagaaccttcccgagtgccccgccccgggcctctggctcccacggatccttgctcctgccacctgcatccagccccccaccaccccaggtcccccagcgccggggcacacccccgctcacccccggccgcctcacccaggacctcaagctcatctccgcgtctcagccagccctgcctcaggacggggcgcagactctccgcagagcctccccgcactcctcaggggagtccatggctgccttcccgctcttccccagggctgggggtggcagcgggggcagtgggagcagcgggggcctcggtccccctgggaggccctatggtgccatccccggccagcacgtcactctgcctcggaagacatcctcaggttctttgccaccccctctgtctttgtttggggcaagagccacctcttctggggggccccctctgactgctggaccccagagggaacctggggccaggcctgagccagtgcgctccaaactgccatccaatctatgagctgggcccttccttccctcttctttcttcttttctctcccttccttcttccttcaggtttaactgtgattaggagatataccaataacagtaataattatttaaaaaaccacacacaccagaaaaacaaaagacagcagaaaataaccaggtattcttagagctatagatttttggtcacttgcttttatagactattttaatactcagcactagagggagggagggggagggaggagggagcaggcaggtcccaaatgcaaaagccagagaaaggcagatggggtctccggggctgggcaggggtgggagtggccagtgttggcggttcttagagcagatgtgtcattgtgttcatttagagaaacagctgccatcagcccgttagctgtaacttggagctccactctgcccccagaaaggggctgccctggggtgtgccctggggagcctcagaagcctgcgaccttgggagaaaagggccagggccctgagggcctagcattttttctactgtaaacgtagcaagatctgtatatgaatatgtatatgtatatgtatgtaagatgtgtatatgtatagctatgtagcgctctgtagagccatgtagatagccactcacatgtgcgcacacgtgtgcggtctagtttaatcccatgttgacaggatgcccaggtcaccttacacccagcaacccgccttggcccacaggctgtgcactgcatggtctagggacgttctctctccagtcctcagggaagaggaccccaggacttcgcagcaggccccctctctccccatctctggtctcaaagccagtcccagcctgacctctcaccacacggaagtggaagactcccctttcctagggcctcaagcacacaccgccacctctggggccgtcagtttgcccatctgtacagtgggaggtgagcggaacttctgtttattgagtctgctctgtgccaagcactggtttcgcactttacacacattaactccttcagtttcacaaagaccatggggtgggtactttgattctccccatttagcagaggaagaaacagttttgggtaatttttccagaatcatgtaactaggagtggcagagtggggactgatttgaggttcgagtccacgcctccttgaggcccaagtctgtgttccttccatcagaaaactgtgttgaggggggctgaggtagatggtccccaagcatggtacagaaggaagacaccagattttggcagcagtcaggcctgggtttgaatcccagccctgccacttcttagctgtatgatcttgggcaagttatctgacctttctgtcacctcatttgtaaaatgggaataattatggtactgcctcacaaggacctatgaggaccagatgagaaaaatctatatgtgaaatgcccagcccagcgcctggcacataccatggtaggtgctcaataaaaaatcacatttcttctgcccctcatatgcccagcctattgctccagcaaactatgtgagagcccagggagctttggctgagggctccaagacttaaaatctcaggactcaggaggtggctgggcctccctaagggcccaaggaaggtgtgtggccagaggtgggtgggagccaggccttgagaagtgggaagacttcaacagggagagagggagggaatggtgggtgggatggagtgtatggtggggagattcctgaggtggatgtggagtggtggatcagggctttgggaggggatccccaggctgaggggtcagagggacggccttgggtgatagggtaagggattgtctgggcttagtcctggcaactaggagccataagcaggttccagattgcgggaacgagaaagcagctcagatgcctttggaggcaccatcctccctcctcccagatgggatcttgccagagccaaggtcaggggtctgcccctgcctatagggccagagcaggtatggctgcaatccccaagtaatgagaagggctggtcccacattatccatccagaaccttccatgctccaagccagaatgttggcaagatcgggttttgccttgagctatcctgggatgtgagacaaaccgatttctccatagatgggctgcagggagtgggaggcagtactccaggagagaagtgggtgaaggttcctgggatcttaggtaaagactagacgccgcctagtactggtctctactgtgctggctcaggagttctgagaactggaaggacttagcctcaacctgagttctgcacacaccccttccccttaaggaaggcagctctgagaggcagcaggacttgatccaaacccacagtcttgtcctggaggcagcaggggtgaaggtggagggtccagggccatgaggagcccccttgccatcagagcctggcctaaccaccctcttctctacttacacacacatgcattttataatagctctgacccaacctggccactctgcagagactgggacagacaggtgcaggcaatgggccctcccacacccagtcacctacaaggaattttcaaatccacttttaaaacagaaaccggtaaatgcgccgtattgtatattttatttaaataaaaaaaattccagcaaaaaaaaaaaaaaaaaa

SEQ ID NO: 28 - DNA, 3798 bp, Mus musculus
  misc_feature (1)..(3798): Mus musculus hyperpolarization-activated, cyclic nucleotide-gated K+ 4 (Hcn4)
atggacaagctgccgccgtccatgcgcaagcggctctacagccttccgcagcaggtgggggccaaggcgtggatcatggacgaggaagaggatggtgaggaagaaggggccgggggccgccaggaccccagccgaaggagcatccggctgcggccgctgccctcgccctctccctcggtggctgcgggctgctcggagtcccggggtgcggccctcggggcgacagagagcgaggggccgggccgcagcgccggcaagtccagcaccaacggtgactgcaggcgcttccgcgggagtctggcctcgctgggcagccggggcggcggcagtggtggagcagggggcggcagcagtctcgggcacctgcatgactccgcggaggaacggcggctcatcgccgctgagggcgatgcgtcccccggcgaggacaggacgcccccgggcctggcgaccgaacccgagcgcccggccaccgcggcacaacccgcagcctcgccgccgccccagcagccgccgcagccggcctctgcctcctgcgagcagccctcggcggacaccgctatcaaagtggagggaggcgcggccgccagcgaccagatcctccccgaggccgaggtgcgcctgggccagagcggcttcatgcagcgccagttcggtgccatgctgcaacctggggtcaacaaattctccctaaggatgttcggcagccagaaagcggtggagcgcgagcaggagagggttaagtcagcagggttttggattatccacccctacagtgacttcagattttactgggacctgacgatgctgttgctgatggtggggaatctgatcatcatacccgtgggcatcaccttcttcaaggatgagaacaccacaccctggatcgtcttcaatgtggtgtcagacacattcttcctcattgacttggtcctcaacttccgcacggggatcgtggtggaggacaacacagaaatcatccttgacccgcagaggatcaagatgaagtacctgaaaagctggtttgtggtagatttcatctcctccatccctgtcgactacatcttccttatagtggagactcgcattgactcggaggtctacaaaaccgctagggctctgcgcattgtccgtttcactaagatcctcagcctcctgcgcctcttgaggctttcccgcctcattcgatacattcatcagtgggaagagatcttccacatgacctatgacctggccagcgccgtggtacgcatcgtgaacctcattggcatgatgcttctgctgtgtcactgggatggctgcctgcagttcctagtgcccatgctgcaggacttcccccatgactgctgggtgtccatcaatggcatggtgaataactcctgggggaagcagtattcctacgccctcttcaaggccatgagccacatgctgtgcattgggtatggacggcaggcacccgtaggcatgtctgacgtctggctcaccatgctcagcatgatcgtgggggccacctgctatgccatgttcatcggccacgccactgccctcatccagtcgctagactcctcccggcgccagtaccaggagaagtataaacaggtggagcagtacatgtccttccacaagctcccgcctgacacccgacagcgcatccatgactactatgaacaccgctaccaaggcaagatgtttgatgaggaaagcatcctgggtgagctgagtgagccacttcgagaggagatcatcaactttaactgccgaaagctggtggcatccatgccactgtttgccaacgcagatcccaactttgtgacatccatgctgaccaagttgcgtttcgaggtcttccagcctggggattacatcatccgcgaaggcaccatcggcaagaagatgtactttatccagcacggcgtggtcagcgtgctcactaagggcaacaaagagaccaagctggctgatggctcctattttggagagatctgcttgctgacccggggtcggcgcacagccagcgtcagagcggatacttattgccgcctctactcactgagcgtggacaacttcaatgaggtgctggaggagtatcccatgatgcggagggccttcgagacggttgcgctggaccgcctggaccgcataggcaagaagaactccatcctcctccacaaggtgcagcacgacctcaactcaggcgtcttcaactaccaagagaacgagatcatccagcagatcgtgcggcatgaccgtgagatggcccactgtgctcaccgcgtccaggctgccgcctcagccaccccaacccccacgcctgttatatggaccccgctgatccaggcgccactgcaggctgctgctgctactacttcggtggccatagccctcacacaccacccccgcctgcccgccgccatcttccggccccctcccggacctgggctgggcaaccttggggctggacagacaccgaggcacccaaggaggctgcagtccttgatcccttcagctctgggctctgcttcacccgccagcagcccctcacaggtggacacaccgtcttcatcctccttccacatccaacagctggctggattctctgcacctcctggattgagccctctcctgccctcctctagctcttccccacctccaggagcctgcggttccccaccagcccccacaccctccacctccactgccgccgccgcctccaccactgggttcggccactttcacaaggcgctgggtggctccctgtcatcctctgactccccgctgctcaccccactgcaaccaggcgctcgctctccacaggctgcccagccaccacccccactgcctggggcccgaggaggtctgggactcctggagcacttcttgccgcccccaccctcctccaggtcaccatcatccagccctgggcagctgggccagcctcctggagagttgtccctaggtctggcagctggtccatcaagtacaccagagacacccccacggcctgagcgaccatccttcatggcaggggcctctggaggggcttctcctgtagcctttaccccccgaggaggcctcagtcctccgggccacagcccggggcccccaagaactttcccgagtgccccaccccgggcctctggctcccatggttccctgctcctgccacctgcatccagccctccacctccccaggtcccacagcgcaggggcacaccacccctcacccctggccgcctcacacaggacctgaagctcatctcagcctctcagccagccctcccccaggatggggcacagactctccgcagggcctcgcctcactcctcaggggagtcggtggctgccttctcactctaccccagagctgggggtggcagtgggagtagtgggggccttgggcctcctggaaggccatatggtgccatcccaggccaacatgtcactttgcctcggaagacatcctcaggttctttgccacccccactttctttgtttggggcaagagccgcctcttctggagggccccctctgactactgctgcaccccagagggaacctggcgctaggtctgagccagtacgctccaaactgccgtctaatttatgagctgggccctccctccctcttctttttcttgctcctttcttccttcaggtttaactgtgattaggagatataccaacaacaataataaccataaaaaaacataccccagaaaaacaaaaagacagcagaaaataaccaggtattcttagagctatagatttttggtcacttgcttttatagactattttaat